(19) United States
(12) Patent Application Publication
(10) Pub. No.: US 2024/0293569 A1
(43) Pub. Date: Sep. 5, 2024

(54) LINKER FOR ANTIBODY-DRUG CONJUGATE

(71) Applicant: PINOTBIO, INC., Suwon-si (KR)

(72) Inventors: Doo Young JUNG, Suwon (KR); Hyun Yong Cho, Suwon (KR); Gangadhar Rao Mathi, Suwon (KR)

(73) Assignee: PINOTBIO, INC., Suwon-si (KR)

(21) Appl. No.: 18/437,879

(22) Filed: Feb. 9, 2024

Related U.S. Application Data
(60) Provisional application No. 63/529,828, filed on Jul. 31, 2023.

(30) Foreign Application Priority Data
Feb. 10, 2023 (KR) 10-2023-0017886
Feb. 24, 2023 (KR) 10-2023-0024745

(51) Int. Cl. A61K 47/68

(52) U.S. Cl. CPC A61K 47/6889; A61K 47/68037

(57) ABSTRACT

Provided herein are a linker compound, a linker-drug conjugate where the linker compound is conjugated to a drug, an antibody-drug conjugate where a drug is conjugated to an antibody or an antigen binding fragment thereof via the linker compound, and a method for treating cancer by administering the antibody-drug conjugate to a subject in need thereof.

### CROSS REFERENCE TO RELATED APPLICATION

The present application claims priority to U.S. Patent Application No. 63/529,828 filed Jul. 31, 2023, Korean Patent Application No. 10-2023-0017886 filed Feb. 10, 2023, and Korean Patent Application No. 10-2023-0024745 filed Feb. 24, 2023, the entire disclosures of which are incorporated herein by reference.

### FIELD

Provided herein are a linker compound, a linker-drug conjugate where the linker compound is conjugated to a drug, an antibody-drug conjugate where a drug is conjugated to an antibody or an antigen binding fragment thereof via the linker compound, and a method for treating cancer by administering the antibody-drug conjugate to a subject in need thereof.

### BACKGROUND

Several antibody-drug conjugates (ADC) that are either approved or in various clinical stages, for antitumor applications encounter safety challenges characterized by adverse side effects and inherent toxicity. Despite the commendable antitumor efficacy exhibited by these ADC, their clinical utility is hindered by safety considerations. This obstacle underscores the imperative of advancing therapeutic modalities of ADCs characterized by both exceptional efficacy and an improved safety profile.

80% of the approved ADC use cleavable linkers for payload delivery to tumor. A limitation associated with cleavable linkers pertains to the potential shedding of payload during the circulation of Antibody-Drug Conjugates (ADCs) before reaching the tumor target. Premature release of the payload systemically can diminish the potency of the remaining ADC in circulation, potentially resulting in non-specific uptake and off-target toxicities that may be dose-limiting. These challenges significantly influence the development of ADC drugs. This not only complicates the assessment of antitumor efficacy in preclinical studies involving mice, but also restricts ADC dosing in patients, thereby hindering the realization of its full therapeutic potential.

### SUMMARY

To overcome the above limitations, the present disclosure provides a novel cleavable linker system distinguished by excellent therapeutic efficacy and an exemplary safety profile.

(1) In particular, the present disclosure provides a compound of Formula (1):

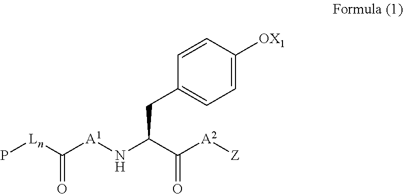

wherein
P is -(maleimide-N)—, -(dibromomaleimide-N)—, -(bromoacetamide-N)—, —(Y—CH₂)—, or -(dibenzocyclooctyne-N—C(═O))—, Y being Br, Cl or I, and the -(dibenzocyclooctyne-N—C(═O))— having the structure of

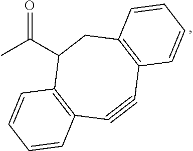 [SMILES: CC(=O)C1Cc2ccccc2C#Cc2ccccc21]

Lₙ is optionally included, and is cycloalkyl, alkyl, or —(OCH₂CH₂)—, —(CH₂—C(═O)—NH—CH₂—CH₂)— if included, and n is an integer of 1 to 6;
A¹ is a peptide residue containing 0-3 amino acids;
A² is a peptide residue containing 0-3 amino acids;
Z is optionally included, and is a self-immolative spacer, —(NHCH₂)—, or p-aminocarbamate if included; and
X₁ is H, monosaccharide, disaccharide, oligosaccharide, polyethylene glycol, sulfate, phosphate, or pyrophosphate.

(2) In addition, the present disclosure provides a linker-drug conjugate comprising:
the compound according to the embodiment mentioned above (1); and
a drug which is conjugated to the compound via Z of Formula (1).

(3) The present disclosure also provides the linker-drug conjugate according to the embodiment mentioned above (2), wherein the drug is conjugated to the compound in the form of —(NH-drug)- or —(O-drug)-.

(4) The present disclosure also provides the linker-drug conjugate according to the embodiment mentioned above (2), wherein the drug is a topoisomerase I inhibitor.

(5) The present disclosure provides an antibody-drug conjugate comprising an antibody or an antigen binding fragment thereof, a drug, and a linker,
wherein the linker is the compound according to the embodiment mentioned above (1),
wherein the antibody or the antigen binding fragment thereof is conjugated to the compound via P of Formula (1), and
wherein the drug is conjugated to the compound via Z of Formula (1).

(6) The present disclosure provides a method of treating cancer, the method comprising administering an effective amount of the antibody-drug conjugate of the embodiment mentioned above (5) to a subject in need thereof.

(7) The present disclosure provides the method of the embodiment mentioned above (6), wherein the cancer comprises one or more of breast cancer, liver cancer, skin cancer, ovarian cancer, cervical cancer, prostate cancer, testicular cancer, brain cancer, clear cell renal cell carcinoma, glioma, melanoma, lung cancer, non-small cell lung cancer (NSCLC), small cell lung cancer, pancreatic cancer, gastric cancer, acute myeloid leukemia (AML), Hodgkin's lymphoma, non-Hodgkin's lymphoma (NHL), colorectal cancer, colon cancer, renal cancer, esophageal cancer, leukaemia, hepatocellular carcinoma, bone cancer, bladder cancer, sarcomas, kidney cancer, head and neck cancer, hypopharyngeal squamous cell carcinoma, glioblastoma, neuroblastoma, endometrial cancer, and urothelial cell carcinoma.

(8) The present disclosure also provides a compound of Formula (2):

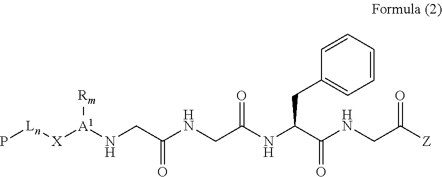

wherein
P is -(maleimide-N)—, -(dibromomaleimide-N)—, -(bromoacetamide-N)—, or -(dibenzocyclooctyne-N—C(═O))—;
Lₙ is optionally included, and is cycloalkyl, alkyl or a direct bond if included, and n is an integer of 1 to 6;
X is —(C═O)—, a direct bond, or a direct bond to a side chain of A¹;
A¹ is an amino acid;
Rₘ is a hydrophilic appendage attached to A¹ and m represents an integer of 1 to 10; and
Z is optionally included, and is a self-immolative spacer, —(NHCH₂)—, or p-aminocarbamate if included.

(9) The present disclosure provides the compound according to the embodiment mentioned above (8), wherein the amino acid is aspartic acid, glycine, glutamic acid or lysine.

(10) The present disclosure provides a linker-drug conjugate comprising:
the compound according to the embodiment mentioned above (8); and
a drug which is conjugated to the compound via Z of Formula (2).

(11) The present disclosure provides the linker-drug conjugate according to the embodiment mentioned above (10), wherein the drug is conjugated to the compound in the form of —(NH-drug)- or —(O-drug)-.

(12) The present disclosure provides the linker-drug conjugate according to the embodiment mentioned above (10), wherein the drug is a topoisomerase 1 inhibitor.

(13) The present disclosures provides an antibody-drug conjugate comprising an antibody or an antigen binding fragment thereof, a drug, and a linker,
wherein the linker is the compound according to the embodiment mentioned above (8),
wherein the antibody or the antigen binding fragment thereof is conjugated to the compound via P of Formula (2), and
wherein the drug is conjugated to the compound via Z of Formula (2).

(14) The present disclosure provides a method of treating cancer, the method comprising administering an effective amount of the antibody-drug conjugate of the embodiment mentioned above (13) to a subject in need thereof.

(15) The present disclosure provides the method of the embodiment mentioned above (14), wherein the cancer comprises one or more of breast cancer, liver cancer, skin cancer, ovarian cancer, cervical cancer, prostate cancer, testicular cancer, brain cancer, clear cell renal cell carcinoma, glioma, melanoma, lung cancer, non-small cell lung cancer (NSCLC), small cell lung cancer, pancreatic cancer, gastric cancer, acute myeloid leukemia (AML), Hodgkin's lymphoma, non-Hodgkin's lymphoma (NHL), colorectal cancer, colon cancer, renal cancer, esophageal cancer, leukaemia, hepatocellular carcinoma, bone cancer, bladder cancer, sarcomas, kidney cancer, head and neck cancer, hypopharyngeal squamous cell carcinoma, glioblastoma, neuroblastoma, endometrial cancer, and urothelial cell carcinoma.

(16) The present disclosure also provides a compound of Formula (3):

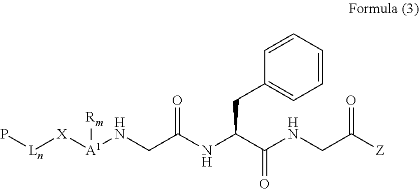

wherein
P is -(maleimide-N)—, -(dibromomaleimide-N)—, -(bromoacetamide-N)—, or -(dibenzocyclooctyne-N—C(═O))—;
Lₙ is optionally included, and is cycloalkyl, alkyl or a direct bond if included, and n is an integer of 1 to 6;
X is —(C═O)—, or a direct bond;
A¹ is an amino acid;
Rₘ is optionally included, and is a hydrophilic appendage attached to A¹ if included, and m represents an integer of 1 to 10; and
Z is optionally included, and is a self-immolative spacer, —(NHCH₂)—, or p-aminocarbamate if included.

(17) The present disclosure provides the compound according to the embodiment mentioned above (16), wherein the amino acid is aspartic acid, glycine, glutamic acid or lysine.

(18) The present disclosure provides a linker-drug conjugate comprising:
the compound according to the embodiment mentioned above (16); and
a drug which is conjugated to the compound via Z of Formula (2).

(19) The present disclosure provides the linker-drug conjugate according to the embodiment mentioned above (18), wherein the drug is conjugated to the compound in the form of —(NH-drug)- or —(O-drug)-.

(20) The present disclosure provides the linker-drug conjugate according to the embodiment mentioned above (18), wherein the drug is a topoisomerase 1 inhibitor.

(21) The present disclosure provides an antibody-drug conjugate comprising an antibody or an antigen binding fragment thereof, a drug, and a linker,
wherein the linker is the compound according to the embodiment mentioned above (16),
wherein the antibody or the antigen binding fragment thereof is conjugated to the compound via P of Formula (3), and
wherein the drug is conjugated to the compound via Z of Formula (3).

(22) The present disclosure provides a method of treating cancer, the method comprising administering an effective amount of the antibody-drug conjugate of the embodiment mentioned above (21) to a subject in need thereof.

(23) The present disclosure provides the method of the embodiment mentioned above (22), wherein the cancer comprises one or more of breast cancer, liver cancer, skin cancer, ovarian cancer, cervical cancer, prostate cancer, testicular cancer, brain cancer, clear cell renal cell carcinoma, glioma, melanoma, lung cancer, non-small cell lung cancer (NSCLC), small cell lung cancer, pancreatic cancer, gastric cancer, acute myeloid leukemia (AML), Hodgkin's lymphoma, non-Hodgkin's lymphoma (NHL), colorectal cancer, colon cancer, renal cancer, esophageal cancer, leukaemia, hepatocellular carcinoma, bone cancer, bladder cancer, sarcomas, kidney cancer, head and neck cancer, hypopharyngeal squamous cell carcinoma, glioblastoma, neuroblastoma, endometrial cancer, and urothelial cell carcinoma.

Additional embodiments are described herein.

### BRIEF DESCRIPTION OF THE DRAWINGS

FIG. 1 shows peptide cleavage specificity of cathepsin B and L. (J. Proteome Res. 2011, 10, 5363-5373).

FIG. 2 shows the SEC of Trastuzumab-LP2.

FIG. 3 shows HIC of Trastuzumab-LP2.

FIG. 4 shows in vitro data of Payload-001, Trastuzumab-LP1 and Trastuzumab-LP2

FIG. 5 shows SEC of Trastuzumab-LP3.

FIG. 6 shows HIC of Trastuzumab-LP3.

FIG. 7 shows HIC Comparison of Tra/Tra-LP1/Tra-LP3

FIG. 8 shows in vitro data of Trastuzumab-LP1 and Trastuzumab-LP3.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide” is a reference to one or more peptides and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

The term “antibody-drug conjugate,” as used herein, refers to the linkage of an antibody or an antigen binding fragment thereof with another antitumor compound, such as a chemotherapeutic agent, a toxin, an immunotherapeutic agent, an imaging probe, and the like. The linkage can be covalent bonds.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present disclosure may exist in a variety of forms including, for example, polyclonal antibodies, monoclonal antibodies as well as single chain antibodies and humanized antibodies.

The term “antigen binding fragment thereof,” as used herein, refers to a portion of a full length antibody, generally the antigen binding or variable region thereof. Examples of the antibody fragment include a Fab fragment, a Fab′ fragment, a Fab′-SH, a Fv fragment, a scFv fragment, a F(ab′)2 fragment, a VL fragment, a VH fragment, a ScFv-Fc fragment, a (ScFv)2-Fc fragment, diabodies, linear antibodies, fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules, and multispecific antibodies formed from antibody fragments.

The term “linker,” as used herein, refers to a moiety that connects two moieties of the antibody and the antitumor compound by covalent connections. In some instances, the term linker “linker” as used herein may refer to a moiety comprising a cleavable element, as well as further elements such as a connecting group, a group comprising one or more solubilizing groups, etc. In some other instances, the term “linker” as used herein may refer to a specifically defined element, such as a linker “capable of being cleaved by Cathepsin B” (as described further below).

The term “cleavable” as used herein, refers to a linker that connects two moieties of the antibody and the antitumor compound by covalent connections, but breaks down to sever the covalent connection between the moieties under physiologically relevant conditions. Cleavage generally releases the antitumor compound from the antibody. The term “cleavable” as used herein, refers to a linker that is not especially susceptible to breaking down under physiological conditions. Such a linker is sufficiently resistant to degradation to keep the antitumor compound connected to the antibody or antigen binding fragment until the antibody or antigen binding fragment is itself at least partially degraded.

The term “peptide” as used herein refers to a compound comprising a continuous sequence of at least two amino acids linked to each other via peptide linkages. The terms “dipeptide”, “tripeptide” and “tetrapeptide” respectively refer to a compound comprising a continuous sequence of two, three and four amino acids linked to each other via peptide linkages. The term “peptide linkage” in this connection is meant to encompass (backbone) amide bonds as well as modified linkages, which can be obtained if non-natural amino acids are introduced in the peptidic sequence. In this case, the modified linkage replaces the (backbone) amide bond which is formed in the continuous peptide sequence by reacting the amino group and the carboxyl group of two amino acid residues. For instance, the modified linkage may be an ester, a thioester, a carbamide, a thiocarbamide or a triazole linkage. Preferably, the amino acids forming the continuous peptide sequence are linked to each other via backbone amide bonds. The peptide may be linear or branched. In preferred aspects, the peptide is a linear di-, tri-, tetra-peptide, more preferably a linear tri- or tetra-peptide.

The term “amino acid” as used herein refers to a compound that contains or is derived from a compound containing at least one amino group and at least one acidic group, preferably a carboxyl group. The distance between amino group and acidic group is not particularly limited. α-, β-, and γ-amino acids are suitable but α-amino acids and especially α-amino carboxylic acids are particularly preferred. The term “amino acid” encompasses both naturally occurring amino acids such as the naturally occurring proteinogenic amino acids, as well as synthetic amino acids that are not found in nature. In the following, a reference to amino acids may be made by means of the 3-letter amino acid code (Arg, Phe, Ala, Cys, Gly, Gln, etc.), or by means of the 1-letter amino acid code (R, F, A, C, G, Q, etc.). Unless specified otherwise, reference to an amino acid by means of the 3-letter amino acid code refers to the corresponding (L)- or (D)-amino acid. Herein, amino acid sequences are written from the N-terminus to the C-terminus (left to right). Unless specified otherwise or dictated otherwise by the context, all connections between adjacent amino acid groups are formed by peptide (amide) bonds.

The term “cycloalkyl group” as used herein refers to a substituted or unsubstituted, cyclic hydrocarbon group having from 3 to 20 carbon atoms, preferably from 5 to 8 carbon atoms. The cycloalkyl group may consist of a single ring, but it may also be formed by two or more condensed rings. Examples of cycloalkyl groups include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl. More preferably, the cycloalkyl group is a cyclopentyl or cyclohexyl group.

The term “alkyl group” as used herein refers to a linear (straight chain) or branched, saturated or unsaturated hydrocarbon group having from 1 to 20 carbon atoms, preferably from 1 to 5 carbon atoms. Examples of alkyl groups include, but are not limited to, -methyl, -ethyl, -n-propyl, -n-butyl, -n-pentyl, -n-hexyl, -n-heptyl, -n-octyl, -isopropyl, -sec-butyl, -isobutyl, -tert-butyl, -isopentyl, -vinyl, -allyl, -1-butenyl, -2-butenyl, -1-pentenyl, -2-pentenyl, -3-methyl-1-butenyl, -2-methyl-2-butenyl, -2,3-dimethyl-2-butenyl, -1-hexyl, 2-hexyl, -3-hexyl, -propynyl, -1-butynyl, -2-butynyl, -1-pentynyl, -2-pentynyl and -3-methyl-1 butynyl. More preferably, the alkyl group is a methyl or an ethyl group.

The term “self-immolative spacer” as used herein refers to a bifunctional chemical moiety which is capable of covalently linking together two spaced chemical moieties into a normally stable tripartite molecule, can release one of the spaced chemical moieties from the tripartite molecule by means of enzymatic cleavage; and following enzymatic cleavage, can spontaneously cleave from the remainder of the molecule to release the other of the spaced chemical moieties.

As used herein, “monosaccharide” refers to any of the class of sugars that cannot be hydrolyzed to give a simpler sugar. Monosaccharides typically are C5 (e.g., xylose) and C6 sugars (e.g., glucose), but may also include monosaccharides having other numbers of carbon, such as C3, C4, C7, C8, and so on. Expressed another way, monosaccharides are the simplest building blocks of oligosaccharides and polysaccharides.

A “disaccharide” herein refers to a carbohydrate having two monosaccharides joined by a glycosidic linkage. An “oligosaccharide” herein refers to a carbohydrate that consists of 2 to 9 monosaccharides, for example, joined by glycosidic linkages. An oligosaccharide can also be referred to herein as an “oligomer”. Monosaccharides that are comprised within a disaccharide or oligosaccharide can be referred to as “monosaccharide units” or “monomeric units”, for example. Preferred monosaccharides herein are fructose and glucose.

As used herein, “oligosaccharide” refers to linear or branched carbohydrate molecules of the same or different monosaccharide units joined together by glycosidic bonds having the general formula of Cₓ(H₂O)_{y}. Oligosaccharides may be thought of as shorter chain polysaccharides, i.e., polysaccharides simply having less monomeric residues in the polymeric chain. When an oligosaccharide contains C6 monosaccharide residues, the general formula may be represented as (C₆H₁₀O₅)n, where n is about 2 to about 9 (i.e., the number of hexose monomers in the oligosaccharide). As used herein, an oligomer (e.g., cello-oligosaccharide) has a DP of 2 to about 9, whereas a polymer (e.g., cellulose) has a DP of at least about 10.

As used herein, “drug” refers to a compound having an antitumor effect (payloads or ADC payloads) and a substituent group or a partial structure allowing connection to a linker structure. When a part or whole linker is cleaved in tumor cells, the drug (i.e., antitumor compound moiety) is released to exhibit the antitumor effect of the antitumor compound. As the linker is cleaved at a connecting position to the drug, the antitumor compound can be released in an unmodified structure to exhibit its intrinsic antitumor effect. For instance, the ideal payloads may have the following characteristics. First, they may have adequately high cytotoxicity. Tumor-specific antigens are very limited, especially in solid tumors. Moreover, due to the low permeability and poor internalization activity of monoclonal antibodies, the number of ADC payloads that can be endocytosed into tumor cells via antibody antigen binding is very low. Second, ADC payloads may have sufficiently low immunogenicity.

As used herein, “topoisomerase 1 inhibitor” refers to a compound inhibiting the activity of topoisomerase 1 which is an important ribozyme for genomic stability and DNA structure preservation, has become a popular target for ADC. Topoisomerase 1 (TOPO-I) inhibitors are associated with innate and adaptive immune responses, suggesting that ADCs targeting TOPO-I may also contribute to antitumor immunotherapy.

As used herein, “hydrophilic appendage” (hydrophilic group or hydrophilic unit) refers to a group or unit which may increase overall aqueous solubility and conjugation efficiency thereby limiting the accumulation and aggregation of the ADC during conjugation as well as in circulation.

As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” cancer may refer to inhibiting growth and/or spread of the cancer cells, killing the cancer cells, or shrinking the cancer cells. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to kidney cancer, spleen cancer, lung cancer, liver cancer, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “effective amount” means an amount of a therapeutic, prophylactic, and/or diagnostic agent that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition.

The terms “subject,” “patient,” “individual,” and the like are used interchangeably herein, and refer to any animal, or cells thereof whether in vitro or in situ, amenable to the methods described herein. In certain non-limiting embodiments, the patient, subject or individual is a human.

### DETAILED DESCRIPTION

### 1. GGYG Linker System

(1) The present disclosure provides a compound of Formula (1):

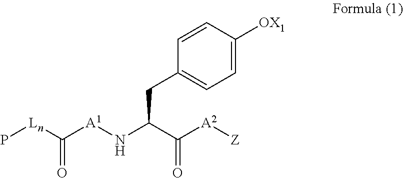

wherein
P is -(maleimide-N)—, -(dibromomaleimide-N)—, -(bromoacetamide-N)—, —(Y—CH₂)—, or -(dibenzocyclooctyne-N—C(═O))—, Y being Br, Cl or I, and the -(dibenzocyclooctyne-N—C(═O))— having the structure of

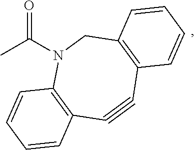 [SMILES: CC(=O)N1Cc2ccccc2C#Cc2ccccc21]

Lₙ is optionally included, and is cycloalkyl, alkyl, or —(OCH₂CH₂)—, —(CH₂—C(═O)—NH—CH₂—CH₂)— if included, and n is an integer of 1 to 6;
A¹ is a peptide residue containing 0-3 amino acids;
A² is a peptide residue containing 0-3 amino acids;
Z is optionally included, and is a self-immolative spacer, —(NHCH₂)—, or p-aminocarbamate if included; and
X₁ is H, monosaccharide, disaccharide, oligosaccharide, polyethylene glycol, sulfate, phosphate, or pyrophosphate.

(2) In addition, the present disclosure provides a linker-drug conjugate comprising:
the compound according to the embodiment mentioned above (1); and
a drug which is conjugated to the compound via Z of Formula (1).

In one embodiment, the drug (payloads or ADC payloads) may include (i) microtubules targeting payloads such as maytansinoids, auristatin, eribulin, tubulysins, cryptophycins, and EG5 inhibitors, (ii) DNA targeting payloads such as enediyne, topoisomerase 1 inhibitors, pyrrolo[2,1-c][1,4] benzodiazepines (PBD), and duocarmycins, (iii) RNA targeting payloads such as thailanstatin, and amatoxins, (iv) immune ADC payloads such as toll-like receptor agonists, the stimulator of interferon genes (STING), glucocorticoid receptor modulators, and (v) any novel potential ADC payloads such as Bcl-xL inhibitors, Niacinamide phosphate ribose transferase (NAMPT), Carmaphycins, PROTAC molecules, Near-infrared photoimmunotherapy (NIR-PIT) drugs, and dual payloads (e.g., MMAE and MMAF).

(3) The present disclosure also provides the linker-drug conjugate according to the embodiment mentioned above (2), wherein the drug is conjugated to the compound in the form of —(NH-drug)- or —(O-drug)-.

(4) The present disclosure also provides the linker-drug conjugate according to the embodiment mentioned above (2), wherein the drug is a topoisomerase I inhibitor.

The “topoisomerase 1 inhibitor” refers to a compound inhibiting the activity of topoisomerase 1 which is an important ribozyme for genomic stability and DNA structure preservation, has become a popular target for ADC. Topoisomerase 1 (TOPO-I) inhibitors are associated with innate and adaptive immune responses, suggesting that ADCs targeting TOPO-I may also contribute to antitumor immunotherapy.

For instance, the linker-drug conjugate may include the following compounds.

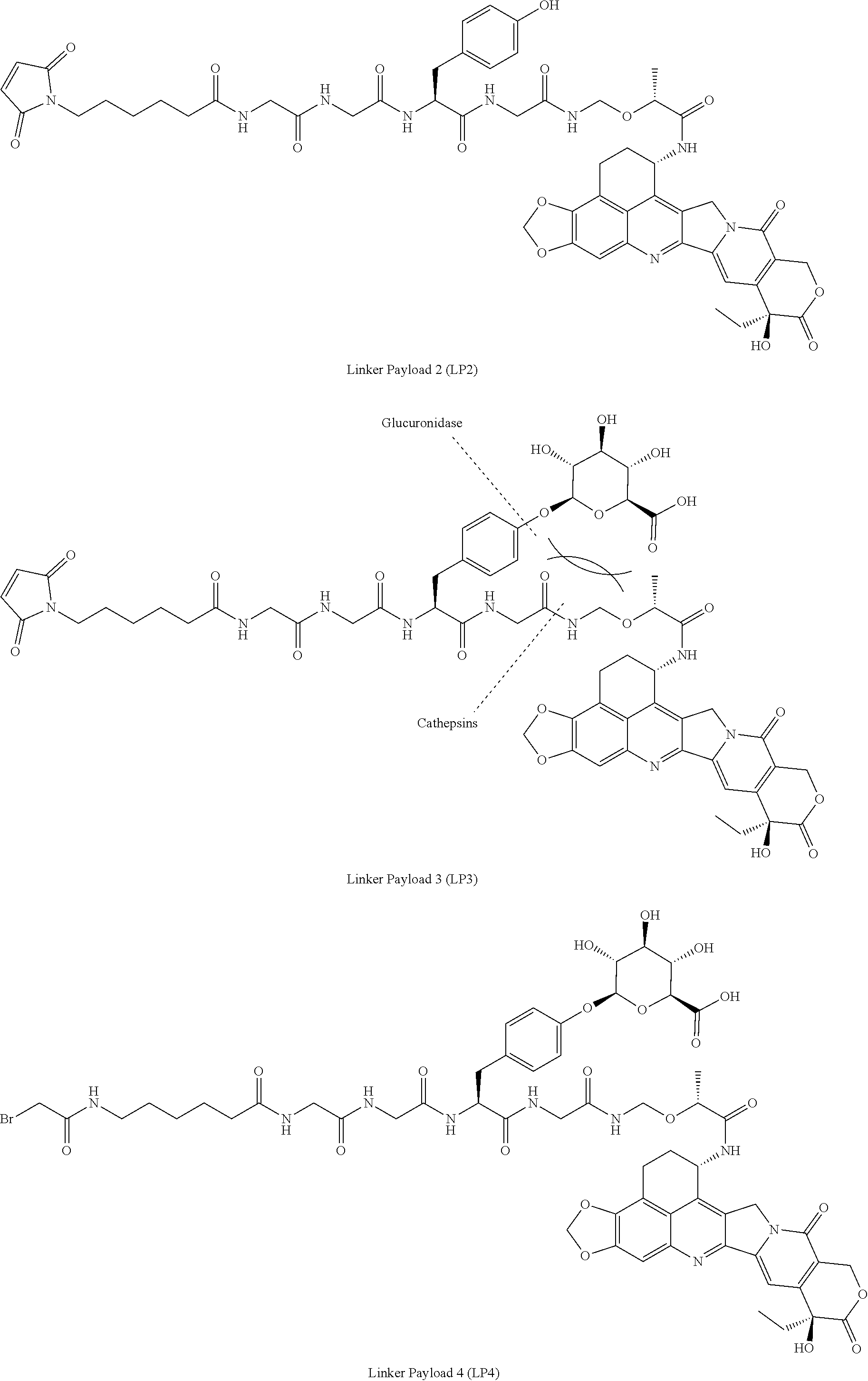

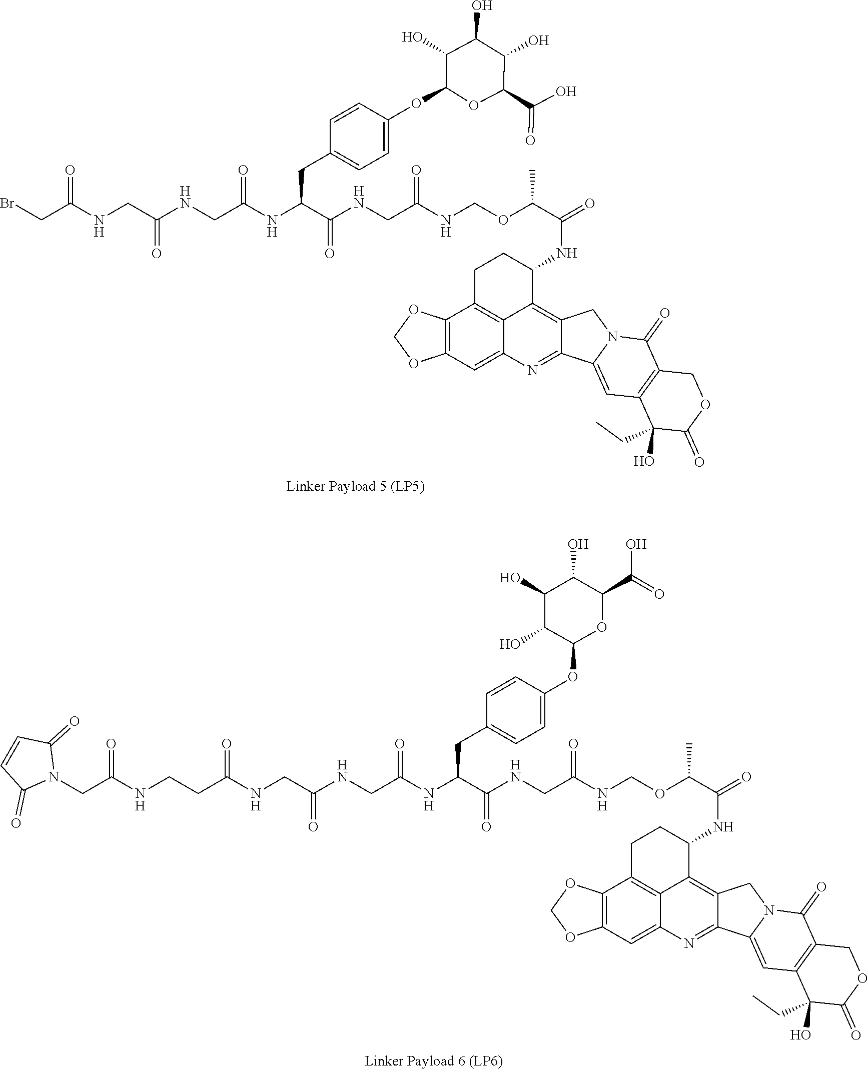 [SMILES: CC[C@@]1(O)C(=O)OCc2c1cc1n(c2=O)Cc2c-1nc1cc3c(c4c1c2[C@@H](NC(=O)[C@@H](C)OCNC(=O)CNC(=O)[C@H](Cc1ccc(O[C@@H]2O[C@H](C(=O)O)[C@@H](O)[C@H](O)[C@H]2O)cc1)NC(=O)CNC(=O)CNC(=O)CBr)CC4)OCO3 ; CC[C@@]1(O)C(=O)OCc2c1cc1n(c2=O)Cc2c-1nc1cc3c(c4c1c2[C@@H](NC(=O)[C@@H](C)OCNC(=O)CNC(=O)[C@H](Cc1ccc(O[C@@H]2O[C@H](C(=O)O)[C@@H](O)[C@H](O)[C@H]2O)cc1)NC(=O)CNC(=O)CNC(=O)CCNC(=O)CN1C(=O)C=CC1=O)CC4)OCO3]

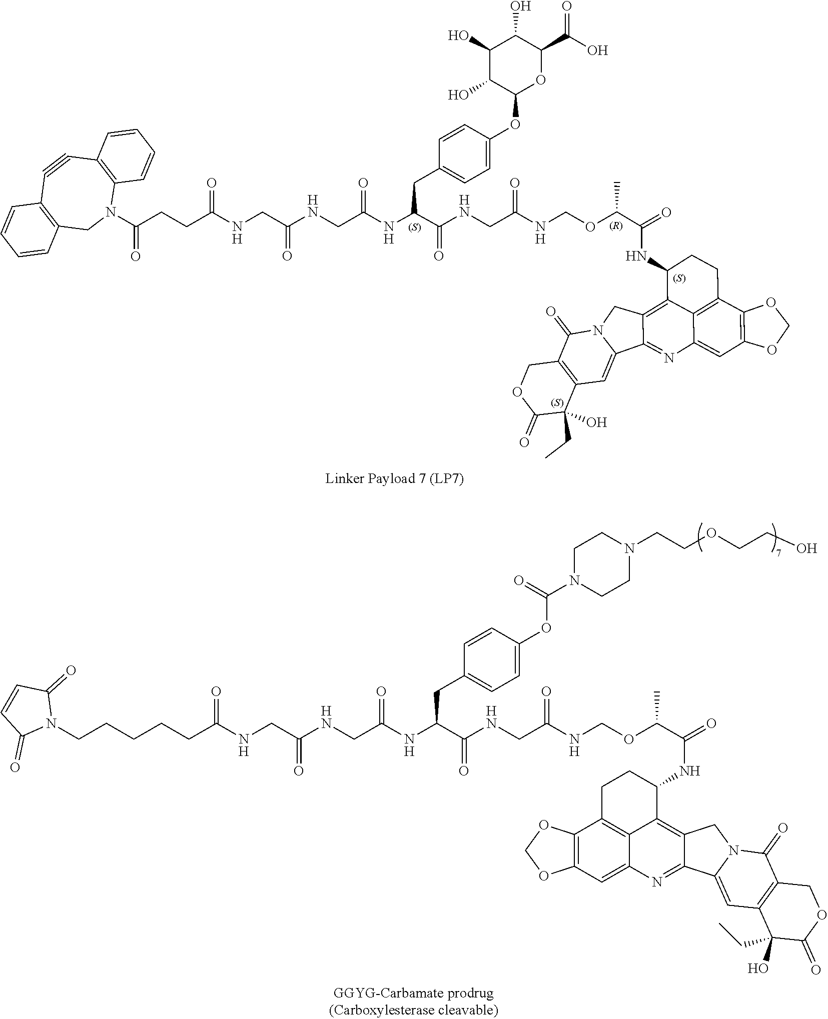

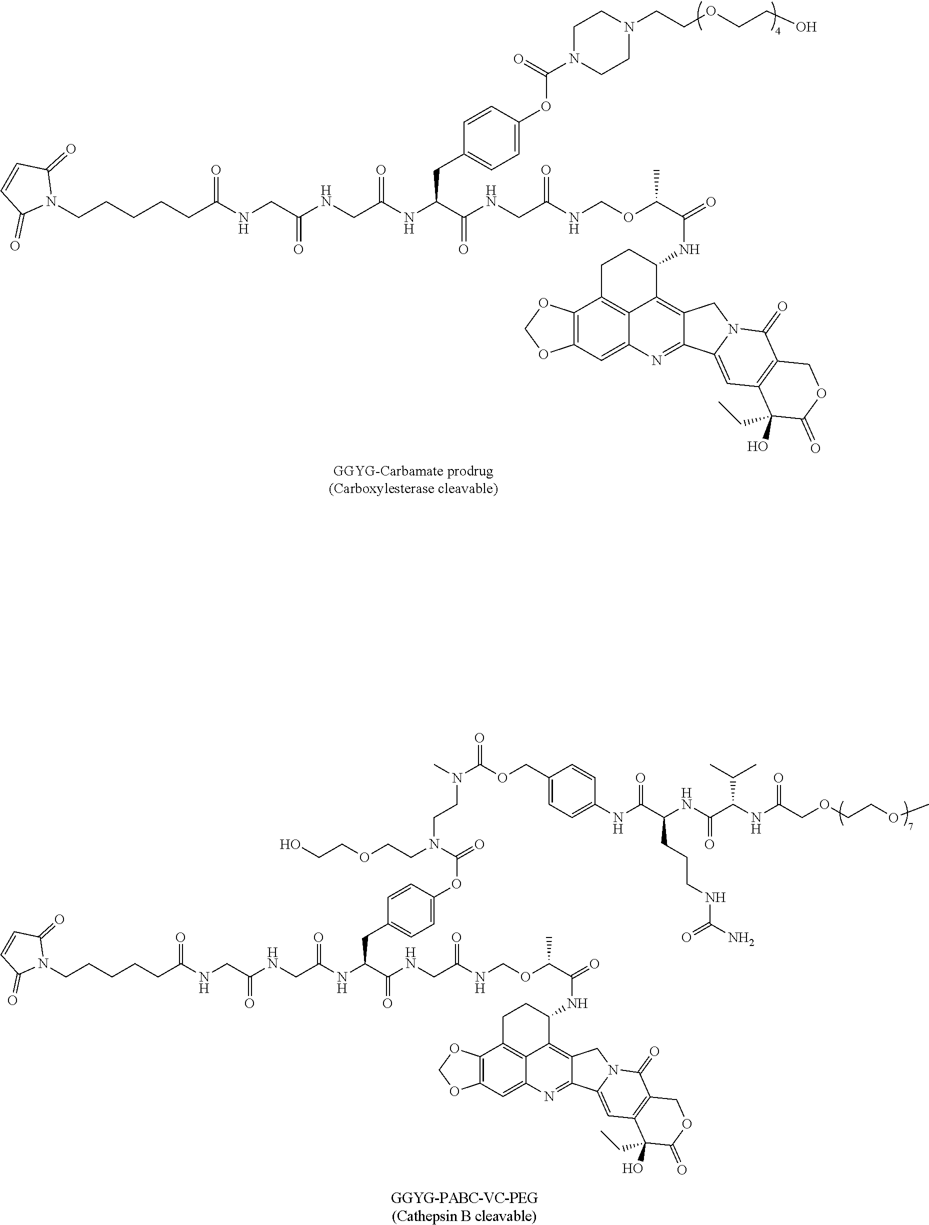

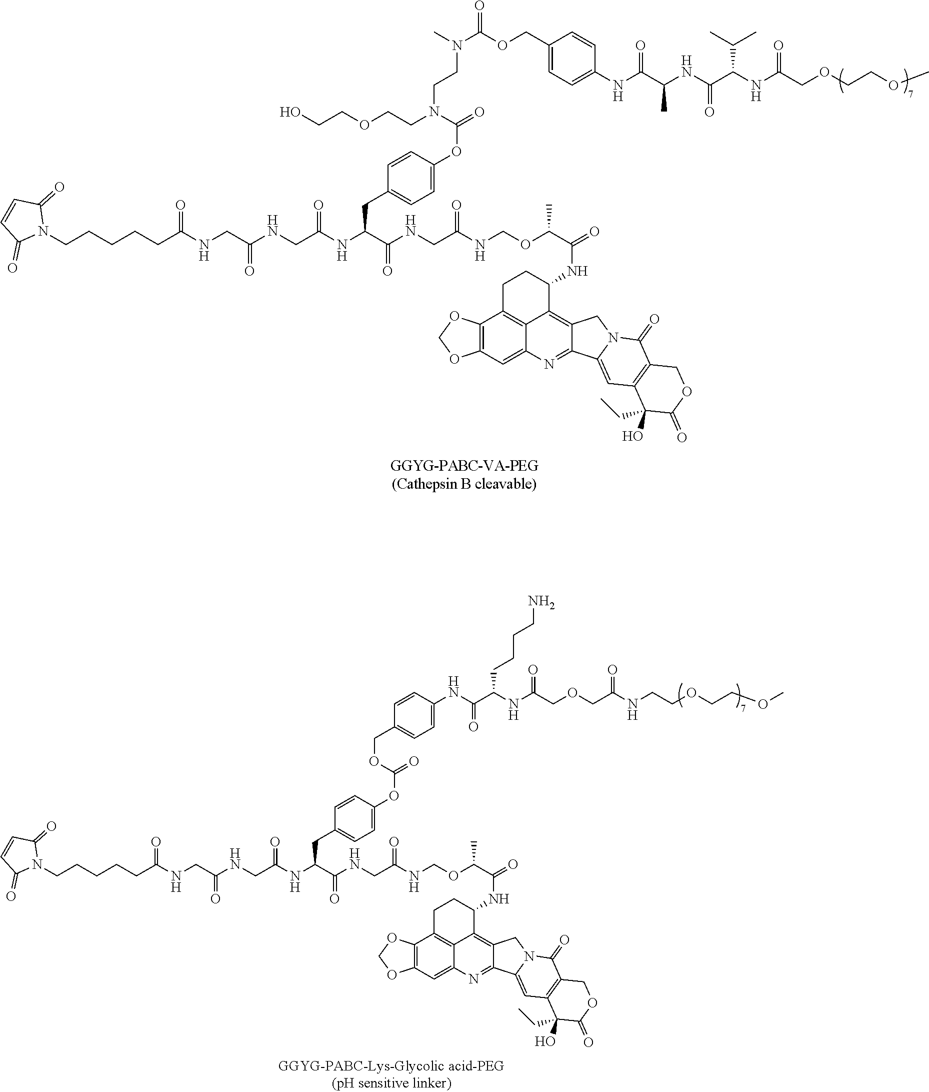

(5) The present disclosure provides an antibody-drug conjugate comprising an antibody or an antigen binding fragment thereof, a drug, and a linker,
wherein the linker is the compound according to the embodiment mentioned above (1),
wherein the antibody or the antigen binding fragment thereof is conjugated to the compound via P of Formula (1), and
wherein the drug is conjugated to the compound via Z of Formula (1).

In one embodiment, the antigen binding fragment thereof may comprise one or more of a Fab fragment, a Fab′ fragment, a Fab′-SH, a Fv fragment, a scFv fragment, a F(ab′)₂ fragment, a VL fragment, a VH fragment, a ScFv-Fc fragment, and a (ScFv)₂-Fc fragment, a diabody, a linear antibody, a fragment produced by a Fab expression library, an anti-idiotypic (anti-Id) antibody, a complementary determining region (CDR), and an epitope-binding fragment.

(6) The present disclosure provides a method of treating cancer, the method comprising administering an effective amount of the antibody-drug conjugate of the embodiment mentioned above (5) to a subject in need thereof.

In one embodiment, the antibody-drug conjugate can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a cancer, such as a blood cancer or a solid tumor.

The effective amount can be administered in one or more administrations, applications or dosages and is not intended to be limited to a particular formulation or administration route. In an embodiment, administering is via a course of treatment comprising a plurality of treatment cycles and a plurality of rest periods.

In another embodiment, the antibody-drug conjugate may be administered to the subject at a concentration which is efficient to treat cancer of the subject. For instance, the antibody-drug conjugate may be in the range of 1 mg/kg to 4-5 mg/kg of body weight.

(7) The present disclosure provides the method of the embodiment mentioned above (6), wherein the cancer comprises one or more of breast cancer, liver cancer, skin cancer, ovarian cancer, cervical cancer, prostate cancer, testicular cancer, brain cancer, clear cell renal cell carcinoma, glioma, melanoma, lung cancer, non-small cell lung cancer (NSCLC), small cell lung cancer, pancreatic cancer, gastric cancer, acute myeloid leukemia (AML), Hodgkin's lymphoma, non-Hodgkin's lymphoma (NHL), colorectal cancer, colon cancer, renal cancer, esophageal cancer, leukaemia, hepatocellular carcinoma, bone cancer, bladder cancer, sarcomas, kidney cancer, head and neck cancer, hypopharyngeal squamous cell carcinoma, glioblastoma, neuroblastoma, endometrial cancer, and urothelial cell carcinoma.

### 2. DGGFG or GGGFG Linker System

(8) The present disclosure also provides a compound of Formula (2):

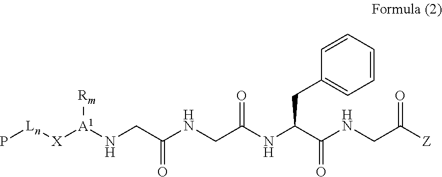

wherein
P is -(maleimide-N)—, -(dibromomaleimide-N)—, -(bromoacetamide-N)—, or -(dibenzocyclooctyne-N—C(═O))—;
Lₙ is optionally included, and is cycloalkyl, alkyl or a direct bond if included, and n is an integer of 1 to 6;
X is —(C═O)—, a direct bond, or a direct bond to a side chain of A¹;
A¹ is an amino acid;
Rₘ is a hydrophilic appendage attached to A¹ and m represents an integer of 1 to 10; and
Z is optionally included, and is a self-immolative spacer, —(NHCH₂)—, or p-aminocarbamate if included.

In one embodiment, the “hydrophilic appendage” refers to a group or unit which may increase overall aqueous solubility and conjugation efficiency thereby limiting the accumulation and aggregation of the ADC during conjugation as well as in circulation.

In one embodiment, the hydrophilic appendage may be selected from the following compounds.

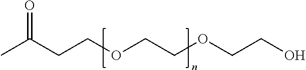

Here, n may be an integer in the range of 3-24. In one embodiment, the lower limit of n may be 4, 5, 6, 7, 8, 9, 10, 11, 12, 13, 14, 15, 16, 17, 18, 19, 20, 21, 22. In one embodiment, the upper limit of n may be 23, 22, 21, 20, 19, 18, 17, 16, 15, 14, 13, 12, 10, 9, 8, 7, 6, 5 or 4. In one embodiment, n may be 3, 4, 5, 6, 7, 8, 9, 10, 11, 12, 13, 14, 15, 16, 17, 18, 19, 20, 21, 22, 23 or 24.

(9) The present disclosure provides the compound according to the embodiment mentioned above (8), wherein the amino acid is aspartic acid, glycine, glutamic acid or lysine.

(10) The present disclosure provides a linker-drug conjugate comprising:
the compound according to the embodiment mentioned above (8); and
a drug which is conjugated to the compound via Z of Formula (2).

In one embodiment, the drug (payloads or ADC payloads) may include (i) microtubules targeting payloads such as maytansinoids, auristatin, eribulin, tubulysins, cryptophycins, and EG5 inhibitors, (ii) DNA targeting payloads such as enediyne, topoisomerase 1 inhibitors, pyrrolo[2,1-c][1,4] benzodiazepines (PBD), and duocarmycins, (iii) RNA targeting payloads such as thailanstatin, and amatoxins, (iv) immune ADC payloads such as toll-like receptor agonists, the stimulator of interferon genes (STING), glucocorticoid receptor modulators, and (v) any novel potential ADC payloads such as Bcl-xL inhibitors, Niacinamide phosphate ribose transferase (NAMPT), Carmaphycins, PROTAC molecules, Near-infrared photoimmunotherapy (NIR-PIT) drugs, and dual payloads (e.g., MMAE and MMAF).

(11) The present disclosure provides the linker-drug conjugate according to the embodiment mentioned above (10), wherein the drug is conjugated to the compound in the form of —(NH-drug)- or —(O-drug)-.

(12) The present disclosure provides the linker-drug conjugate according to the embodiment mentioned above (10), wherein the drug is a topoisomerase 1 inhibitor.

The “topoisomerase 1 inhibitor” refers to a compound inhibiting the activity of topoisomerase 1 which is an important ribozyme for genomic stability and DNA structure preservation, has become a popular target for ADC. Topoisomerase 1 (TOPO-I) inhibitors are associated with innate and adaptive immune responses, suggesting that ADCs targeting TOPO-I may also contribute to antitumor immunotherapy.

For instance, the linker-drug conjugate may include the following compounds.

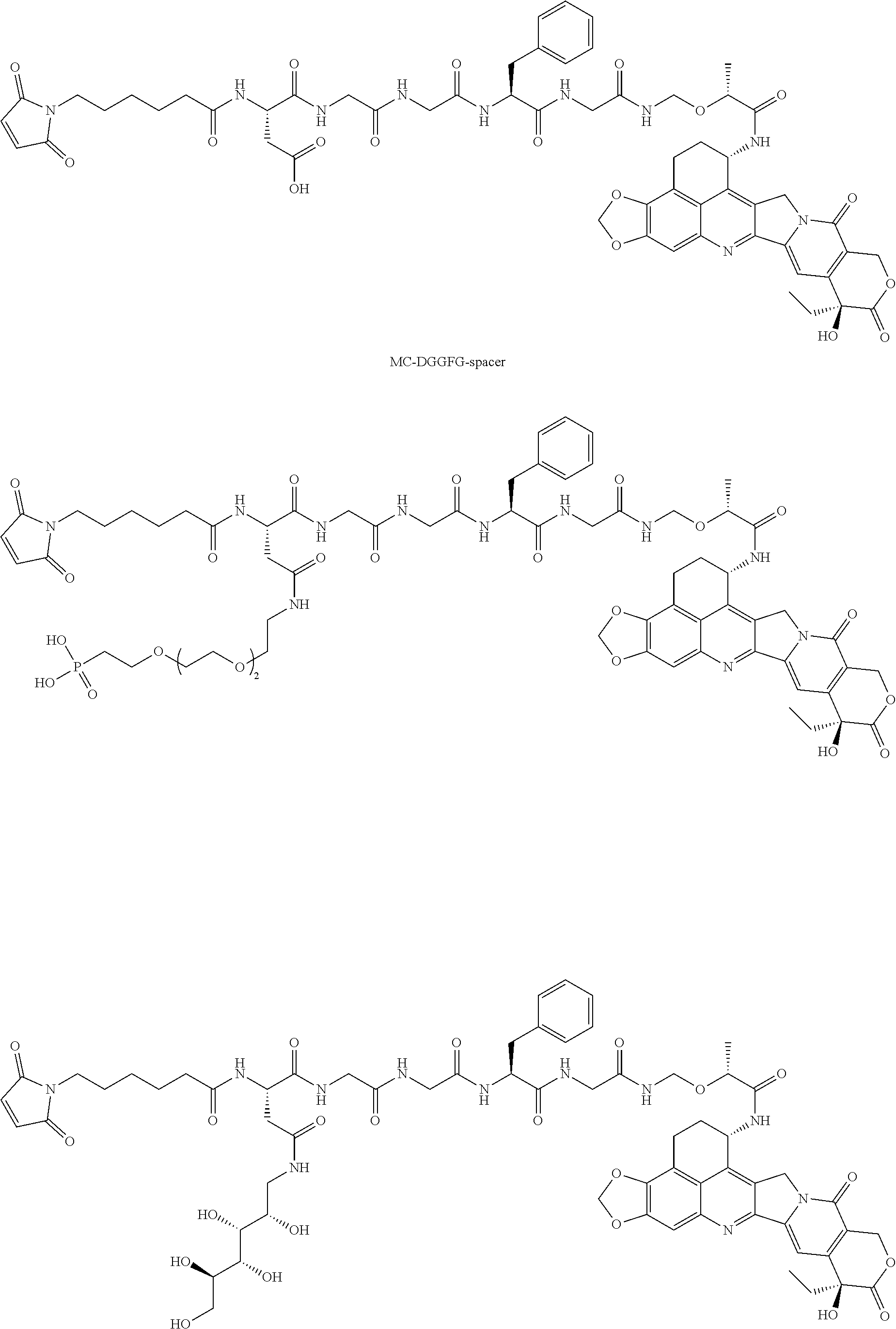

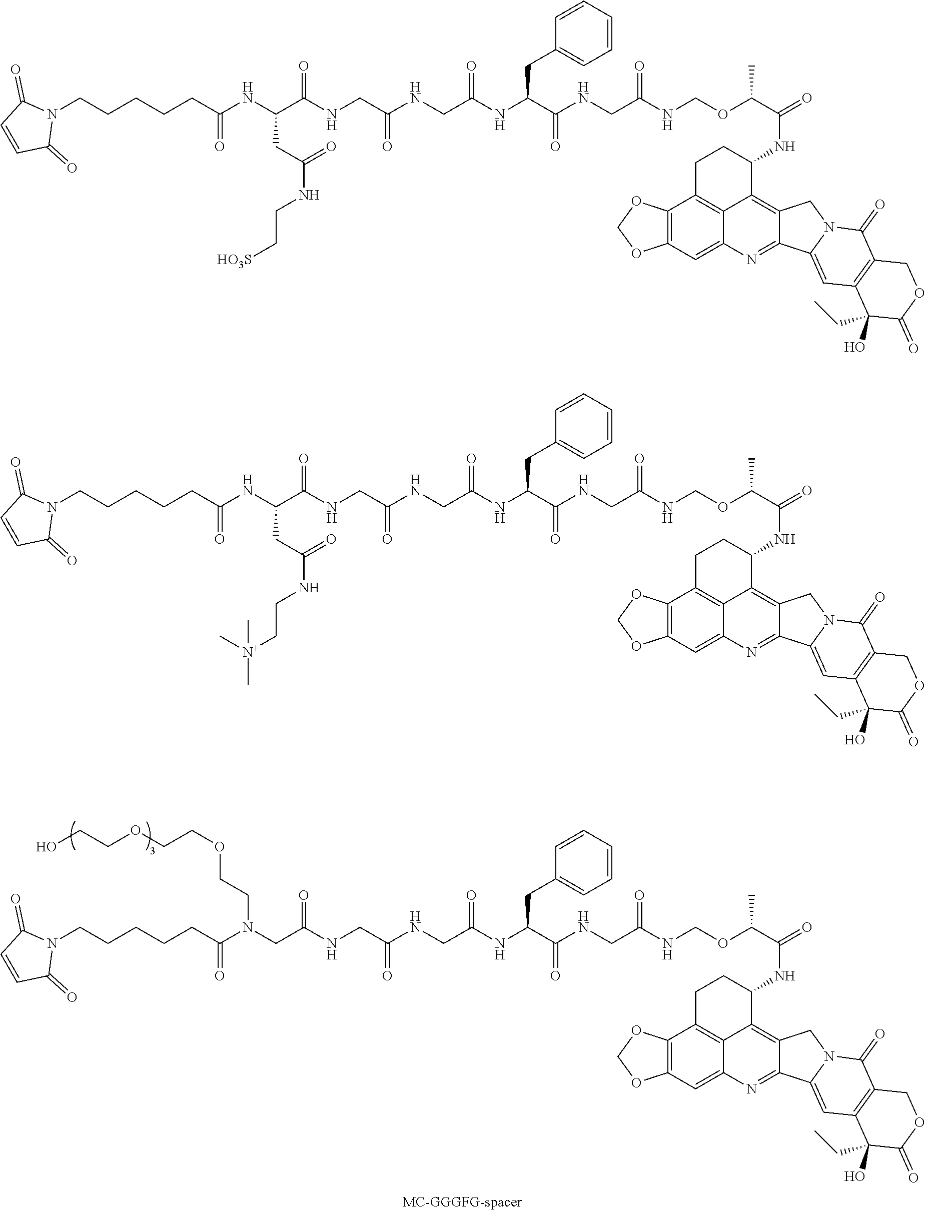

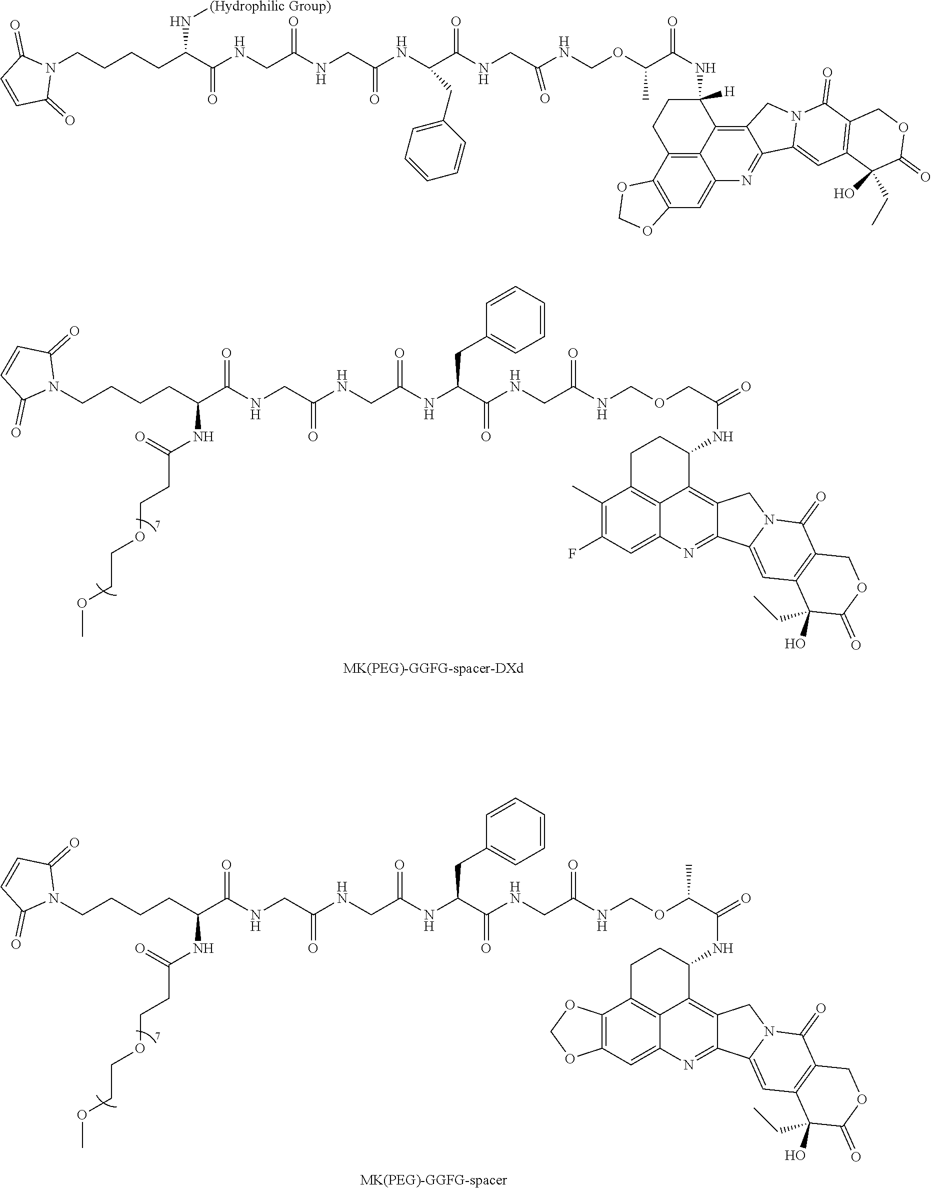

(13) The present disclosures provides an antibody-drug conjugate comprising an antibody or an antigen binding fragment thereof, a drug, and a linker,
wherein the linker is the compound according to the embodiment mentioned above (8),
wherein the antibody or the antigen binding fragment thereof is conjugated to the compound via P of Formula (2), and
wherein the drug is conjugated to the compound via Z of Formula (2).

(14) The present disclosure provides a method of treating cancer, the method comprising administering an effective amount of the antibody-drug conjugate of the embodiment mentioned above (13) to a subject in need thereof.

In one embodiment, the antibody-drug conjugate can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a cancer, such as a blood cancer or a solid tumor.

The effective amount can be administered in one or more administrations, applications or dosages and is not intended to be limited to a particular formulation or administration route. In an embodiment, administering is via a course of treatment comprising a plurality of treatment cycles and a plurality of rest periods.

In another embodiment, the antibody-drug conjugate may be administered to the subject at a concentration which is efficient to treat cancer of the subject. For instance, the antibody-drug conjugate may be in the range of 1 mg/kg to 4-5 mg/kg of body weight.

(15) The present disclosure provides the method of the embodiment mentioned above (14), wherein the cancer comprises one or more of breast cancer, liver cancer, skin cancer, ovarian cancer, cervical cancer, prostate cancer, testicular cancer, brain cancer, clear cell renal cell carcinoma, glioma, melanoma, lung cancer, non-small cell lung cancer (NSCLC), small cell lung cancer, pancreatic cancer, gastric cancer, acute myeloid leukemia (AML), Hodgkin's lymphoma, non-Hodgkin's lymphoma (NHL), colorectal cancer, colon cancer, renal cancer, esophageal cancer, leukaemia, hepatocellular carcinoma, bone cancer, bladder cancer, sarcomas, kidney cancer, head and neck cancer, hypopharyngeal squamous cell carcinoma, glioblastoma, neuroblastoma, endometrial cancer, and urothelial cell carcinoma.

### 3. GGFG Linker System

(16) The present disclosure also provides a compound of Formula (3):

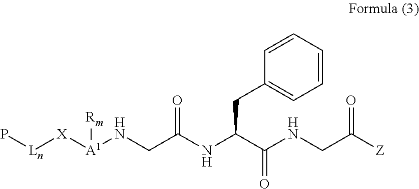

wherein
P is -(maleimide-N)—, -(dibromomaleimide-N)—, -(bromoacetamide-N)—, or -(dibenzocyclooctyne-N—C(═O))—;
Lₙ is optionally included, and is cycloalkyl, alkyl or a direct bond if included, and n is an integer of 1 to 6;
X is —(C═O)—, or a direct bond;
A¹ is an amino acid;
Rₘ is optionally included, and is a hydrophilic appendage attached to A¹ if included, and m represents an integer of 1 to 10; and
Z is optionally included, and is a self-immolative spacer, —(NHCH₂)—, or p-aminocarbamate if included.

In one embodiment, the “hydrophilic appendage” refers to a group or unit which may increase overall aqueous solubility and conjugation efficiency thereby limiting the accumulation and aggregation of the ADC during conjugation as well as in circulation.

In one embodiment, the hydrophilic appendage may be selected from the following compounds.

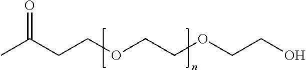

Here, n may be an integer in the range of 3-24. In one embodiment, the lower limit of n may be 4, 5, 6, 7, 8, 9, 10, 11, 12, 13, 14, 15, 16, 17, 18, 19, 20, 21, 22. In one embodiment, the upper limit of n may be 23, 22, 21, 20, 19, 18, 17, 16, 15, 14, 13, 12, 10, 9, 8, 7, 6, 5 or 4. In one embodiment, n may be 3, 4, 5, 6, 7, 8, 9, 10, 11, 12, 13, 14, 15, 16, 17, 18, 19, 20, 21, 22, 23 or 24.

(17) The present disclosure provides the compound according to the embodiment mentioned above (16), wherein the amino acid is aspartic acid, glycine, glutamic acid or lysine.

(18) The present disclosure provides a linker-drug conjugate comprising:
the compound according to the embodiment mentioned above (16); and
a drug which is conjugated to the compound via Z of Formula (2).

In one embodiment, the drug (payloads or ADC payloads) may include (i) microtubules targeting payloads such as maytansinoids, auristatin, eribulin, tubulysins, cryptophycins, and EG5 inhibitors, (ii) DNA targeting payloads such as enediyne, topoisomerase 1 inhibitors, pyrrolo[2,1-c][1,4] benzodiazepines (PBD), and duocarmycins, (iii) RNA targeting payloads such as thailanstatin, and amatoxins, (iv) immune ADC payloads such as toll-like receptor agonists, the stimulator of interferon genes (STING), glucocorticoid receptor modulators, and (v) any novel potential ADC payloads such as Bcl-xL inhibitors, Niacinamide phosphate ribose transferase (NAMPT), Carmaphycins, PROTAC molecules, Near-infrared photoimmunotherapy (NIR-PIT) drugs, and dual payloads (e.g., MMAE and MMAF).

(19) The present disclosure provides the linker-drug conjugate according to the embodiment mentioned above (18), wherein the drug is conjugated to the compound in the form of —(NH-drug)- or —(O-drug)-.

(20) The present disclosure provides the linker-drug conjugate according to the embodiment mentioned above (18), wherein the drug is a topoisomerase 1 inhibitor.

For instance, the linker-drug conjugate may include the following compounds.

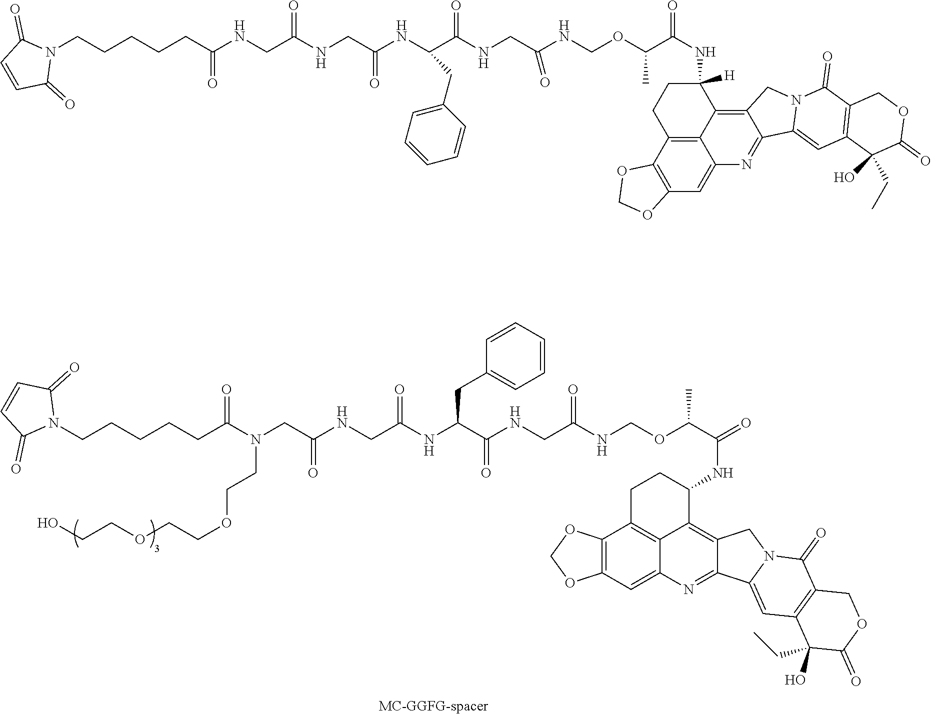

(21) The present disclosure provides an antibody-drug conjugate comprising an antibody or an antigen binding fragment thereof, a drug, and a linker,
wherein the linker is the compound according to the embodiment mentioned above (16),
wherein the antibody or the antigen binding fragment thereof is conjugated to the compound via P of Formula (3), and
wherein the drug is conjugated to the compound via Z of Formula (3).

(22) The present disclosure provides a method of treating cancer, the method comprising administering an effective amount of the antibody-drug conjugate of the embodiment mentioned above (21) to a subject in need thereof.

In one embodiment, the antibody-drug conjugate can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a cancer, such as a blood cancer or a solid tumor.

The effective amount can be administered in one or more administrations, applications or dosages and is not intended to be limited to a particular formulation or administration route. In an embodiment, administering is via a course of treatment comprising a plurality of treatment cycles and a plurality of rest periods.

In another embodiment, the antibody-drug conjugate may be administered to the subject at a concentration which is efficient to treat cancer of the subject. For instance, the antibody-drug conjugate may be in the range of 1 mg/kg to 4-5 mg/kg of body weight.

(23) The present disclosure provides the method of the embodiment mentioned above (22), wherein the cancer comprises one or more of breast cancer, liver cancer, skin cancer, ovarian cancer, cervical cancer, prostate cancer, testicular cancer, brain cancer, clear cell renal cell carcinoma, glioma, melanoma, lung cancer, non-small cell lung cancer (NSCLC), small cell lung cancer, pancreatic cancer, gastric cancer, acute myeloid leukemia (AML), Hodgkin's lymphoma, non-Hodgkin's lymphoma (NHL), colorectal cancer, colon cancer, renal cancer, esophageal cancer, leukaemia, hepatocellular carcinoma, bone cancer, bladder cancer, sarcomas, kidney cancer, head and neck cancer, hypopharyngeal squamous cell carcinoma, glioblastoma, neuroblastoma, endometrial cancer, and urothelial cell carcinoma.

### 4. Other Linker System

The present disclosure also provides the linker systems as follows. For instance, the linker-drug conjugate may include the following compounds.

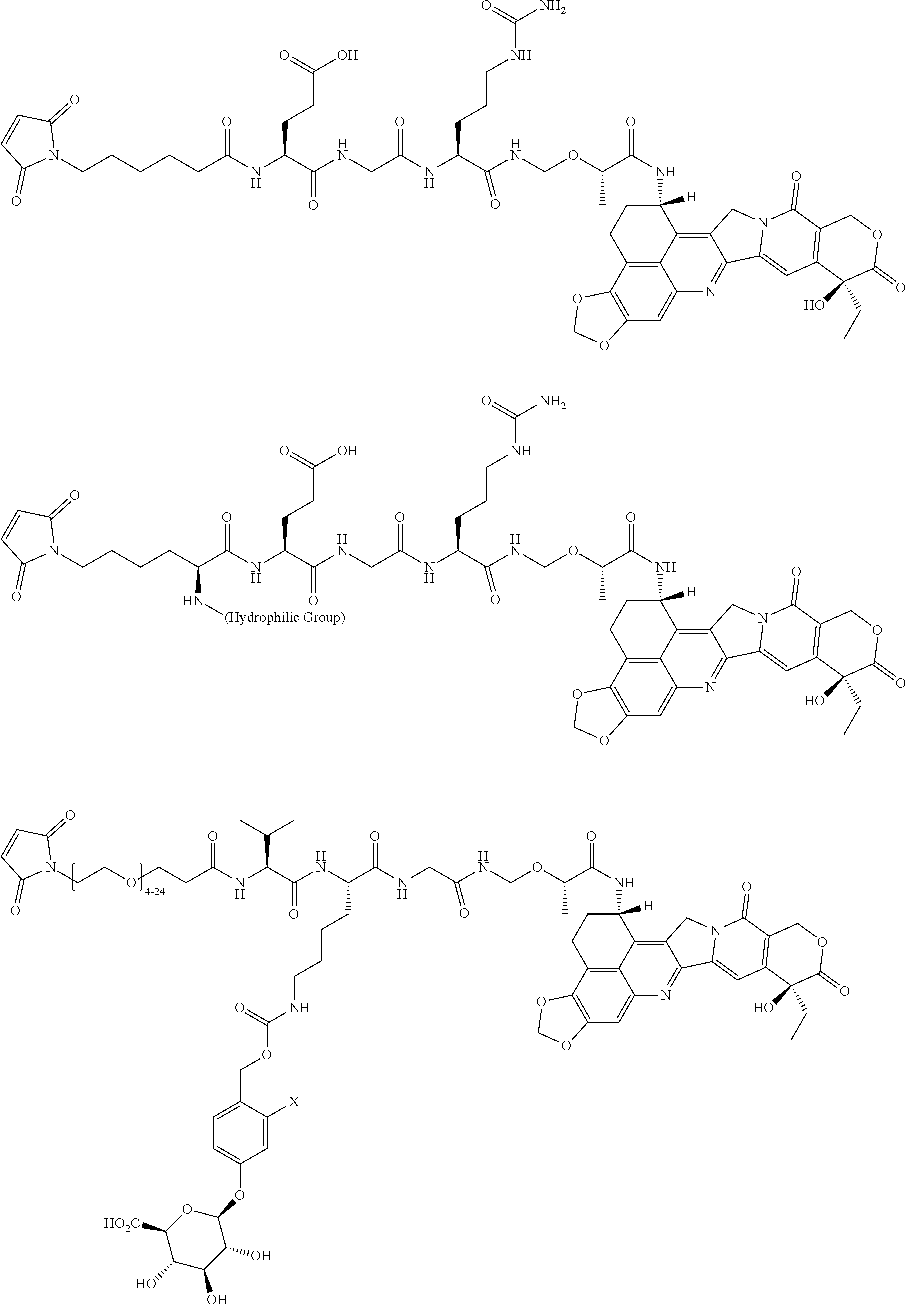

In the above compounds, the hydrophilic group may be as follows.

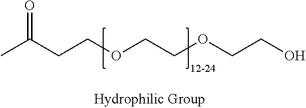

In the embodiments explained in the present disclosure, the specific drug (payload (PBX-7016)) has been conjugated to the various linker systems. However, as discussed in the present disclosure, any drug (payload) which is used for ADC may be conjugated to such linkers. For instance, the drug (payloads or ADC payloads) may include (i) microtubules targeting payloads such as maytansinoids, auristatin, eribulin, tubulysins, cryptophycins, and EG5 inhibitors, (ii) DNA targeting payloads such as enediyne, topoisomerase 1 inhibitors, pyrrolo[2,1-c][1,4] benzodiazepines (PBD), and duocarmycins, (iii) RNA targeting payloads such as thailanstatin, and amatoxins, (iv) immune ADC payloads such as toll-like receptor agonists, the stimulator of interferon genes (STING), glucocorticoid receptor modulators, and (v) any novel potential ADC payloads such as Bcl-xL inhibitors, Niacinamide phosphate ribose transferase (NAMPT), Carmaphycins, PROTAC molecules, Near-infrared photoimmunotherapy (NIR-PIT) drugs, and dual payloads (e.g., MMAE and MMAF).

In addition, any antibody or antigen binding fragment thereof, or any moiety binding to a specific target in the field of the antibody-drug conjugate or relating art may be conjugated to the linkers discussed in this disclosure.

### 5. Pharmaceutical Composition Comprising the Antibody-Drug Conjugate

The antibody-drug conjugate of the present disclosure may be prepared in the form of a pharmaceutical composition comprising the antibody-drug conjugate of the present disclosure and a pharmaceutically acceptable carrier. The pharmaceutically acceptable carrier may be selected based on the particular antitumor compound used, and its concentration, stability and intended bioavailability, the disease, disorder or condition being treated with the composition, the subject, its age, size and general condition, and the route of administration. The antibody-drug conjugate of the present disclosure may be mixed with a solvent such as a sterilized liquid (including water and oil (oil derived from petroleum, animals, vegetables, or synthetic oil (e.g., peanut oil, soybean oil, mineral oil, sesame oil, etc.)), a saline, a dextrose aqueous solution, or a glycerol aqueous solution, and additives such as a moisturizer, an emulsifier, or a pH buffer, and the like, so as to prepare the pharmaceutical composition of the present disclosure. The pharmaceutically acceptable carrier for solid dosage forms may include sugars, starches, and other conventional substances including polysorbate, histidine, lactose, talc, sucrose, gelatin, carboxymethylcellulose, agar, mannitol, sorbitol, calcium phosphate, calcium carbonate, sodium carbonate, kaolin, alginic acid, acacia, corn starch, potato starch, sodium saccharin, magnesium carbonate, tragacanth, microcrystalline cellulose, colloidal silicon dioxide, croscarmellose sodium, talc, magnesium stearate, and stearic acid. In addition, such solid dosage forms may be uncoated or may be coated by known techniques (e.g., to delay disintegration and absorption). In addition, the pharmaceutically acceptable carrier used in formulating liquid dosage forms for oral or parenteral administration, for instance, includes nonaqueous, pharmaceutically-acceptable polar solvents such as oils, alcohols, amides, esters, ethers, ketones, hydrocarbons and mixtures thereof, as well as water, saline solutions, dextrose solutions, electrolyte solutions, or any other aqueous, pharmaceutically acceptable liquid.

### EXAMPLES

### (1) Hydrophilic Groups Importance

The incorporation of hydrophilic groups into the linker structure of Antibody-Drug Conjugates (ADCs) holds vital importance in mitigating potential side effects. This strategic addition serves several crucial roles in enhancing the safety and efficacy profile of ADCs:
i. Reduced Non-Selective Cellular Uptake: Hydrophilic groups contribute to increased water solubility, making it challenging for the ADC to penetrate cell walls, minimizing the unintended exposure of cytotoxic payload in normal cells.
ii. Enhanced Stability in Circulation: Hydrophilic linkers can enhance the stability of ADCs which is crucial for maintaining the integrity of the ADC structure until it reaches the tumor site, preventing systemic release.
iii. Minimized Off-Target Toxicities: By reducing non-specific interactions with normal cells, hydrophilic linkers help minimize off-target toxicities and preserve the therapeutic window of ADCs.
iv. Improved Pharmacokinetics: Hydrophilic modifications can influence the pharmacokinetics of ADCs, optimizing factors such as circulation time and distribution.

### Design: GGFG to GGYG

To increase the tumor specificity and tolerability, stable cleavable linkers are required. The inventors have designed a novel peptide sequence which can be cleaved by the lysosomal enzymes (cathepsins) with similar efficiency as GGFG linker system, along with the provision for introduction of functional moieties to modulate the physicochemical properties of the ADC.

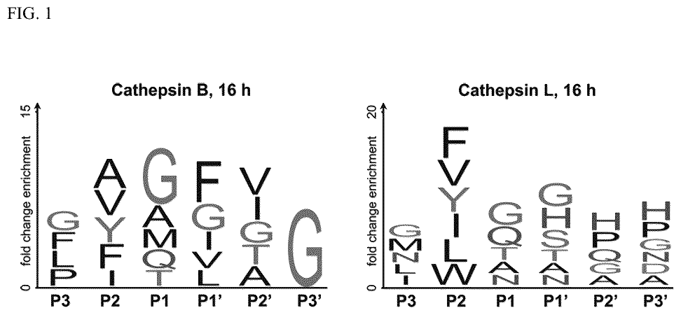

In this regard, the inventors examined the peptide substrate recognition of cathepsins B and L proteases. (FIG. 1) These proteases, characterized by broad substrate specificity, tend to prefer structurally related amino acids in a specific subsite. Taking this into consideration, the inventors designed a novel tetrapeptide sequence by replacing the aromatic amino acid phenyl alanine (F) in GGFG linker system (LP1) with structurally similar amino acid tyrosine(Y).

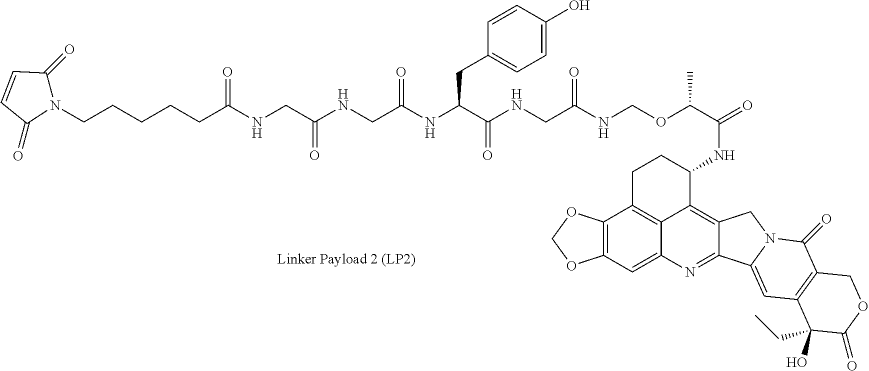 [SMILES: CC[C@@]1(O)C(=O)OCc2c1cc1n(c2=O)Cc2c-1nc1cc3c(c4c1c2[C@@H](NC(=O)[C@@H](C)OCNC(=O)CNC(=O)[C@H](Cc1ccc(O)cc1)NC(=O)CNC(=O)CNC(=O)CCCCCN1C(=O)C=CC1=O)CC4)OCO3]

### Conjugation:

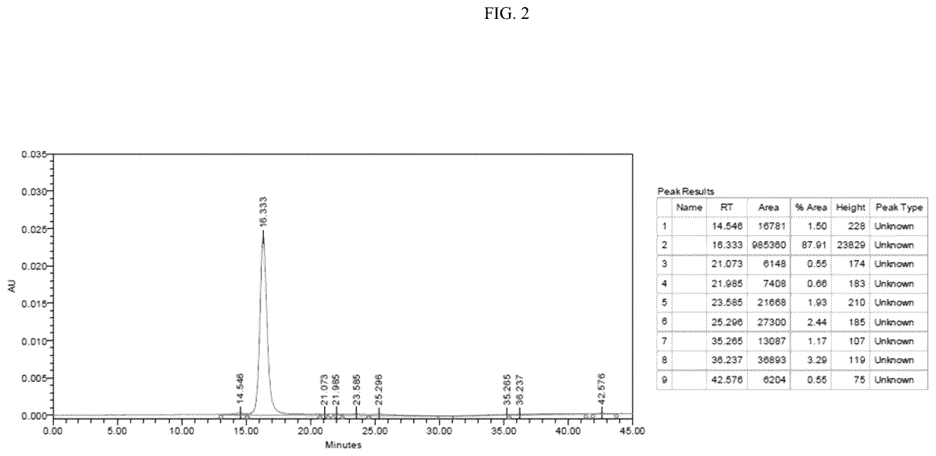

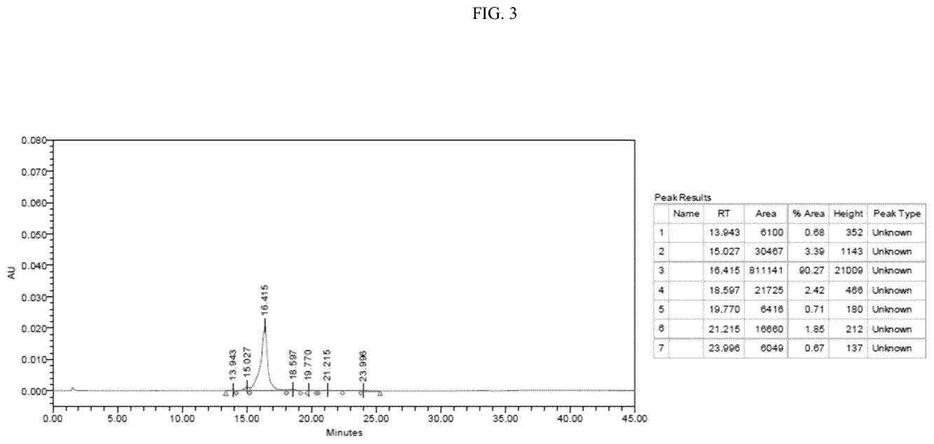

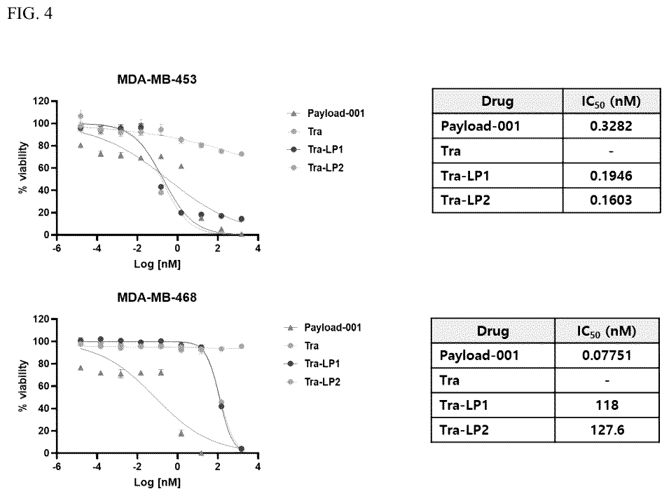

4 mg/ml of Trastuzumab was reacted with 30-fold molar excess of TCEP (Tris(2-carboxyethyl)phosphine hydrochloride) for 2 hrs at 25° C. to generate cysteine from disulfide bonds. Reduced Trastuzumab was purified from unreacted TCEP using PD-10 desalting column. 2 mg/ml of reduced Trastuzumab was reacted with 12-fold molar excess of linker-payload 2 (LP2) under 10% (v/v0) DMSO for 1 hour at 25° C. Trastuzumab ADC was purified from unreacted linker-payloads and DMSO using PD-10 desalting column. See FIGS. 2 to 4.

### (2) Increasing Hyrophilicity and Tandem Cleavage

The hydroxyl group on tyrosine (Y) acts as an attachment point for the hydrophilic pendant groups like saccharides, sulfates, phosphates, PEG linkers etc. Research has shown that elevated enzyme levels of β-glucuronidase have been reported in tumor tissue. The enzyme is found at very low concentrations in the circulation, but is present in high concentrations within solid tumors, including those affecting lung, breast, pancreatic, colorectal, and ovarian cancers.

Exploiting the high prevalence of lysosomal enzyme β-glucuronidase in malignant cells, the inventors have designed a tandem cleavage linker having β-glucuronic acid attached to the hydroxyl group of the tyrosine in the GGYG linker system. This linker is designed to undergo sequential enzymatic cleavage in a specific manner and controlled fashion.

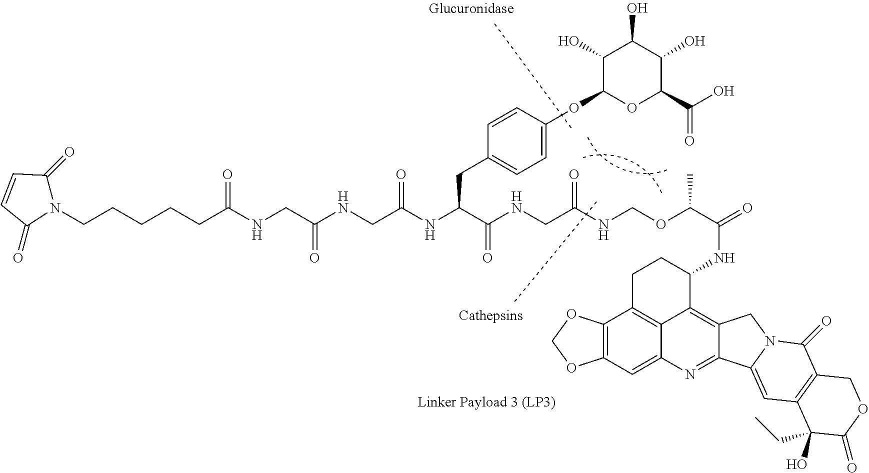 [SMILES: CC[C@@]1(O)C(=O)OCc2c1cc1n(c2=O)Cc2c-1nc1cc3c(c4c1c2[C@@H](NC(=O)[C@@H](C)OCNC(=O)CNC(=O)[C@H](Cc1ccc(O[C@@H]2O[C@H](C(=O)O)[C@@H](O)[C@H](O)[C@H]2O)cc1)NC(=O)CNC(=O)CNC(=O)CCCCCN1C(=O)C=CC1=O)CC4)OCO3]

The introduction of the β-glucuronide moiety may have the following advantages.

### Temporary Hydrophilic Unit:

(1) Increasing the overall aqueous solubility and conjugation efficiency thereby limiting the accumulation and aggregation of the ADC during conjugation as well as in circulation.

(2) Conjugation of challenging hydrophobic payloads, thus opening a window for expanding the scope of cytotoxic payloads in ADC.

### Sequential Enzymatic Cleavage:

(1) Cathepsin mediated peptide cleavage is hindered until cleavage of pendant group by the lysosomal β-glucuronidase.

(2) Limit the exposure of cleavage site in the linker-payload from the action of serum proteases and neutrophil elastases effectively reducing the premature drug loss in circulation and improve the preferential drug release in tumors.

### Conjugation:

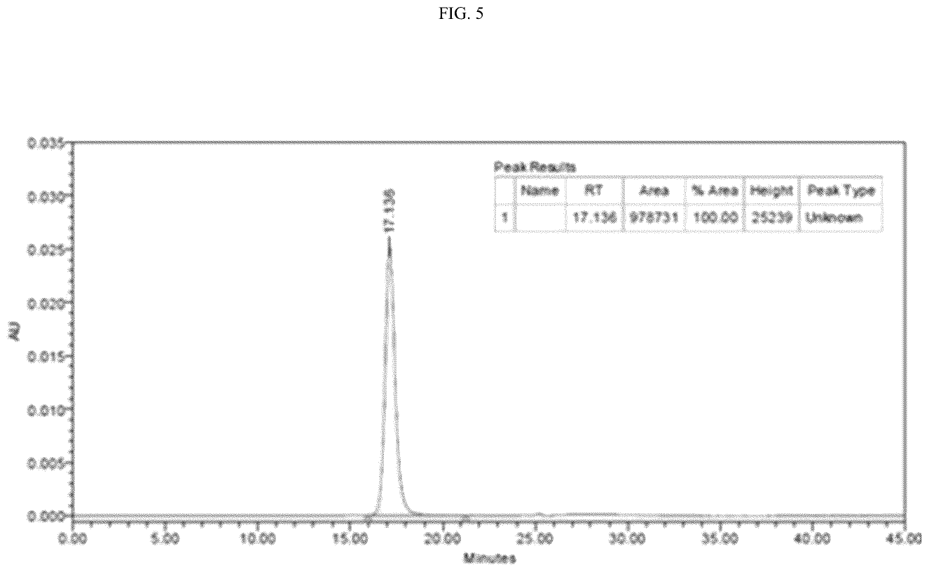

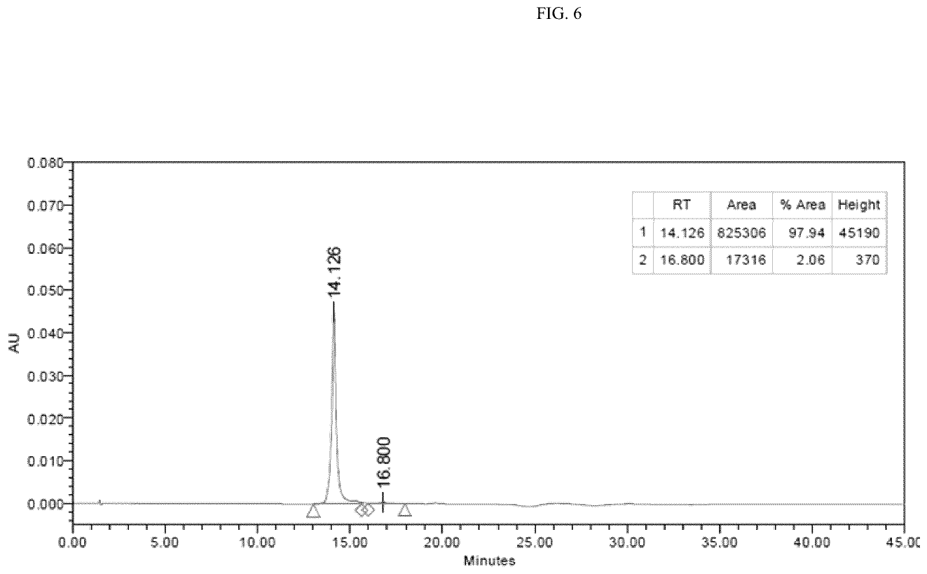

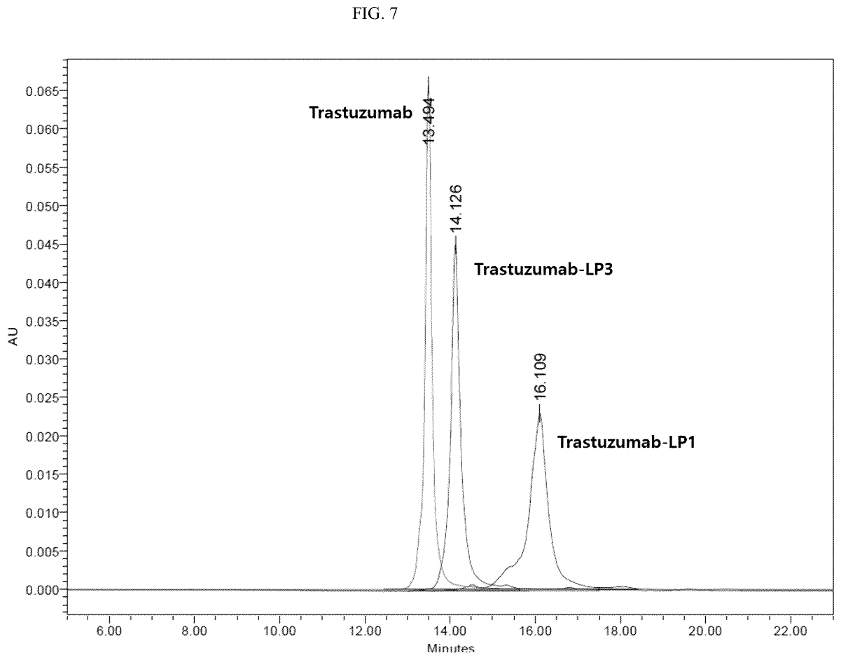

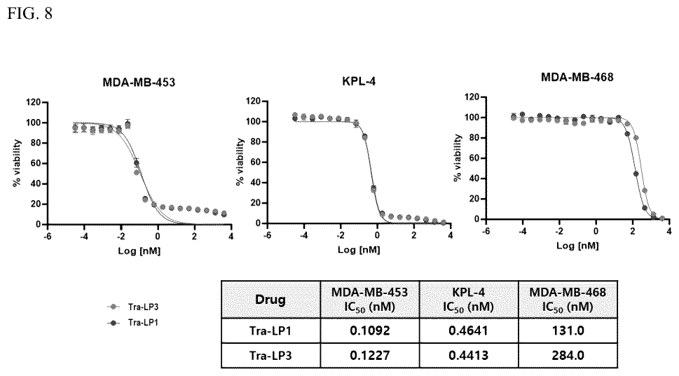

4 mg/ml of Trastuzumab was reacted with 30-fold molar excess of TCEP (Tris(2-carboxyethyl)phosphine hydrochloride) for 2 hrs at 25° C. to generate cysteine from disulfide bonds. Reduced Trastuzumab was purified from unreacted TCEP using PD-10 desalting column. 2 mg/ml of reduced Trastuzumab was reacted with 12-fold molar excess of linker-payload 3 (LP3) under 10% (v/v0) DMSO for 1 hour at 25° C. Trastuzumab ADC was purified from unreacted linker-payloads and DMSO using PD-10 desalting column. See FIGS. 5-8.

### (3) Improving Stability

Traditionally, maleimides have been extensively used for cysteine modification due to their rapid and selective reaction with thiols. However, recent findings indicate that the thioether linkage undergoes deconjugation through a retro-Michael pathway, resulting in premature drug release in the circulation and a decrease in therapeutic efficacy. Furthermore, maleimide-based conjugates undergo thiol exchange with other plasma thiols, such as human serum albumin (HSA), leading to off-site delivery of toxic payload and further compromising effectiveness.

To address these challenges and enhance plasma linker stability beyond maleimide-caproic acid-containing antibody-drug conjugates (ADCs), acetamide-linked conjugates were developed by substituting the maleimide moiety with bromoacetamide. These new linker derivatives (LP4 and LP5) featuring bromoacetamide react with thiols generated after disulfide bond reduction of the antibody. This results in the formation of stable conjugates and significantly reduce the premature drug release and off-target binding.

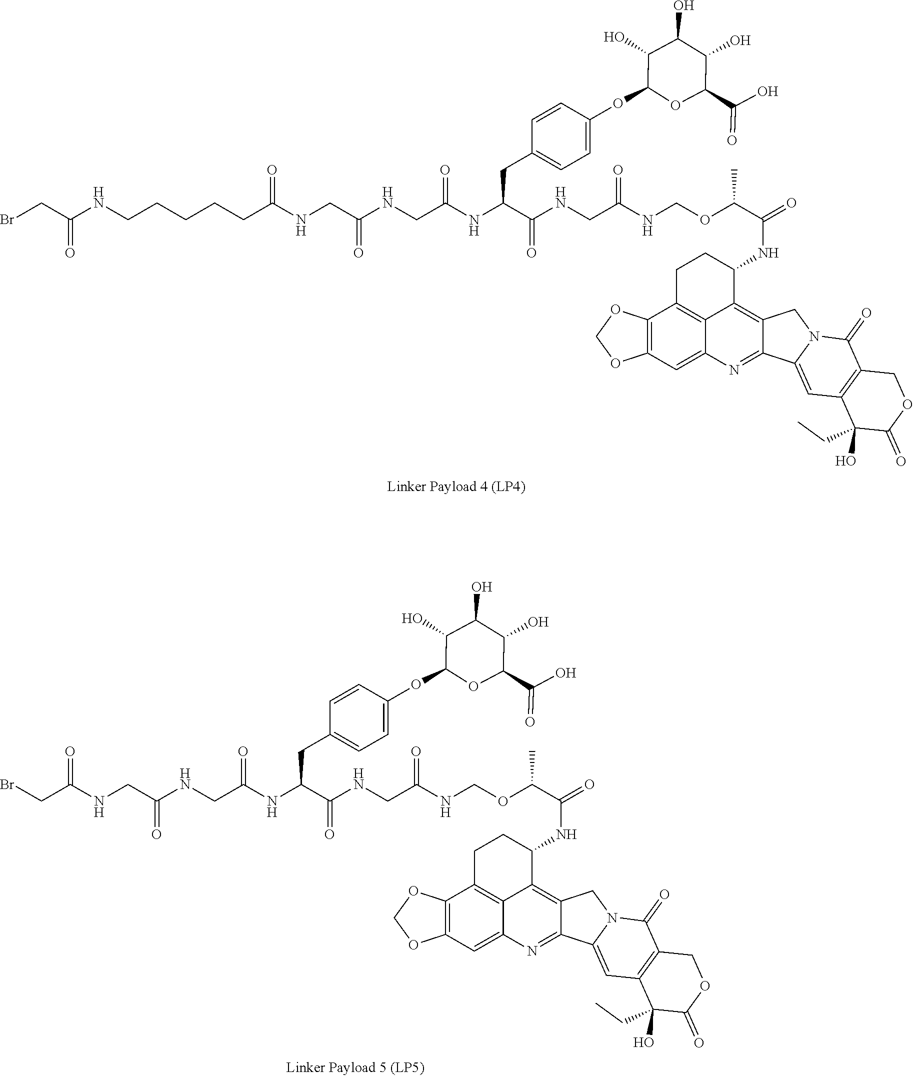 [SMILES: CC[C@@]1(O)C(=O)OCc2c1cc1n(c2=O)Cc2c-1nc1cc3c(c4c1c2[C@@H](NC(=O)[C@@H](C)OCNC(=O)CNC(=O)[C@H](Cc1ccc(O[C@@H]2O[C@H](C(=O)O)[C@@H](O)[C@H](O)[C@H]2O)cc1)NC(=O)CNC(=O)CNC(=O)CCCCCNC(=O)CBr)CC4)OCO3 ; CC[C@@]1(O)C(=O)OCc2c1cc1n(c2=O)Cc2c-1nc1cc3c(c4c1c2[C@@H](NC(=O)[C@@H](C)OCNC(=O)CNC(=O)[C@H](Cc1ccc(O[C@@H]2O[C@H](C(=O)O)[C@@H](O)[C@H](O)[C@H]2O)cc1)NC(=O)CNC(=O)CNC(=O)CBr)CC4)OCO3]

In addition to the acetamide-linked conjugates, we have developed a self-stabilizing maleimide conjugate. To achieve this, we incorporated amide functionality (LP6) into the caproic acid spacer, which facilitates the rapid hydrolysis of maleimide into its ring-open structure, enhancing the stability of the maleimide-drug conjugate. This modification effectively reduces retro-Michael deconjugation, ensuring a consistent DAR throughout the lifespan of the ADC in circulation. Alternatively, the maleimide conjugation site can be replaced with DBCO based conjugation to improve the stability (LP7).

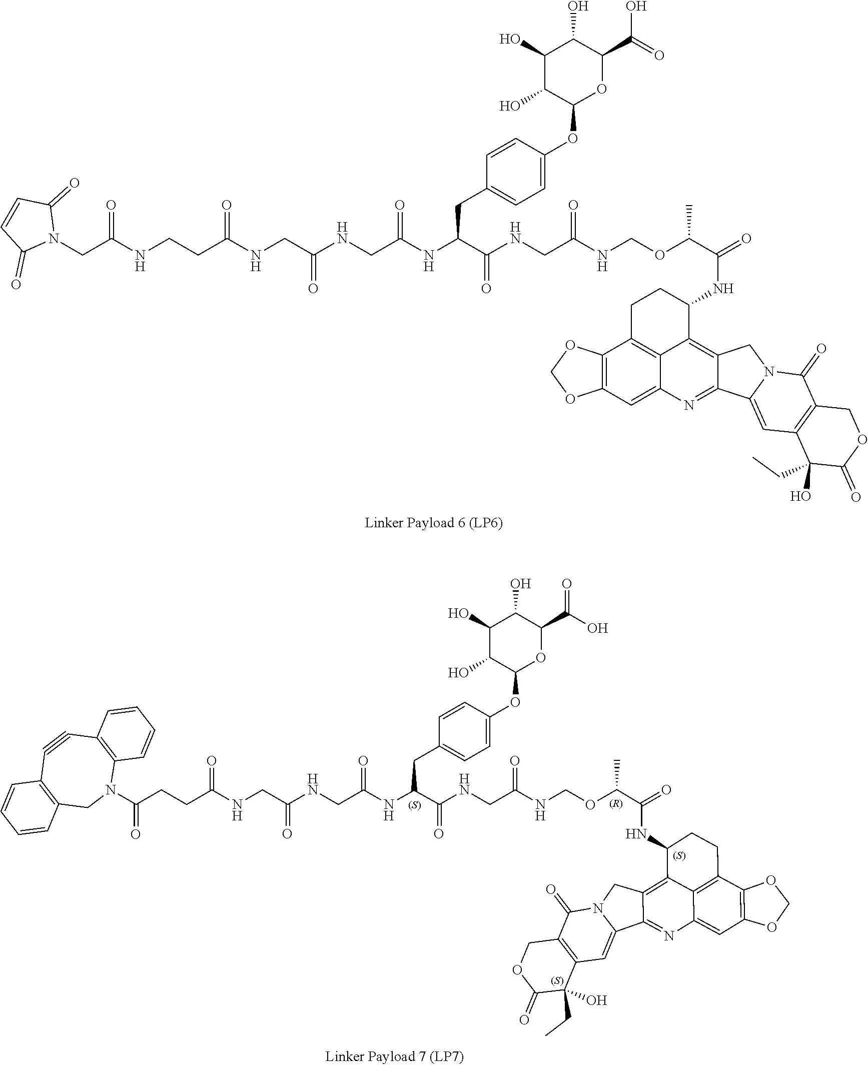

The inventors postulate that the present design will maintain the efficacy and increase the safety profile of the GGFG linker system. This will in turn lead to less off-target toxicities like neutropenia and interstitial lung disease.

Within this innovation, the objective of limiting the impact of the antitumor compound on normal cells is pursued by the integration of a hydrophilic pendant group into the linker's backbone. This enhances the overall hydrophilicity of linker-payload, rendering it challenging for the deconjugated linker-payload to permeate the cell wall. Consequently, the non-selective uptake of the antitumor compound is diminished, leading to a reduction in unintended cellular absorption and resulting in a lowered incidence of off-target effects and achieving a high level of safety, as reflected in the reduction of ILD.

### SYNTHETIC SCHEMES & PROCEDURES

### Synthesis of Linker-Payload 2 (LP2)

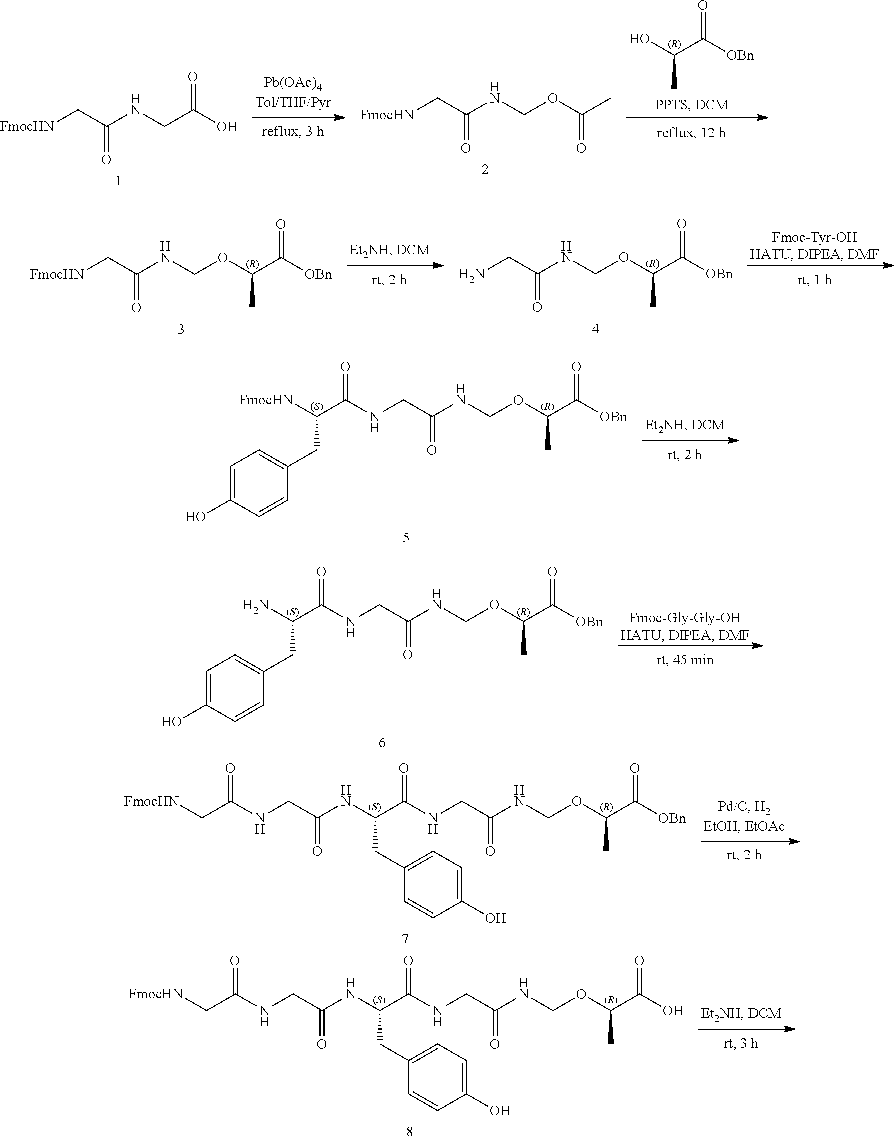

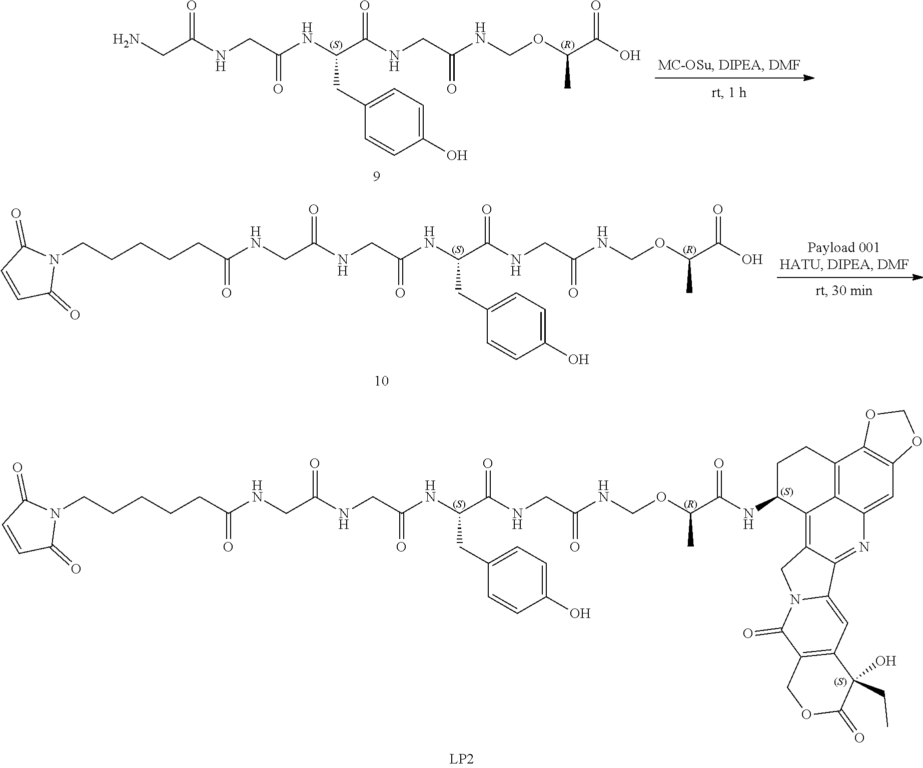

### Step 1:

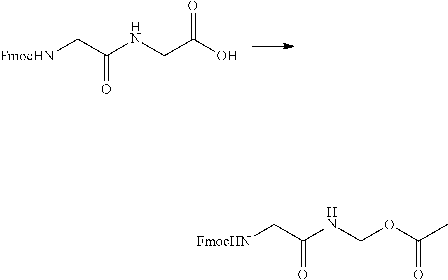

Fmoc-Gly-Gly-OH (5.0 g, 14.1 mmol) was partially dissolved in tetrahydrofuran (125 mL), toluene (42.6 mL) and pyridine (2.15 mL). Lead (IV) acetate (7.8 g, 17.6 mmol) was added, and the reaction mixture turned orange. The mixture was heated to reflux temperature. After stirring for 3 hours, the reaction mixture was cooled to room temperature then filtered through a bed of celite, rinsed with ethyl acetate and concentrated under reduced pressure. 10 The residue was purified by flash chromatography (silica, 10-100% ethyl acetate in heptane) to give (2-((((9H-fluoren-9-yl)methoxy)carbonyl)amino)acetamido)-methyl acetate (3.0 g, 57% yield) as a white solid. ¹H NMR (400 MHZ, CDCl₃) δ 7.77 (d, J=7.4 Hz, 2H), 7.59 (d, J=7.5 Hz, 2H), 7.45-7.37 (m, 2H), 7.36-7.29 (m, 2H), 6.98 (s, 1H), 5.34 (s, 1H), 5.26 (d, J=7.3 Hz, 2H), 4.46 (d, J=6.8 Hz, 2H), 4.23 (t, J=6.8 Hz, 1H), 3.94-3.84 (m, 2H), 2.06 (s, 3H). m/z 391.2 [M+Na]⁺

### Step 2:

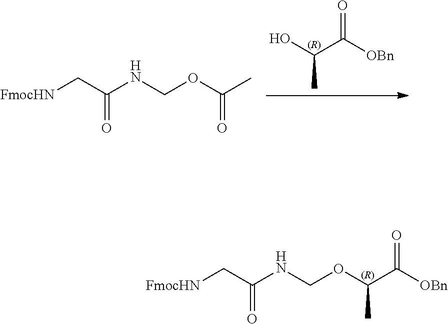

To a solution of (2-((((9H-fluoren-9-yl)methoxy)carbonyl)amino)acetamido)methyl acetate (1.27 g, 3.45 mmol) in dichloromethane (20 mL) was added benzyl (R)-Lactate (6.21 g, 34.5 mmol), followed by pyridinium p-toluenesulfonate (0.087 g, 0.345 mmol) and the mixture was stirred at reflux temperature overnight. The reaction mixture was cooled to room temperature, diluted with ethyl acetate (100 mL), washed with water (3×50 mL), dried over sodium sulfate, filtered, and concentrated to give 7.10 g colorless oil. The residue was purified by flash chromatography (80 g Si; 10% to 70% ethyl acetate in heptane). Product fractions were combined and concentrated to afford 1.68 g colorless turbid oil. ¹H NMR (400 MHz, CDCl₃) δ 7.77 (d, J=7.5 Hz, 2H), 7.58 (d, J=7.6 Hz, 2H), 7.45-7.28 (m, 8H), 6.83-6.71 (m, 1H), 5.30-5.21 (m, 1H), 5.21-5.10 (m, 2H), 4.91-4.73 (m, 2H), 4.45 (d, J=6.7 Hz, 2H), 4.30-4.17 (m, 2H), 3.86-3.69 (m, 2H), 1.41 (d, J=6.9 Hz, 3H). m/z 511.2 [M+H]⁺

### Step 3:

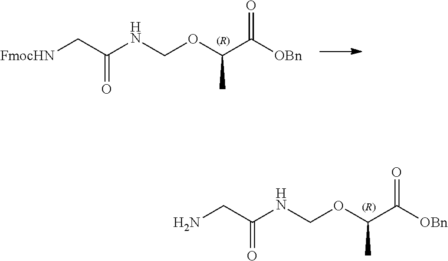

To a solution of benzyl (R)-1-(9H-fluoren-9-yl)-10-methyl-3,6-dioxo-2,9-dioxa-4,7-diazaundecan-11-oate (1.0 g, 1 eq, 2.0 mmol) was added diethylamine (7.5 g, 11 mL, 50 eq, 0.10 mol). The reaction mixture was stirred at room temperature for 1 hour. The reaction mixture was evaporated to dryness and co-evaporated twice with DCM. Yield: 926 mg; m/z 267.1 [M+H]⁺

### Step 4:

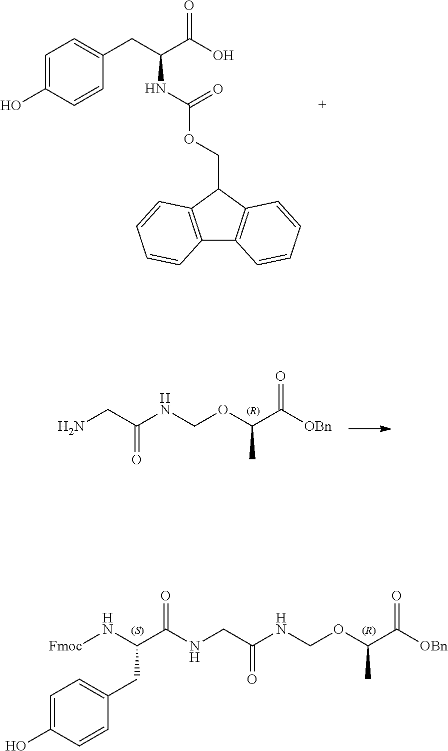

benzyl (R)-2-((2-aminoacetamido)methoxy)propanoate (0.71 g, estimated 58% Wt, 1 eq, 1.55 mmol) was dissolved in DMF (10.0 mL). (S)-2-((((9H-Fluoren-9-yl)methoxy)carbonyl)amino)-3-(4-hydroxy-phenyl)propanoic acid (624 mg, 1 eq, 1.55 mmol) and DIPEA (300 mg, 404 μL, 1.5 eq, 2.32 mmol) were added followed by HATU (588 mg, 1 eq, 1.55 mmol). The reaction mixture was stirred at room temperature for 30 minutes. The mixture was diluted with 100 mL water and extracted with 2×100 mL EtOAc.

The organic layer was dried over Na₂SO₄, filtered, and evaporated to dryness. The crude product was purified by flash column chromatography (24 g Si, 0-100% EtOAc in heptane). Evaporation of solvents afforded the product (455 mg, 45%) as a solid. ¹H NMR (400 MHZ, DMSO-d₆) δ 9.17 (s, 1H), 8.60 (t, J=6.8 Hz, 1H), 8.29 (t, J=5.8 Hz, 1H), 7.88 (d, J=7.5 Hz, 2H), 7.68-7.58 (m, 3H), 7.45-7.25 (m, 10H), 7.10-7.04 (m, 2H), 6.66-6.60 (m, 2H), 5.18-5.07 (m, 2H), 4.67-4.53 (m, 2H), 4.26-4.09 (m, 5H), 3.79-3.65 (m, 2H), 2.92 (dd, J=13.8, 4.1 Hz, 1H), 2.71-2.62 (m, 1H), 1.26 (d, J=6.8 Hz, 3H); m/z: 674.4 [M+Na]⁺

### Step 5:

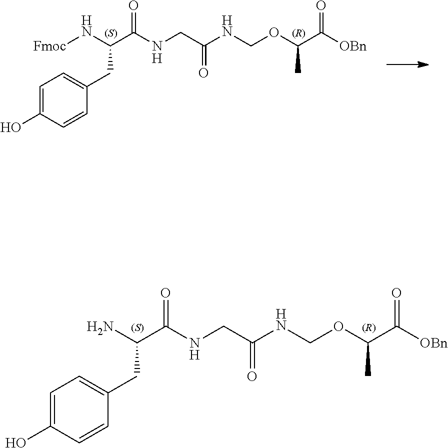

benzyl (5S,13R)-1-(9H-fluoren-9-yl)-5-(4-hydroxybenzyl)-13-methyl-3,6,9-trioxo-2,12-dioxa-4,7,10-tri-azatetradecan-14-oate (550 mg, 1 eq, 844 μmol) was suspended in DCM (4.3 mL). Diethylamine (3.09 g, 4.37 mL, 50 eq, 42.2 mmol) was added and the reaction mixture was stirred at rt for 3 hours. The reaction mixture was evaporated to dryness and co-evaporated twice with DCM to afford a turbid oil. Yield: 570 mg. m/z 430.2 [M+H]⁺

### Step 6:

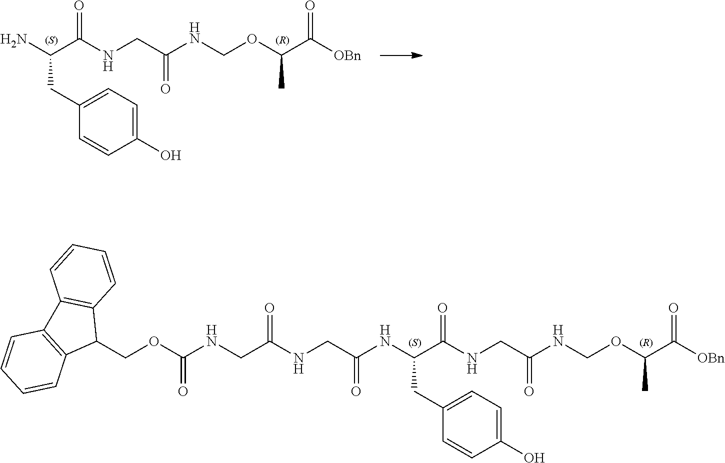

Crude benzyl (R)-2-((2-((S)-2-amino-3-(4-hydroxyphenyl)propanamido)acetamido) methoxy) propano-ate (570 mg, estimated 63% Wt, 1 eq, 836 μmol) was dissolved in dichloromethane (10 mL). 2-(2-((((9H-Fluoren-9-yl)methoxy)carbonyl)amino)acetamido)acetic acid (296 mg, 1.0 eq, 836 μmol) was added followed by HATU (318 mg, 1.0 eq, 836 μmol) and DIPEA (162 mg, 218 μL, 1.5 eq, 1.25 mmol). The yellow reaction mixture was stirred at room temperature. After 2 hours, 0.3 equiv. HATU and 0.5 equiv. DIPEA were added. The reaction mixture was stirred for a total of 5 hours. The mixture was evaporated to dryness and purified by flash column chromatography (24 g Si, 0-6% MeOH in DCM). Product fractions were evaporated to dryness which gave benzyl (11S,19R)-1-(9H-fluoren-9-yl)-11-(4-hydroxybenzyl)-19-methyl-3,6,9,12,15-pentaoxo-2,18-dioxa-4,7,10,13,16-pentaazaicosan-20-oate (190 mg, 248 μmol, 29%); ¹H NMR (400 MHz, DMSO-d₆) δ 9.16 (s, 1H), 8.54 (t, J=6.8 Hz, 1H), 8.26 (t, J=5.9 Hz, 1H), 8.09-7.98 (m, 2H), 7.89 (d, J=7.5 Hz, 2H), 7.70 (d, J=7.4 Hz, 2H), 7.59 (t, J=6.1 Hz, 1H), 7.45-7.28 (m, 9H), 7.04-6.97 (m, 2H), 6.66-6.60 (m, 2H), 5.20-5.09 (m, 2H), 4.66-4.55 (m, 2H), 4.46-4.37 (m, 1H), 4.32-4.17 (m, 4H), 3.82-3.53 (m, 6H), 3.19-3.07 (m, 1H), 2.93 (dd, J=14.0, 4.6 Hz, 1H), 2.68 (dd, J=14.0, 9.3 Hz, 1H), 1.31-1.21 (m, 6H); m/z 788.4 [M+Na]⁺

### Step 7:

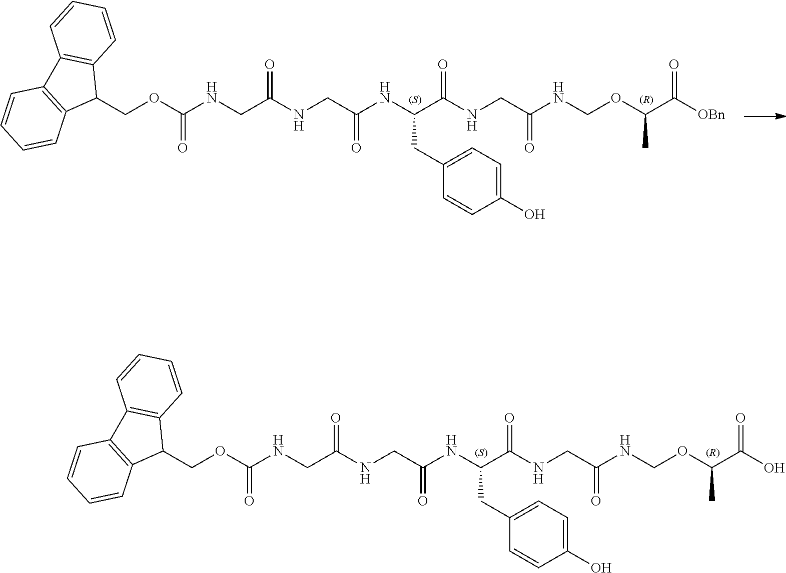

benzyl (11S,19R)-1-(9H-fluoren-9-yl)-11-(4-hydroxybenzyl)-19-methyl-3,6,9,12,15-pentaoxo-2,18-dioxa-4,7,10,13, 16-pentaazaicosan-20-oate (175 mg, 1 eq, 229 μmol) was dissolved in ethanol (5.0 mL)/ethyl acetate (5.0 mL). Pd/C (10%, 50% wet) (48 mg, 5% Wt, 0.1 eq, 22.9 μmol) was added and the reaction mixture was stirred under a hydrogen atmosphere for 2.5 hours. The reaction mixture was filtered over celite and washed with 2×20 mL methanol. The filtrate was evaporated to dryness affording a colorless solid (169 mg). m/z 674.4 [M−H]⁻

### Step 8:

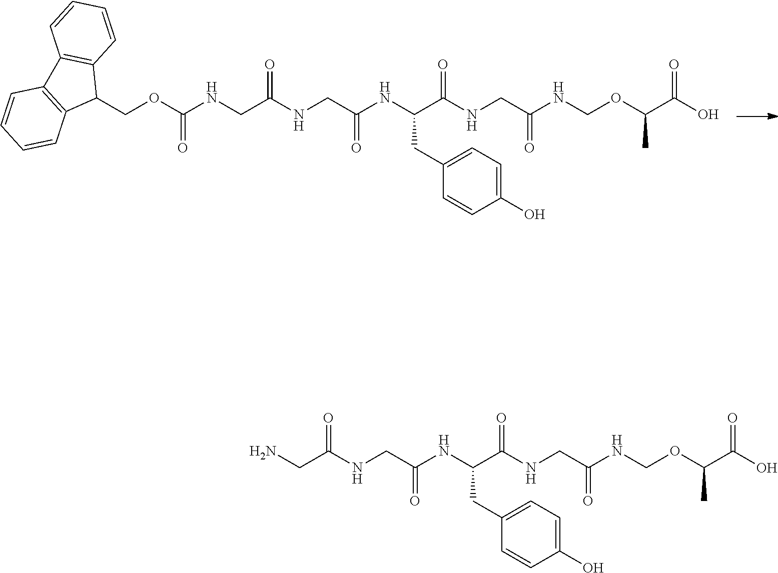 [SMILES: C[C@@H](OCNC(=O)CNC(=O)[C@H](Cc1ccc(O)cc1)NC(=O)CNC(=O)CNC(=O)OCC1c2ccccc2-c2ccccc21)C(=O)O ; C[C@@H](OCNC(=O)CNC(=O)[C@H](Cc1ccc(O)cc1)NC(=O)CNC(=O)CN)C(=O)O]

(11S,19R)-1-(9H-fluoren-9-yl)-11-(4-hydroxybenzyl)-19-methyl-3,6,9,12,15-pentaoxo-2,18-dioxa-4,7,10, 13,16-pentaazaicosan-20-oic acid (154 mg, 1 eq, 228 μmol) (crude) was suspended in 1 mL DCM. Diethylamine (833 mg, 1.18 mL, 50 eq, 11.4 mmol) was added and the reaction mixture was stirred at room temperature for 2 hours. A solid formed on the bottom of the flask. The DCM layer was removed. The solid was dried under reduced pressure to afford 154 mg of a white solid which was used directly in the next reaction without further purification. m/z 454.2 [M+H]⁺

### Step 9:

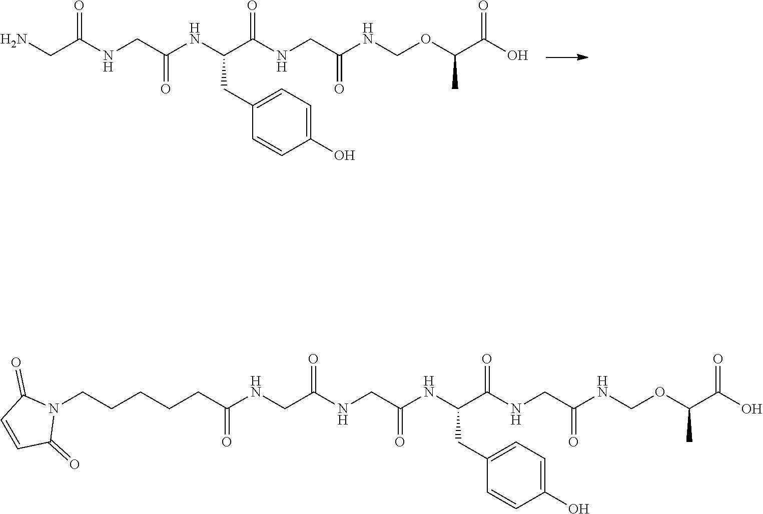 [SMILES: C[C@@H](OCNC(=O)CNC(=O)[C@H](Cc1ccc(O)cc1)NC(=O)CNC(=O)CN)C(=O)O ; C[C@@H](OCNC(=O)CNC(=O)[C@H](Cc1ccc(O)cc1)NC(=O)CNC(=O)CNC(=O)CCCCCN1C(=O)C=CC1=O)C(=O)O]

(2R,10S)-16-amino-10-(4-hydroxybenzyl)-2-methyl-6,9,12,15-tetraoxo-3-oxa-5,8,11,14-tetraaza hexa-decanoic acid (154 mg, 67% Wt, 1 eq, 228 μmol) (crude) was suspended in 1.5 mL DMF. DIPEA (88.2 mg, 119 μL, 3 eq, 683 μmol) was added. (2,5-dioxopyrrolidin-1-yl) 6-(2,5-dioxopyrrol-1-yl)hexanoate (105 mg, 1.5 eq, 341 μmol) was added. The reaction was stirred at room temperature for 20 minutes. The reaction mixture was diluted with DCM and added to a short column of silica gel (˜30g). The column was first eluted with 100 mL 9/1 DCM/MeOH. Then, the product was eluted with 75 mL 1/1 DCM/MeOH. Product containing fractions were evaporated to dryness under reduced pressure. The crude product was triturated from DCM to afford a white solid. The product batch was taken up in DMSO and purified by preparative MPLC (Luna5-40). Product fractions were lyophilized which gave the product as a white solid. Yield: 80 mg, 54%. ¹H NMR (400 MHZ, DMSO-d₆) δ 12.56 (s, 1H), 9.16 (s, 1H), 8.51 (t, J=6.7 Hz, 1H), 8.23 (t, J=5.9 Hz, 1H), 8.12-7.99 (m, 3H), 7.04-6.97 (m, 4H), 6.66-6.59 (m, 2H), 4.65-4.52 (m, 2H), 4.44-4.35 (m, 1H), 4.08-3.99 (m, 1H), 3.78-3.56 (m, 6H), 3.37 (t, J=7.1 Hz, 2H), 2.92 (dd, J=13.9, 4.8 Hz, 1H), 2.68 (dd, J=14.0, 9.5 Hz, 1H), 2.15-2.06 (m, 2H), 1.54-1.41 (m, 4H), 1.27-1.13 (m, 5H); m/z 645.4 [M−H]⁻, 669.4 [M+Na]⁺

### Step 10:

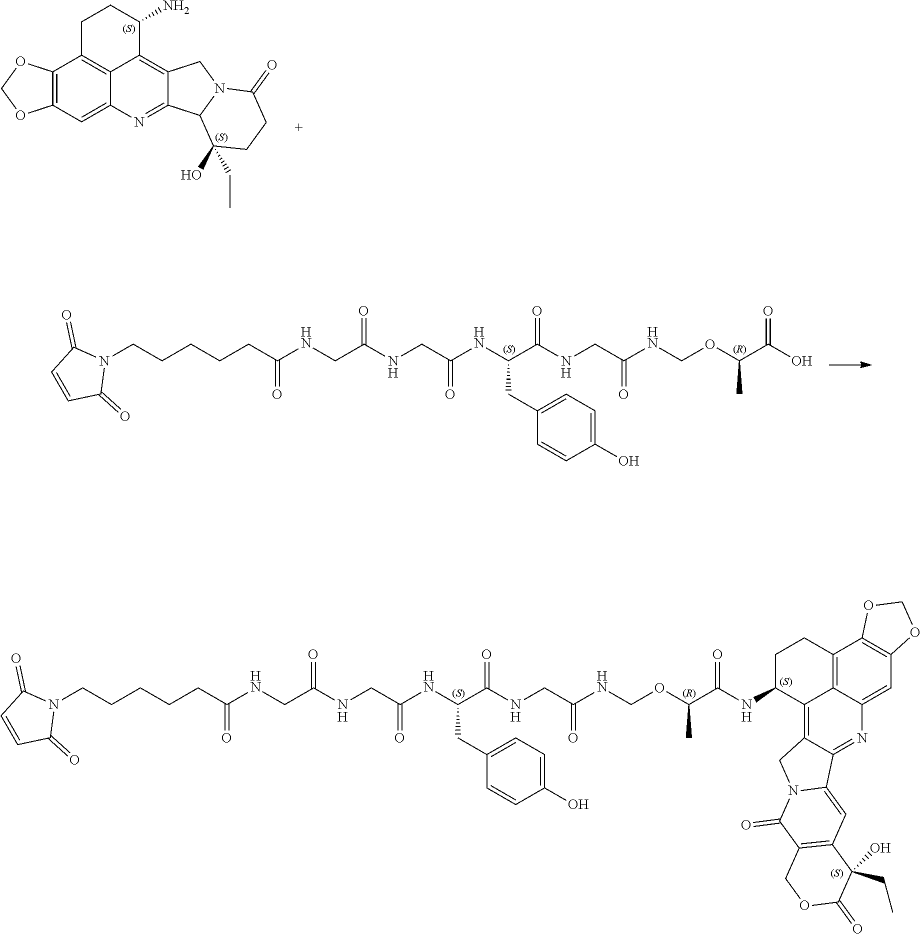

(1S,10S)-1-amino-10-ethyl-10-hydroxy-1,2,3,10,13,16-hexahydro-11H,14H-benzo[de][1,3]dioxolo [4,5-g]pyrano[3′,4′:6,7]indolizino[1,2-b]quinoline-11,14-dione (20 mg, 1 eq, 45 μmol) was suspended in DMF (2 mL). DIPEA (35 mg, 47 μL, 6 eq, 0.27 mmol) was added. (2R, 10S)-23-(2,5-dioxo-2,5-dihydro-1H-pyrrol-1-yl)-10-(4-hydroxybenzyl)-2-methyl-6,9,12,15,18-pentaoxo-3-oxa-5,8,11, 14, 17-pentaazatricosa-noic acid (26 mg, 0.9 eq, 40 μmol) was added followed by HATU (34 mg, 2 eq, 89 μmol). The reaction mixture was stirred at room temperature for 1 hour. The reaction mixture was purified directly by acidic preparative MPLC. Product fractions were combined and lyophilized which gave a yellow solid. Yield: 22 mg, 51%. ¹H NMR (400 MHZ, DMSO-d₆) δ 9.14 (s, 1H), 8.57 (t, J=6.7 Hz, 1H), 8.49 (d, J=9.2 Hz, 1H), 8.24 (t, J=5.9 Hz, 1H), 8.08-7.96 (m, 3H), 7.40 (s, 1H), 7.23 (s, 1H), 7.01-6.94 (m, 4H), 6.64-6.58 (m, 2H), 6.47 (s, 1H), 6.27 (d, J=5.8 Hz, 2H), 5.60-5.52 (m, 1H), 5.44-5.34 (m, 2H), 5.17-5.03 (m, 2H), 4.67 (dd, J=10.1, 6.6 Hz, 1H), 4.53 (dd, J=10.2, 6.6 Hz, 1H), 4.40-4.32 (m, 1H), 4.15-4.06 (m, 1H), 3.77-3.54 (m, 6H), 3.39-3.33 (m, 2H), 3.15-2.97 (m, 2H), 2.87 (dd, J=13.9, 4.7 Hz, 1H), 2.67-2.59 (m, 1H), 2.17-2.05 (m, 4H), 1.92-1.77 (m, 2H), 1.51-1.41 (m, 4H), 1.39 (d, J=6.8 Hz, 3H), 1.23-1.13 (m, 2H), 0.87 (t, J=7.3 Hz, 3H). m/z 1076.4 [M+H]⁺

### Synthesis of Linker Payload 3 (LP3)

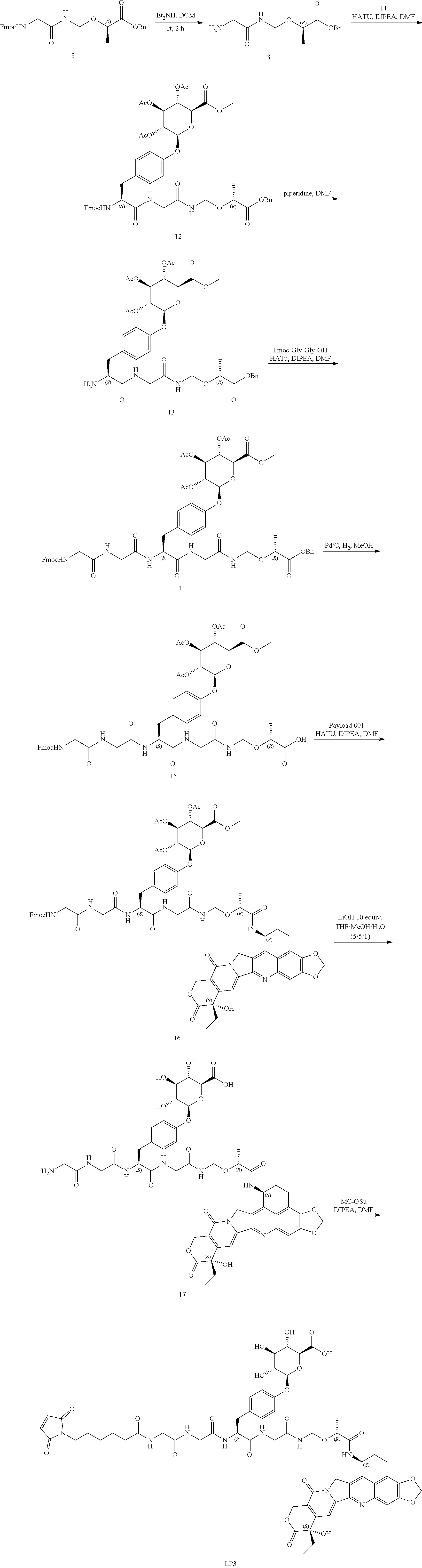

### Step 1:

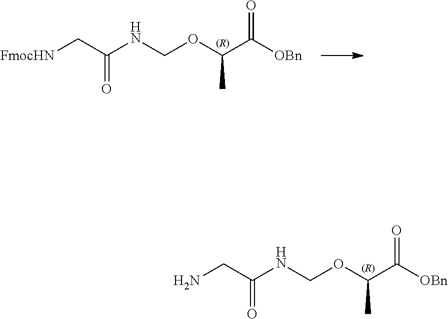

To a suspension of benzyl (R)-1-(9H-fluoren-9-yl)-10-methyl-3.6-dioxo-2.9-dioxa-4.7-diazaundecan-11-oate (211 mg, 1 eq, 432 μmol) in Dichloromethane (2.0 mL) was added diethylamine (0.71 g, 1.0 mL, 22 eq, 9.7 mmol). The reaction mixture was stirred for 90 minutes at rt. After 90 minutes, the reaction mixture was concentrated under reduced pressure and co-evaporated with dichloromethane (6×) to afford the product a thick colorless oil. m/z 267.2 [M+H]⁺

### Step 2:

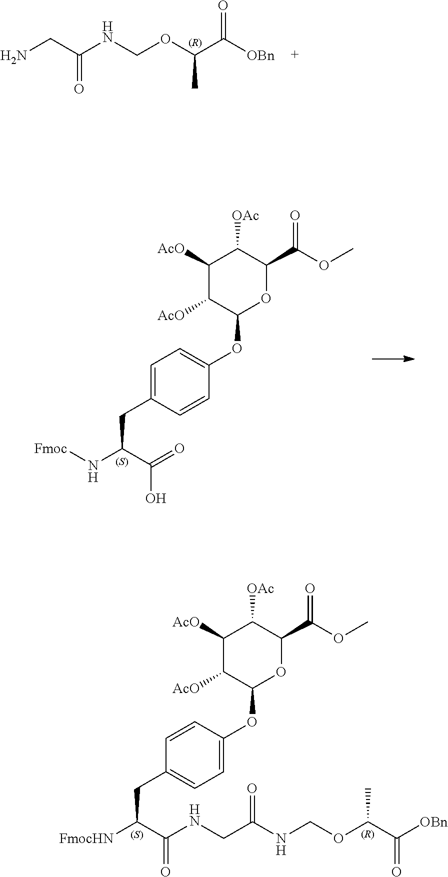

To a solution of benzyl (R)-2-((2-aminoacetamido)methoxy)propanoate (115 mg, 1 eq, 432 μmol) in DMF (5.0 mL) was added DIPEA (167 mg, 226 μL, 3 eq, 1.30 mmol) followed by (S)-2-((((9H-fluoren-9-yl)methoxy)carbonyl)amino)-3-(4-(((2S,3R,4S,5S,6S)-3,4,5-triacetoxy-6-(methoxy-carbonyl)tetrahydro-2H-pyran-2-yl)oxy)phenyl)propanoic acid (466 mg, 1.5 eq, 648 μmol) and HATU (246 mg, 1.5 eq, 648 μmol). The reaction mixture was stirred at rt for 1 hour. The reaction mixture was diluted with ethyl acetate and washed with water (2×) and brine (2×). The organic layer was dried with Na₂SO₄, filtered, and concentrated to afford a thick yellow oil (656 mg). The crude product was purified via flash column chromatography (24 g Si, 0-100% EtOAc in heptane). Product fractions were collected, concentrated and co-evaporated to afford the product as a white solid (317 mg, 71%). ¹H NMR (400 MHZ, CDCl₃) δ 7.76 (d, J=7.5 Hz, 2H), 7.56-7.48 (m, 2H), 7.40 (t, J=7.5 Hz, 2H), 7.37-7.27 (m, 7H), 7.14-7.05 (m, 2H), 6.94 (d, J=8.1 Hz, 2H), 6.87 (s, 1H), 6.29 (s, 1H), 5.36-5.19 (m, 4H), 5.18-5.05 (m, 3H), 4.86-4.78 (m, 1H), 4.72-4.64 (m, 1H), 4.53-4.44 (m, 1H), 4.43-4.26 (m, 2H), 4.24-4.08 (m, 2H), 3.79 (m, 2H), 3.70 (s, 3H), 3.15-2.91 (m, 2H), 2.08-2.00 (m, 9H), 1.61-1.53 (m, 2H), 1.38 (d, J=7.0 Hz, 3H). m/z 990.2 [M+Na]⁺

### Step 3:

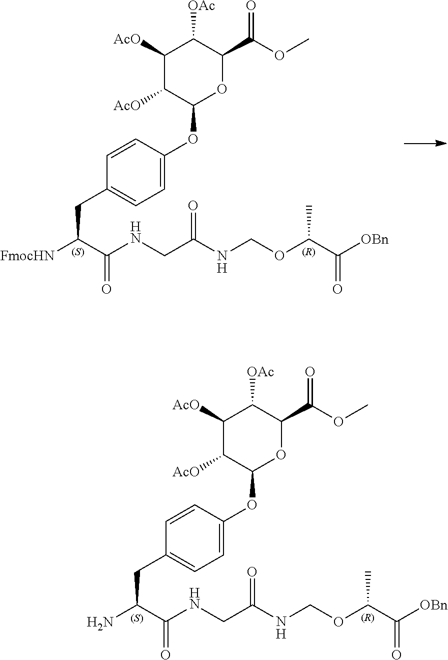

To a solution of (2S,3R,4S,5S,6S)-2-(4-((4R,12S)-12-((((9H-fluoren-9-yl)methoxy)carbonyl)amino)-4-methyl-3,8,11-trioxo-1-phenyl-2,5-dioxa-7,10-diazatridecan-13-yl)phenoxy)-6-(methoxy-carbonyl)tetra-hydro-2H-pyran-3,4,5-triyl triacetate (317 mg, 1 eq, 327 μmol) in DMF (4.5 mL) was added piperidine (61.3 mg, 71.2 μL, 2.2 eq, 720 μmol) and the reaction mixture was stirred at room temperature for 30 minutes. The reaction mixture was quenched with acetic acid (43.3 mg, 41.2 μL, 2.2 eq, 720 μmol) and purified by acidic preparative MPLC (Luna10-50). Product fractions were combined and lyophilized to afford a white solid (200 mg, 82%). ¹H NMR (400 MHZ, CDCl₃) δ 7.81 (t, J=5.8 Hz, 1H), 7.38-7.30 (m, 5H), 7.17-7.11 (m, 2H), 6.99-6.92 (m, 2H), 6.83 (t, J=6.8 Hz, 1H), 5.39-5.25 (m, 3H), 5.18 (d, J=4.7 Hz, 2H), 5.16-5.12 (m, 1H), 4.88-4.73 (m, 2H), 4.29-4.14 (m, 2H), 3.89-3.85 (m, 2H), 3.73 (s, 3H), 3.63 (dd, J=8.9, 4.2 Hz, 1H), 3.17 (dd, J=13.7, 4.2 Hz, 1H), 2.80-2.70 (m, 1H), 2.10-2.01 (m, 9H), 1.42 (d, J=6.9 Hz, 3H). m/z 746.4 [M+H]⁺

### Step 4:

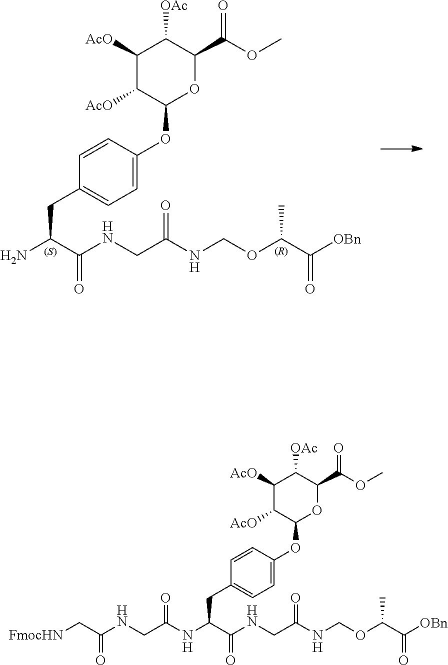

To a solution of (2S,3R,4S,5S,6S)-2-(4-((4R,12S)-12-amino-4-methyl-3,8,11-trioxo-1-phenyl-2,5-dioxa-7,10-diazatridecan-13-yl)phenoxy)-6-(methoxycarbonyl)tetrahydro-2H-pyran-3,4,5-triyl triacetate (197 mg, 1 eq, 264 μmol) in DMF (3.0 mL) was added DIPEA (102 mg, 138 μL, 3 eq, 793 μmol) followed by Fmoc-Gly-Gly-OH (140 mg, 1.5 eq, 396 μmol) and HATU (151 mg, 1.5 eq, 396 μmol). The reaction mixture was stirred for 45 minutes at rt. The reaction mixture was purified directly by acidic preparative MPLC (Luna30-70). Product fractions were combined and lyophilized to afford a white solid (215 mg, 75%). ¹H NMR (400 MHZ, CDCl₃) δ 7.75 (d, J=7.6 Hz, 2H), 7.57 (t, J=6.7 Hz, 2H), 7.49-7.27 (m, 10H), 7.20-7.04 (m, 5H), 6.92-6.85 (m, 2H), 5.97 (t, J=5.6 Hz, 1H), 5.39-5.27 (m, 2H), 5.27-5.20 (m, 1H), 5.18-5.06 (m, 3H), 4.80-4.72 (m, 1H), 4.71-4.63 (m, 1H), 4.56 (q, J=7.1 Hz, 1H), 4.44 (d, J=6.7 Hz, 2H), 4.27-4.15 (m, 3H), 3.94-3.72 (m, 6H), 3.68 (s, 3H), 3.21-3.12 (m, 1H), 2.99-2.89 (m, 1H), 2.08-2.01 (m, 9H), 1.36 (d, J=6.9 Hz, 3H). m/z 1104.2 [M+Na]⁺

### Step 5:

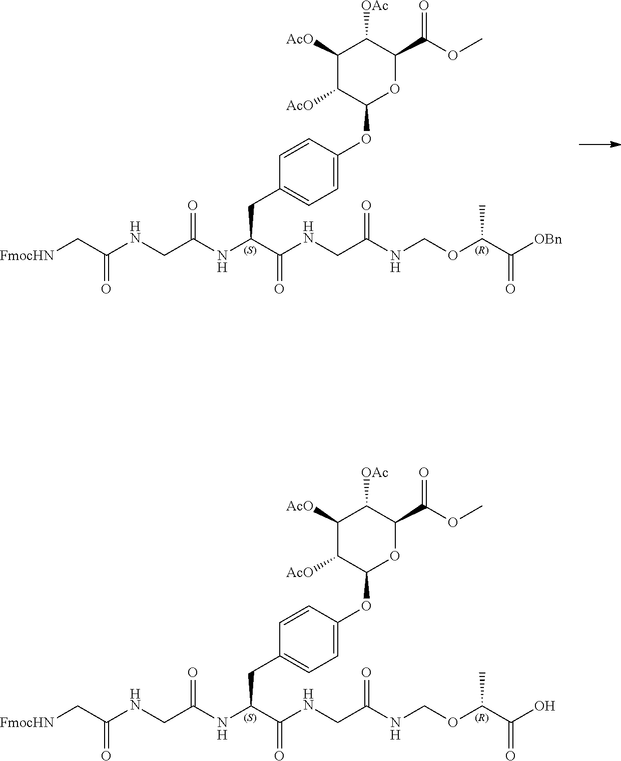

A suspension of (2S,3R,4S,5S,6S)-2-(4-((4R,12S)-12-(2-(2-((((9H-fluoren-9-yl)methoxy)carbonyl)amino)-acetamido)acetamido)-4-methyl-3,8,11-trioxo-1-phenyl-2,5-dioxa-7,10-diazatridecan-13-yl)phenoxy)-6-(methoxycarbonyl) tetrahydro-2H-pyran-3,4,5-triyl triacetate (193 mg, 1 eq, 178 μmol) in methanol (8.0 mL) was purged with nitrogen for 10 min. Then, Pd/C (10%, 50% wet) (38.0 mg, 5% Wt, 0.1 eq, 17.8 μmol) was added and the mixture was stirred under a hydrogen atmosphere for 30 minutes. The reaction mixture was flushed with nitrogen for 10 minutes, filtered over Kieselguhr, eluted with methanol, and concentrated to give a white solid (155 mg). The crude product was purified by acidic preparative MPLC (Luna20-60). Product fractions were combined and lyophilized to afford a white solid (117 mg, 66%). ¹H NMR (400 MHZ, DMSO) δ 12.60 (s, 1H), 8.64-8.57 (m, 1H), 8.31 (t, J=5.8 Hz, 1H), 8.22-8.04 (m, 2H), 7.89 (d, J=7.5 Hz, 2H), 7.71 (d, J=7.4 Hz, 2H), 7.61 (t, J=6.1 Hz, 1H), 7.41 (t, J=7.4 Hz, 2H), 7.32 (t, J=7.4 Hz, 2H), 7.22-7.15 (m, 2H), 6.91-6.85 (m, 2H), 5.61 (d, J=8.0 Hz, 1H), 5.45 (t, J=9.6 Hz, 1H), 5.11-5.01 (m, 2H), 4.72-4.52 (m, 3H), 4.52-4.42 (m, 1H), 4.32-4.26 (m, 2H), 4.26-4.15 (m, 1H), 4.03 (q, J=6.9 Hz, 1H), 3.83-3.55 (m, 9H), 3.04-2.95 (m, 1H), 2.81-2.71 (m, 1H), 2.04-1.96 (m, 9H), 1.23 (d, J=6.9 Hz, 3H). m/z 1014.0 [M+Na]⁺

### Step 6:

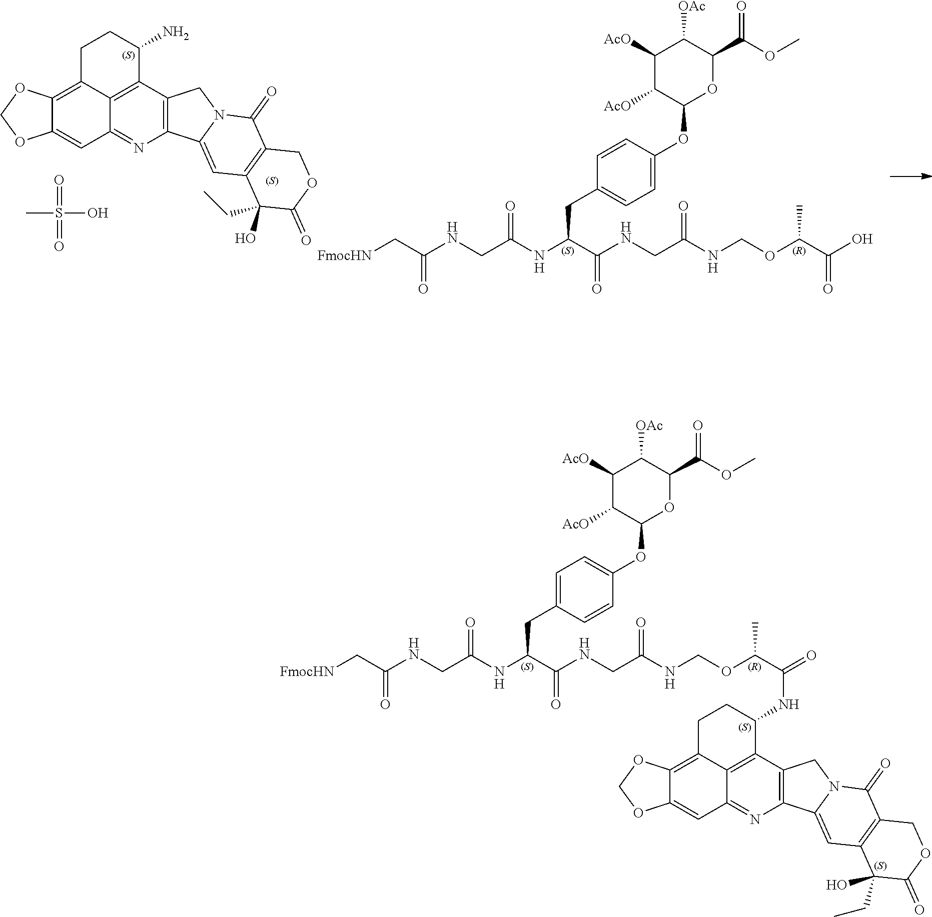

(1S, 10S)-1-amino-10-ethyl-10-hydroxy-1,2,3,10,13,16-hexahydro-11H,14H-benzo[de][1,3]dioxolo [4,5-g]pyrano [3′,4′:6,7]indolizino[1,2-b]quinoline-11,14-dione methanesulfonate (55 mg, 1 eq, 0.10 mmol) was suspended in DMF (2.5 mL). (11S,19R)-1-(9H-fluoren-9-yl)-19-methyl-3,6,9,12,15-pentaoxo-11-(4-(((2S,3R,4S,5S,6S)-3,4,5-triacetoxy-6-(methoxycarbonyl)tetrahydro-2H-pyran-2-yl)oxy)benzyl)-2,18-dioxa-4,7,10,13,16-pentaazaicosan-20-oic acid (0.10 g, 1 eq, 0.10 mmol) and DIPEA (39 mg, 53 μL, 3 eq, 0.30 mmol) were added followed by HATU (48 mg, 1.25 eq, 0.13 mmol). The reaction mixture was stirred at room temperature for 15 minutes. The reaction mixture was purified directly by acidic preparative MPLC (Luna20-60). Product fractions were combined and lyophilized to afford a yellow solid (100 mg, 70%). ¹H NMR (400 MHZ, DMSO-d₆) δ 8.65 (t, J=6.6 Hz, 1H), 8.49 (d, J=9.1 Hz, 1H), 8.31 (t, J=5.8 Hz, 1H), 8.10 (d, J=7.9 Hz, 1H), 8.03 (t, J=5.7 Hz, 1H), 7.87 (d, J=7.6 Hz, 2H), 7.69 (d, J=7.4 Hz, 2H), 7.57 (t, J=6.0 Hz, 1H), 7.43-7.26 (m, 5H), 7.23 (s, 1H), 7.19-7.11 (m, 2H), 6.91-6.84 (m, 2H), 6.47 (s, 1H), 6.26 (d, J=7.2 Hz, 2H), 5.64-5.51 (m, 2H), 5.45 (t, J=9.7 Hz, 1H), 5.42-5.32 (m, 2H), 5.17-4.99 (m, 4H), 4.72-4.63 (m, 2H), 4.54 (m, 1H), 4.48-4.38 (m, 1H), 4.31-4.07 (m, 4H), 3.81-3.55 (m, 9H), 3.15-2.88 (m, 3H), 2.75-2.67 (m, 1H), 2.16-2.05 (m, 2H), 2.05-1.95 (m, 9H), 1.92-1.77 (m, 2H), 1.39 (d, J=6.8 Hz, 3H), 0.86 (t, J=7.3 Hz, 3H). m/z 1421.8 [M+H]⁺

### Step 7:

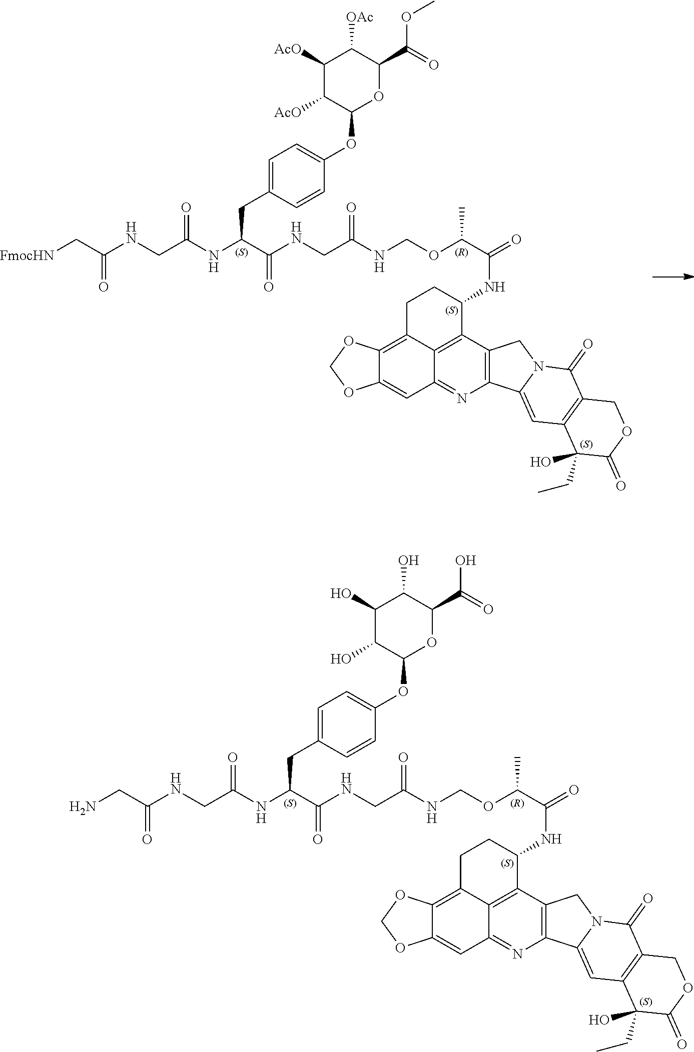

(2S,3R,4S,5S,6S)-2-(4-((S)-11-((2-(((((R)-1-(((1S,10S)-10-ethyl-10-hydroxy-11,14-dioxo-2,3,10,11, 14,16-hexahydro-1H,13H-benzo[de][1,3]dioxolo[4,5-g]pyrano[3′,4′:6, 7]indolizino[1,2-b]quinolin-1-yl)amino)-1-oxopropan-2-yl)oxy)methyl)amino)-2-oxoethyl)carbamoyl)-1-(9H-fluoren-9-yl)-3,6,9-trioxo-2-oxa-4,7,10-triazadodecan-12-yl)phenoxy)-6-(methoxycarbonyl)tetra hydro-2H-pyran-3,4,5-triyl triacetate (50 mg, 1 eq, 35 μmol) was dissolved in MeOH/THF (1:1, 6 mL) and cooled to 0° C. Lithium hydroxide monohydrate (15 mg, 10 eq, 0.35 mmol) in 600 μL H₂O was added. The reaction mixture was stirred at 0° C. for 2 hours. Acetic acid (0.11 g, 0.10 mL, 50 eq, 1.8 mmol) was added and the organic solvents were removed under reduced pressure. To the aqueous residue was added DMSO (2 mL). The solution was purified by acidic preparative MPLC (Luna5-40). Product fractions were combined and lyophilized to afford the product as a white solid (27 mg, 72%). ¹H NMR (400 MHZ, DMSO-d₆) δ 8.71 (t, J=6.6 Hz, 1H), 8.54 (d, J=9.2 Hz, 1H), 8.38 (t, J=5.9 Hz, 1H), 8.29-8.13 (m, 1H), 7.93 (d, J=8.2 Hz, 1H), 7.40 (s, 1H), 7.23 (s, 1H), 7.08 (d, J=8.3 Hz, 2H), 6.86 (d, J=8.2 Hz, 2H), 6.48 (s, 1H), 6.26 (d, J=5.0 Hz, 2H), 5.61-5.51 (m, 1H), 5.45-5.34 (m, 2H), 5.29-4.98 (m, 4H), 4.84 (d, J=7.5 Hz, 1H), 4.74-4.64 (m, 1H), 4.58-4.41 (m, 2H), 4.12 (q, J=6.7 Hz, 1H), 3.82-3.48 (m, 9H, coincides with H₂O peak), 3.24-2.92 (m, 13H, coincides with H₂O peak), 2.64-2.53 (m, 1H), 2.17-2.08 (m, 2H), 1.92-1.78 (m, 2H), 1.39 (d, J=6.8 Hz, 3H), 0.87 (t, J=7.4 Hz, 3H). m/z 1059.6 [M+H]⁺

### Step 8:

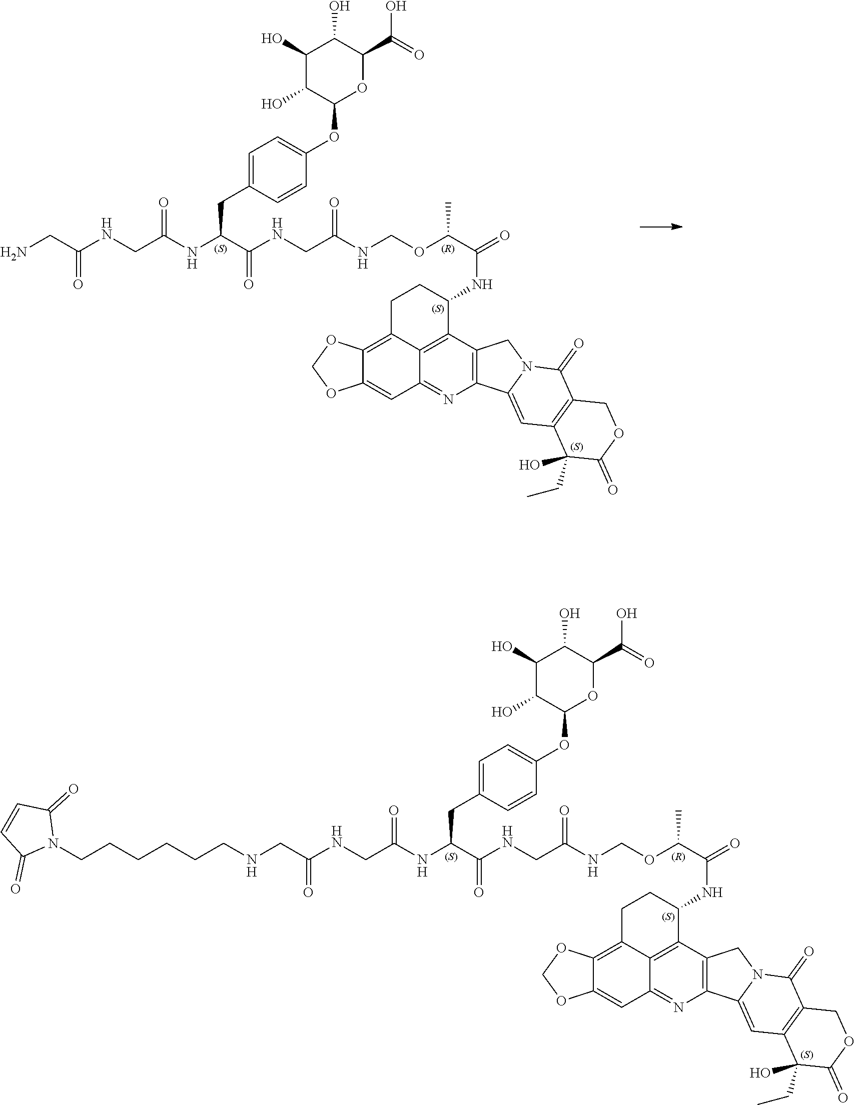

(2S,3S,4S,5R,6S)-6-(4-((S)-2-(2-(2-aminoacetamido)acetamido)-3-((2-(((((R)-1-(((1S,10S)-10-ethyl-10-hydroxy-11,14-dioxo-2,3,10,11,14,16-hexahydro-1H,13H-benzo[de][1,3]dioxolo[4,5-g]pyrano [3′,4′:6,7]-indolizino[1,2-b]quinolin-1-yl)amino)-1-oxopropan-2-yl)oxy)methyl)amino)-2-oxoethyl)amino)-3-oxo-propyl)phenoxy)-3,4,5-trihydroxytetrahydro-2H-pyran-2-carboxylic acid (60 mg, 1 eq, 57 μmol) and N-succinimidyl 6-maleimidohexanoate (35 mg, 2 eq, 0.11 mmol) were dissolved in DMF (2.5 mL). DIPEA (15 mg, 20 μL, 2 eq, 0.11 mmol) was added. The reaction mixture was stirred at room temperature for 45 minutes. The reaction mixture was purified directly by acidic preparative MPLC (Luna5-50). Product fractions were combined and lyophilized to afford the product as a light-yellow solid (49 mg, 69%). ¹H NMR (400 MHZ, DMSO-d₆) δ 8.62 (t, J=6.6 Hz, 1H), 8.50 (d, J=9.1 Hz, 1H), 8.27 (t, J=5.9 Hz, 1H), 8.11-7.98 (m, 3H), 7.39 (s, 1H), 7.23 (s, 1H), 7.15-7.07 (m, 2H), 6.98 (s, 2H), 6.92-6.84 (m, 2H), 6.47 (s, 1H), 6.26 (d, J=4.3 Hz, 2H), 5.61-5.52 (m, 1H), 5.46-5.32 (m, 3H), 5.24-4.93 (m, 4H), 4.67 (dd, J=10.1, 6.6 Hz, 1H), 4.53 (dd, J=10.1, 6.6 Hz, 1H), 4.46-4.36 (m, 1H), 4.11 (q, J=6.7 Hz, 1H), 3.86 (d, J=9.5 Hz, 1H), 3.80-3.53 (m, 7H), 3.43-3.36 (m, 2H), 3.29-3.19 (m, 2H), 3.16-2.89 (m, 3H), 2.69 (dd, J=13.9, 9.1 Hz, 1H), 2.19-2.03 (m, 4H), 1.94-1.76 (m, 2H), 1.52-1.37 (m, 7H), 1.23-1.11 (m, 2H), 0.87 (t, J=7.3 Hz, 3H). m/z 1252.2 [M+H]⁺

### Synthesis of Compound 11

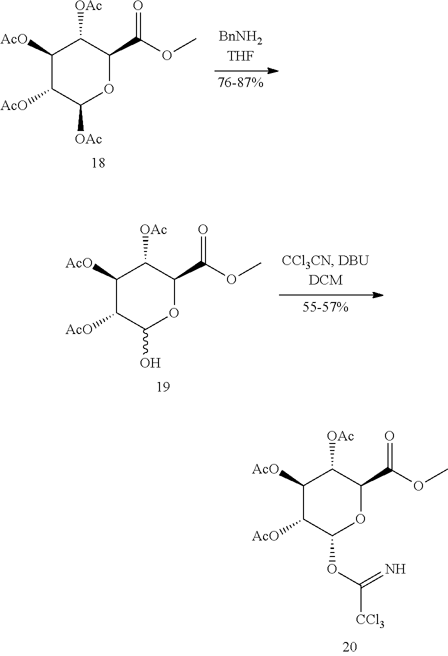

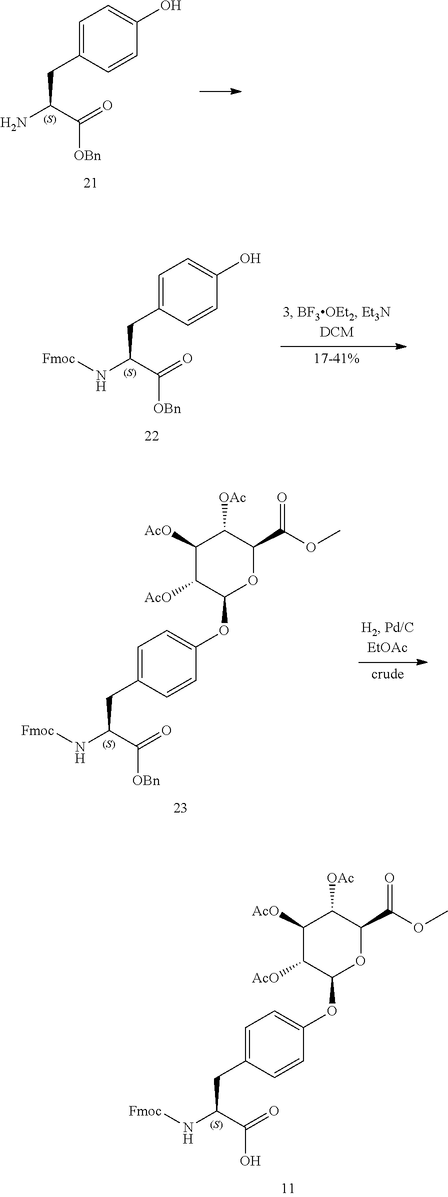

### Step 1:

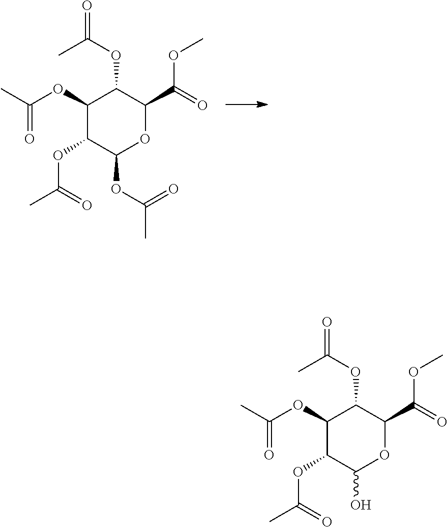 [SMILES: COC(=O)[C@H]1O[C@@H](OC(C)=O)[C@H](OC(C)=O)[C@@H](OC(C)=O)[C@@H]1OC(C)=O ; COC(=O)[C@H]1OC(O)[C@H](OC(C)=O)[C@@H](OC(C)=O)[C@@H]1OC(C)=O]

Benzylamine (1.16 g, 1.18 mL, 1.2 eq, 10.8 mmol) was added to a suspension of Methyl 1,2,3,4-tetra-O-acetyl-beta-D-glucuronate (3.39 g, 1 eq, 9.01 mmol) in THF (40 mL). The reaction mixture was stirred overnight at rt. The reaction mixture was concentrated in vacuo to afford a red oil. The crude material was purified by flash column chromatography over silica (80 g Si, 0-50% EtOAc in Heptane) to afford a red oil (3.12 g). The material was coated onto isolute and purified by flash column chromatography over silica (80 g Si, 0-50% EtOAc in Heptane) to afford the product as a yellowish oil (2.35g, 76%). ¹H NMR (400 MHz, CDCl₃) mixture of anomers: 8 5.62-5.53 (m, 2H), 5.35-5.15 (m, 1.5H), 4.96-4.89 (m, 1.25H), 4.83-4.78 (m, 0.25H), 4.59 (d, J=10.0 Hz, 1H), 4.17-4.07 (m, 1H), 3.79-3.72 (m, 4H), 3.72-3.53 (m, 1H), 2.13-2.07 (m, 4H), 2.07-1.99 (m, 8H). m/z 691.0 [2M+Na]⁺

### Step 2:

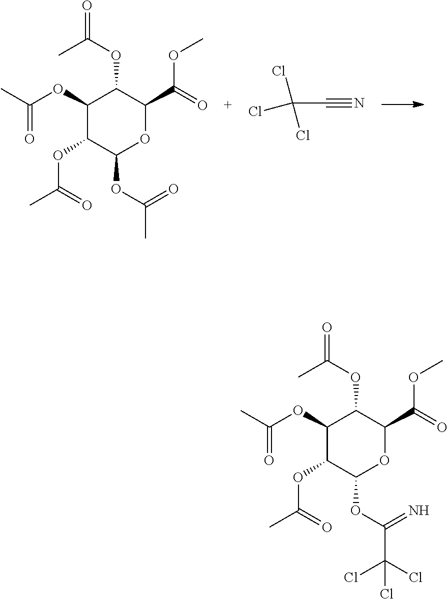 [SMILES: COC(=O)[C@H]1O[C@@H](OC(C)=O)[C@H](OC(C)=O)[C@@H](OC(C)=O)[C@@H]1OC(C)=O ; N#CC(Cl)(Cl)Cl ; COC(=O)[C@H]1O[C@H](OC(=N)C(Cl)(Cl)Cl)[C@H](OC(C)=O)[C@@H](OC(C)=O)[C@@H]1OC(C)=O]

A solution of (3R,4S,5S,6S)-2-hydroxy-6-(methoxycarbonyl)tetrahydro-2H-pyran-3,4,5-triyl triacetate (2.35 g, 1 eq, 7.03 mmol) and DCM (40 mL) was cooled to 0° C., whereafter 2,2,2-trichloroacetonitrile (5.07 g, 3.52 mL, 5 eq, 35.2 mmol) and DBU (214 mg, 210 μL, 0.2 eq, 1.41 mmol) were added subsequently. The reaction mixture was allowed to warm to room temperature and stirred for 3 hours. The reaction mixture was concentrated under reduced pressure to afford a red oil. The crude product was coated onto isolute and purified by flash column chromatography (80 g Si, 0-35% EtOAc in Heptane). Product fractions were collected and concentrated under reduced pressure to afford the product as a beige solid (1.86 g, 55%). ¹H NMR (400 MHZ, CDCl₃) δ 8.74 (s, 1H), 6.64 (d, J=3.6 Hz, 1H), 5.63 (t, J=9.9 Hz, 1H), 5.31-5.23 (m, 1H), 5.15 (dd, J=10.2, 3.6 Hz, 1H), 4.50 (d, J=10.2 Hz, 1H), 3.75 (s, 3H), 2.07-2.04 (m, 6H), 2.02 (s, 3H). m/z 499.8 [M+Na]⁺

### Step 3:

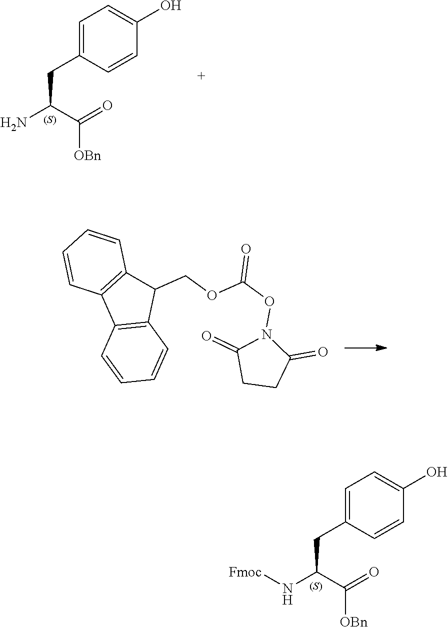

To a solution of H-Tyr-OBzl (4.0 g, 1 eq, 15 mmol) in chloroform (100 mL) was added Fmoc-OSu (5.0 g, 1 eq, 15 mmol) and the reaction mixture was stirred overnight at rt. The reaction mixture was diluted with DCM (100 mL) and washed with H₂O (2×100 mL) and brine (100 mL). The organic layer was concentrated under reduced pressure to afford an off-white solid (7.22 g). The solids were placed on a filter and rinsed with DCM (3×10 mL). The solids were taken and dried in a vacuum oven at 40° C.for 2 days to afford the product as a white solid (5.77 g, 79%). ¹H NMR (400 MHZ, DMSO) δ 9.25 (s, 1H), 7.95-7.81 (m, 3H), 7.66 (t, J=7.4 Hz, 1H), 7.53-7.20 (m, 9H), 7.08-6.89 (m, 2H), 6.70-6.60 (m, 2H), 5.15-5.01 (m, 2H), 4.48-3.97 (m, 4H), 3.02-2.61 (m, 2H). m/z 494.4 [M+H]⁺, 516.4 [M+Na]⁺

### Step 4:

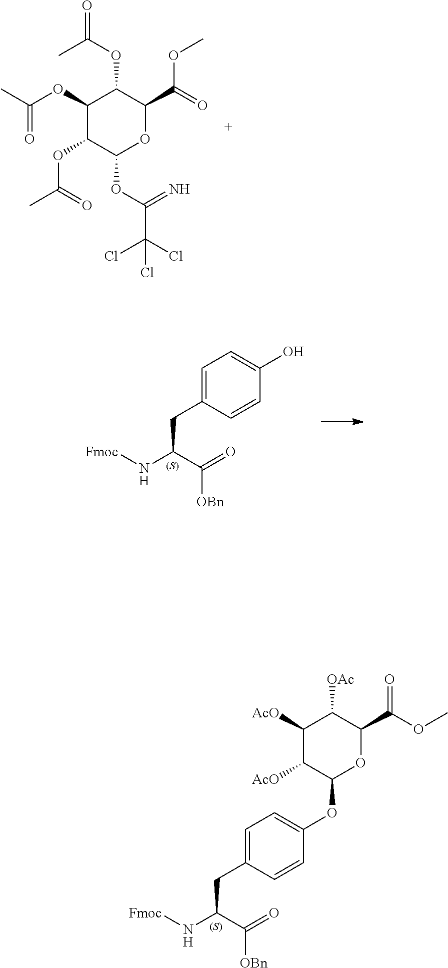

A suspension of (2S,3S,4S,5R)-2-(methoxycarbonyl)-6-(2,2,2-trichloro-1-iminoethoxy)tetrahydro-2H-pyran-3,4,5-triyl triacetate (51 mg, 1 eq, 0.11 mmol) and benzyl (((9H-fluoren-9-yl)methoxy)carbonyl)-L-tyrosinate (53 mg, 1 eq, 0.11 mmol) in DCM (dry) (1.5 mL) containing 4A molecular sieves was cooled to 0° C. BF₃·OEt₂ (ca. 48% BF3) (17 mg, 15 μL, 1.1 eq, 0.12 mmol) was added. The ice-bath was removed, and the reaction mixture was stirred at rt for 2 hours. The reaction mixture was quenched with Trimethylamine (2M in THF) (7.0 mg, 59 μL, 2.0 molar, 1.1 eq, 0.12 mmol) and purified directly by flash column chromatography (12 g Si, 0-60% EtOAc in Heptane). Product fractions were collected and concentrated under reduced pressure to afford the product as a white solid (32 mg, 37%). ¹H NMR (400 MHZ, CDCl₃) δ 7.78 (d, J=7.6 Hz, 2H), 7.59-7.51 (m, 2H), 7.45-7.28 (m, 9H), 6.93-6.75 (m, 4H), 5.37-5.09 (m, 6H), 5.02 (d, J=7.1 Hz, 1H), 4.73-4.65 (m, 1H), 4.49-4.42 (m, 1H), 4.37-4.29 (m, 1H), 4.23-4.18 (m, 1H), 4.16-4.06 (m, 1H), 3.71 (s, 3H), 3.15-2.99 (m, 2H), 2.09-2.02 (m, 9H). m/z 810.6 [M+H]⁺, 832.6 [M+Na]⁺

### Step 5:

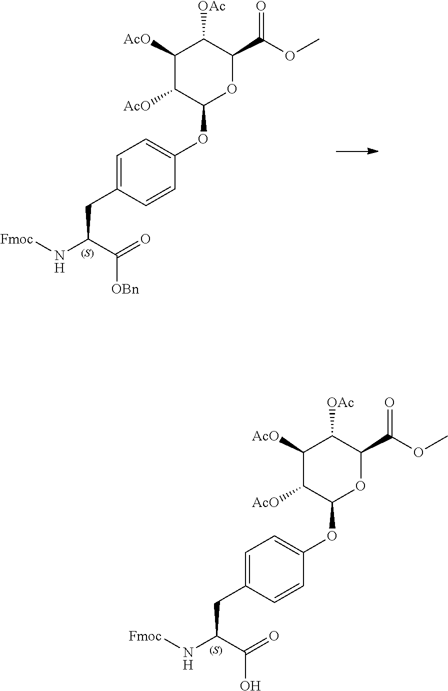

A suspension of (2S,3R,4S,5S,6S)-2-(4-((S)-2-((((9H-fluoren-9-yl)methoxy)carbonyl)amino)-3-(benzyloxy)-3-oxopropyl)phenoxy)-6-(methoxycarbonyl)tetrahydro-2H-pyran-3,4,5-triyl triacetate (527 mg, 1 eq, 651 μmol) in EtOAc (17 mL) was purged with nitrogen for 10 min. Then, Pd/C (10%, 50% wet) (139 mg, 5% Wt, 0.1 eq, 65.1 μmol) was added and the mixture was stirred under a hydrogen atmosphere for 5 h. The reaction mixture was flushed with nitrogen and then filtered through bed of Kieselguhr, rinsed with ethyl acetate and concentrated under reduced pressure, co-evaporated with dichloromethane (2×) to afford the product as a white solid (468 mg, quantitative yield). ¹H NMR (400 MHZ, CDCl3) δ 7.77 (d, J=7.6 Hz, 2H), 7.59-7.49 (m, 2H), 7.41 (t, J=7.5 Hz, 2H), 7.35-7.28 (m, 2H), 7.07 (d, J=8.1 Hz, 2H), 6.91 (d, J=8.0 Hz, 2H), 5.36-5.29 (m, 2H), 5.29-5.18 (m, 2H), 5.06 (d, J=7.3 Hz, 1H), 4.70-4.61 (m, 1H), 4.52-4.43 (m, 1H), 4.39-4.30 (m, 1H), 4.23-4.06 (m, 2H), 3.68 (s, 3H), 3.21-3.11 (m, 1H), 3.11-3.02 (m, 1H), 2.09-1.98 (m, 9H). m/z 742.0 [M+Na]⁺

### Synthesis of Linker Payload 4 (LP4)

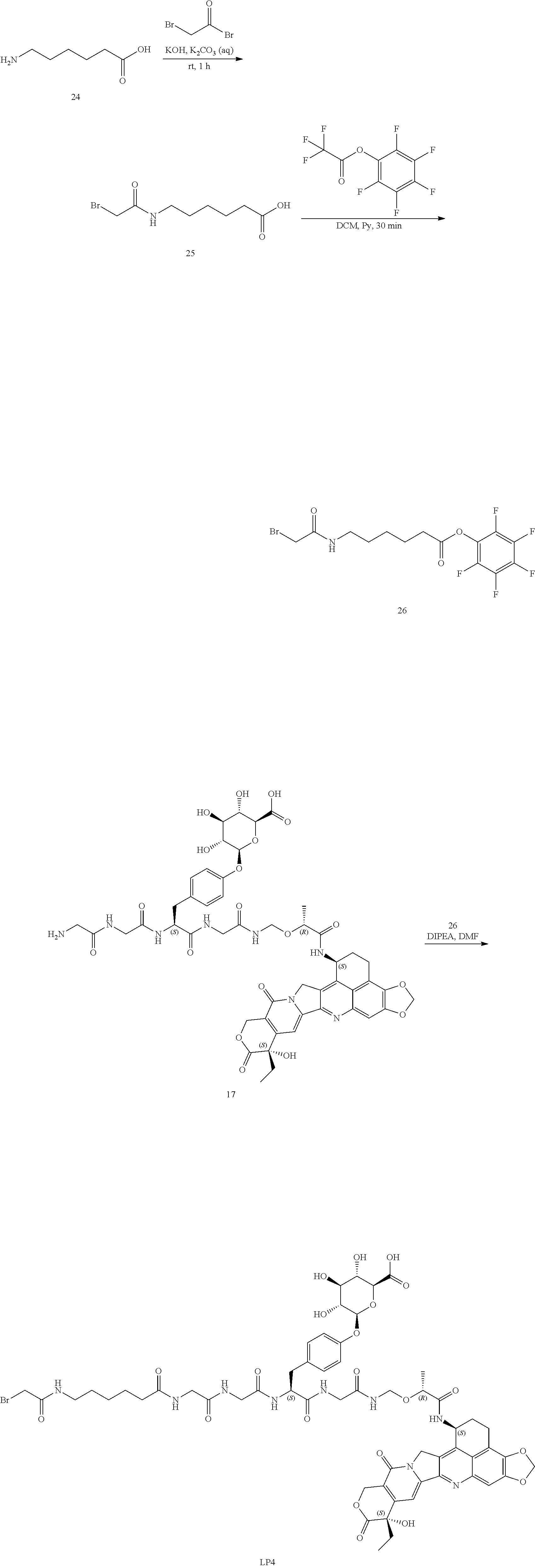

### Step 1:

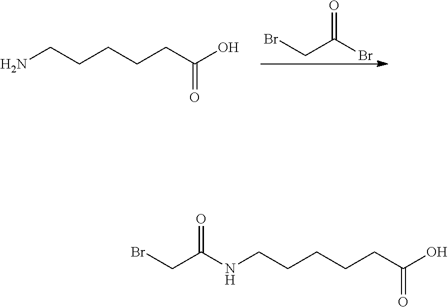 [SMILES: NCCCCCC(=O)O ; O=C(Br)CBr ; O=C(O)CCCCCNC(=O)CBr]

To a solution of potassium hydroxide (0.43 g, 1 eq, 7.6 mmol) in water (2.1 mL) at 0° C. was added 6-aminohexanoic acid (1.0 g, 1 eq, 7.6 mmol). Bromoacetyl bromide (1.8 g, 0.80 mL, 1.2 eq, 9.1 mmol) was added dropwise while an aqueous 2.8 M potassium carbonate solution was added dropwise to adjust the pH>7.8. After the addition, the reaction mixture was stirred at 0° C.for 1 h. The reaction was acidified with 0.5 N HCl to PH˜1 and extracted with ethyl acetate (3×). The combined organic layers were dried over sodium sulfate, filtered, and concentrated to give 1.47 g of a colorless oil. Purification by flash column chromatography (40 g Si; 0-2.5% methanol in dichloromethane). Product fractions were combined and concentrated to afford 661 mg (34%) of a colorless oil which solidified upon standing at room temperature. ¹H NMR (400 MHZ, CDCl₃) δ 6.52 (s, 1H), 3.89 (s, 2H), 3.35-3.26 (m, 2H), 2.38 (t, J=7.3 Hz, 2H), 1.73-1.63 (m, 2H), 1.63-1.52 (m, 2H), 1.46-1.34 (m, 2H). m/z 525.2 [M+H]⁺, Br isotope pattern

### Step 2:

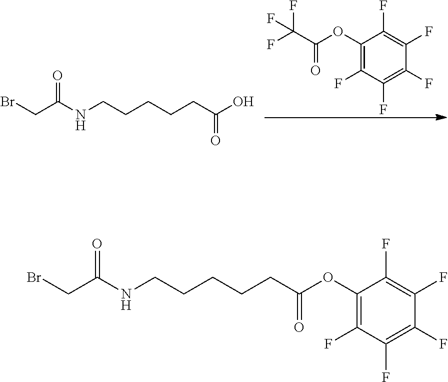 [SMILES: O=C(O)CCCCCNC(=O)CBr ; O=C(Oc1c(F)c(F)c(F)c(F)c1F)C(F)(F)F ; O=C(CBr)NCCCCCC(=O)Oc1c(F)c(F)c(F)c(F)c1F]

To a solution of 6-(2-bromoacetamido)hexanoic acid (631 mg, 1 eq, 2.50 mmol) in dichloromethane (30 mL) was added pentafluorophenyl trifluoroacetate (1.02 g, 624 μL, 1.45 eq, 3.63 mmol) and pyridine (792 mg, 810 μL, 4 eq, 10.0 mmol) at 0° C. The reaction mixture was stirred at 0° C.for 10 minutes. The reaction mixture was washed with aqueous 0.5 M HCl, and the organic layer was dried over sodium sulfate, filtered, and concentrated to give 1.2 g of a colorless oil. Purification by flash column chromatography (40 g Si; 0-50% ethyl acetate in heptane). Product fractions were combined and concentrated to afford 812 mg (77%) of a fluffy white solid. ¹H NMR (400 MHZ, CDCl₃) δ 6.53 (s, 1H), 3.89 (s, 2H), 3.39-3.26 (m, 2H), 2.69 (t, J=7.3 Hz, 2H), 1.88-1.76 (m, 2H), 1.69-1.57 (m, 2H), 1.54-1.42 (m, 2H). SC_ACID: m/z 420.2 [M+H]⁺

### Step 3:

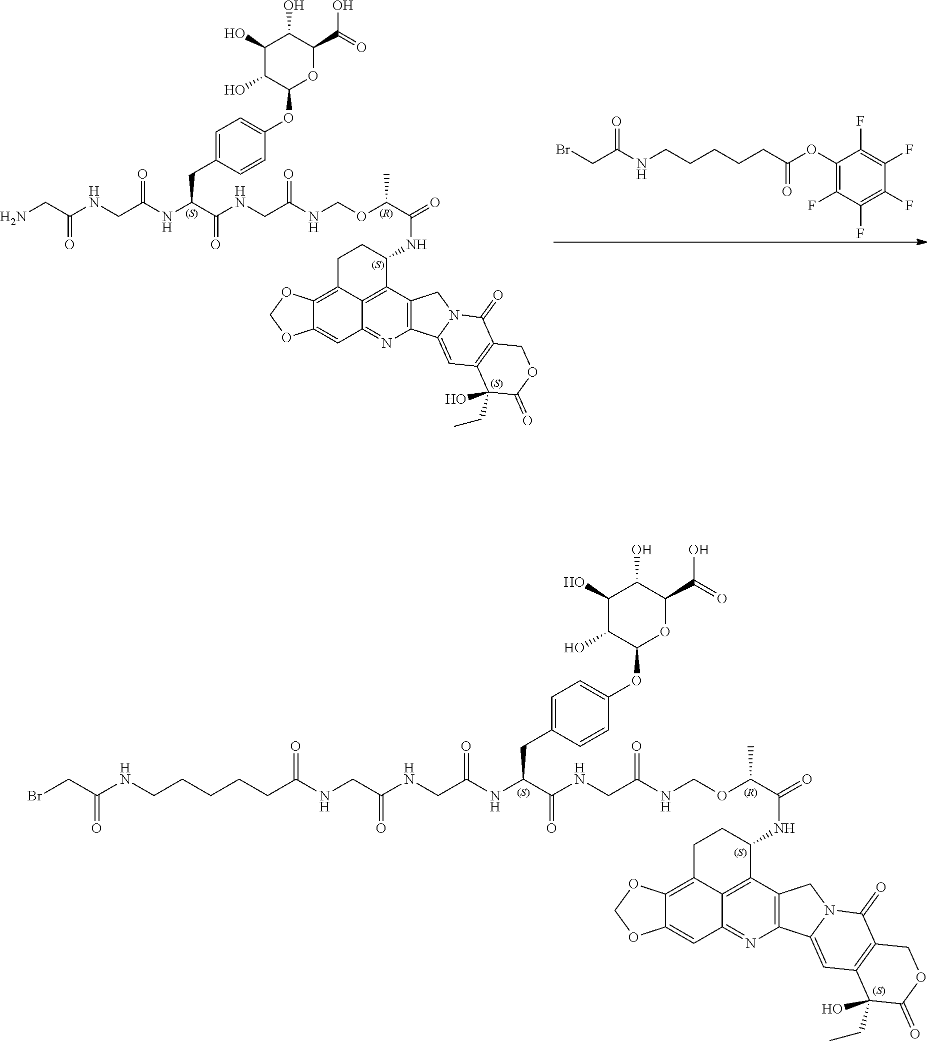

(2S,3S,4S,5R,6S)-6-(4-((S)-2-(2-(2-aminoacetamido)acetamido)-3-((2-(((((R)-1-(((1S, 10S)-10-ethyl-10-hydroxy-11,14-dioxo-2,3,10,11,14,16-hexahydro-1H,13H-benzo[de][1,3]dioxolo[4,5-g]pyrano [3′,4′:6,7]indolizino[1,2-b]quinolin-1-yl)amino)-1-oxopropan-2-yl)oxy)methyl)amino)-2-oxoethyl) amino)-3-oxopropyl)phenoxy)-3,4,5-trihydroxytetrahydro-2H-pyran-2-carboxylic acid (50 mg, 1 eq, 47 μmol) was dissolved in 3.5 mL DMF. perfluorophenyl 6-(2-bromoacetamido)hexanoate (39 mg, 2 eq, 94 μmol) and DIPEA (18 mg, 25 μL, 3 eq, 0.14 mmol) were added. The reaction mixture was stirred at room temperature for 1 hour. The reaction mixture was acidified with acetic acid (10 mg, 10 μL, 3.7 eq, 0.17 mmol) and then purified by acidic preparative MPLC (Luna5-40). Product fractions were combined and lyophilized which yielded 45 mg of a yellow solid. LCMS purity was not sufficient. The product was purified again by acidic preparative MPLC (Luna5-40) which afforded the 19 mg of the desired product as a yellow solid. m/z 1292.2, 1294.2 [M+H]⁺, Br isotope pattern.

### Synthesis of Linker Payload 5 (LP5)

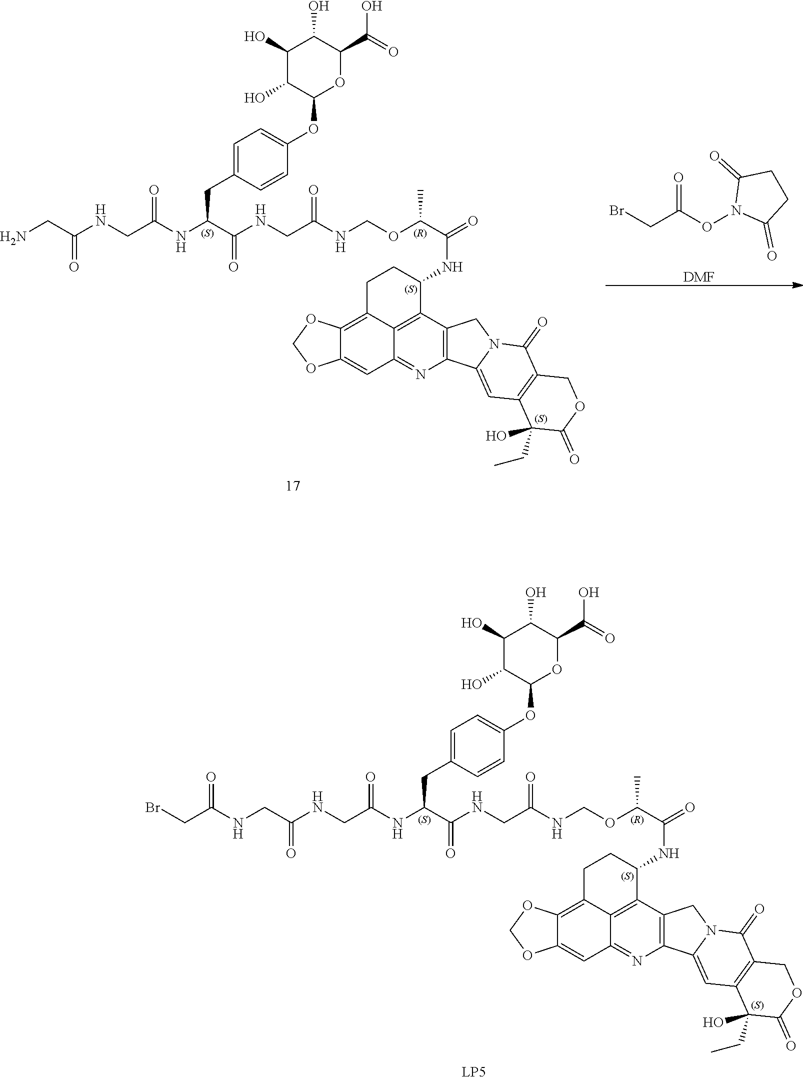

To a solution of (2S,3S,4S,5R,6S)-6-(4-((S)-2-(2-(2-aminoacetamido)acetamido)-3-((2-(((((R)-1-(((1S,10S)-10-ethyl-10-hydroxy-11,14-dioxo-2,3,10,11,14,16-hexahydro-1H, 13H-benzo[de][1,3] dioxolo[4,5-g]pyrano[3′,4′:6,7]indolizino[1,2-b]quinolin-1-yl)amino)-1-oxopropan-2-yl)oxy) methyl)amino)-2-oxoethyl)amino)-3-oxopropyl)phenoxy)-3,4,5-trihydroxytetrahydro-2H-pyran-2-carboxylic acid (64.0 mg, 1 eq, 60.4 μmol) in DMF (2.0 mL) was added 2,5-dioxopyrrolidin-1-yl 2-bromoacetate (18.5 mg, 1.3 eq, 78.6 μmol). The resulting yellow solution was stirred at room temperature for 25 minutes. The reaction mixture was taken up in a syringe and purified directly via acidic preparative MPLC (Luna 5-40). Product fractions were lyophilized to afford 35 mg (49%) as a light-yellow solid. 1H NMR (400 MHZ, DMSO-d₆) δ 8.64 (t, J=6.6 Hz, 1H), 8.57-8.47 (m, 2H), 8.29 (t, J=5.9 Hz, 1H), 8.19-8.03 (m, 2H), 7.39 (s, 1H), 7.23 (s, 1H), 7.11 (d, J=8.7 Hz, 2H), 6.97-6.84 (m, 2H), 6.47 (s, 1H), 6.26 (d, J=4.3 Hz, 2H), 5.61-5.48 (m, 1H), 5.48-5.34 (m, 3H), 5.23-5.03 (m, 3H), 4.97 (d, J=7.4 Hz, 1H), 4.74-4.63 (m, 1H), 4.58-4.37 (m, 2H), 4.16-4.05 (m, 1H), 3.92 (s, 2H), 3.88-3.54 (m, 7H), 3.42-3.35 (m, 1H), 3.29-3.19 (m, 2H), 3.15-2.88 (m, 3H), 2.76-2.58 (m, 1H), 2.18-2.07 (m, 2H), 1.92-1.76 (m, 2H), 1.39 (d, J=6.6 Hz, 3H), 0.87 (t, J=7.3 Hz, 3H); m/z 1179.0, 1181.0 [M+H]+, Br isotope pattern

### Synthesis of Linker Payload 6 (LP6)

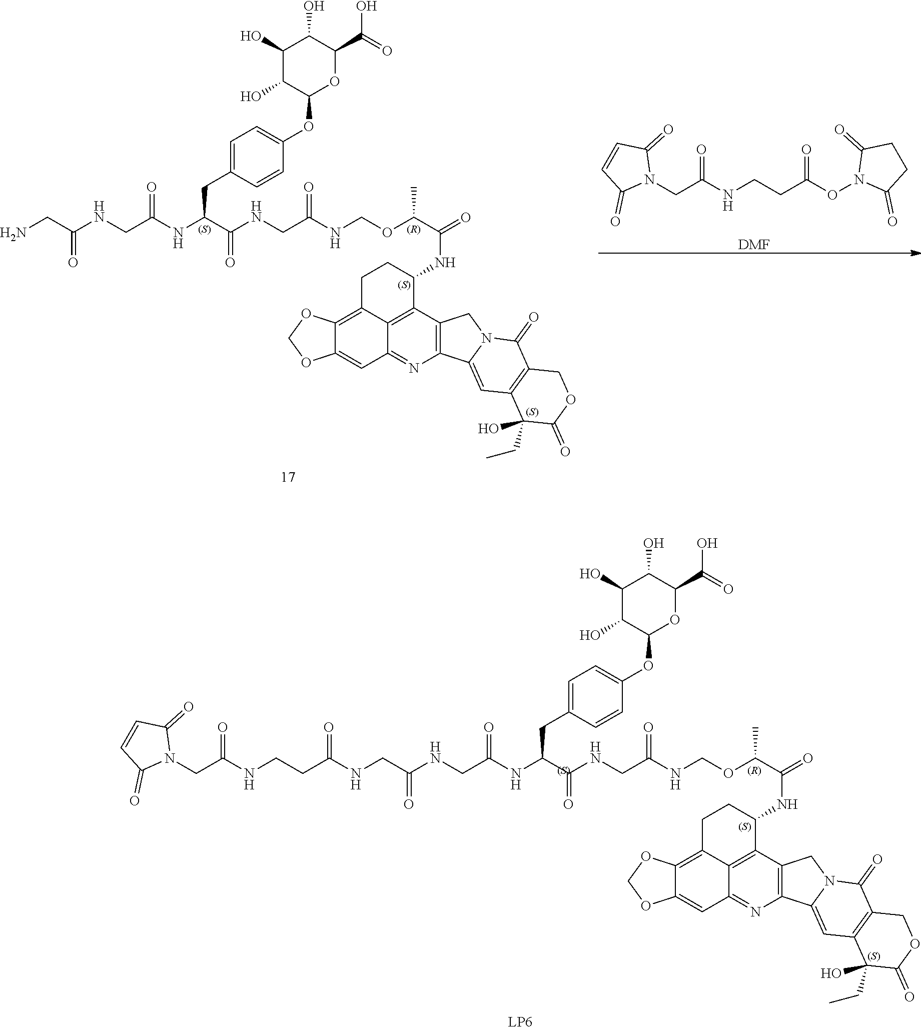

To a solution of (2S,3S,4S,5R,6S)-6-(4-((S)-2-(2-(2-aminoacetamido)acetamido)-3-((2-(((((R)-1-(((1S,10S)-10-ethyl-10-hydroxy-11,14-dioxo-2,3,10,11,14,16-hexahydro-1H,13H-benzo[de][1,3] dioxolo[4,5-g]pyrano[3′,4′:6,7]indolizino[1,2-b]quinolin-1-yl)amino)-1-oxopropan-2-yl)oxy) methyl)amino)-2-oxoethyl)amino)-3-oxopropyl)phenoxy)-3,4,5-trihydroxytetrahydro-2H-pyran-2-carboxylic acid (50 mg, 1 eq, 47 μmol) in DMF (3.0 mL) was added 2,5-dioxopyrrolidin-1-yl 3-(2-(2,5-dioxo-2,5-dihydro-1H-pyrrol-1-yl)acetamido)propanoate (23 mg, 1.5 eq, 71 μmol). After 45 min at room temperature, the reaction mixture was purified by acidic preparative MPLC (Luna5-40). Product fractions were combined and lyophilized to afford 16 mg (27%) of the product as a light-yellow solid. ¹H NMR (400 MHZ, DMSO-d₆) δ 8.63 (t, J=6.6 Hz, 1H), 8.51 (d, J=9.1 Hz, 1H), 8.29 (t, J=5.9 Hz, 1H), 8.26-8.15 (m, 2H), 8.09-8.01 (m, 2H), 7.40 (s, 1H), 7.23 (s, 1H), 7.15-7.04 (m, 4H), 6.92-6.84 (m, 2H), 6.47 (s, 1H), 6.27 (d, J=4.0 Hz, 2H), 5.61-5.52 (m, 1H), 5.43-5.34 (m, 3H), 5.20-5.03 (m, 3H), 4.95 (d, J=7.4 Hz, 1H), 4.73-4.63 (m, 1H), 4.57-4.50 (m, 1H), 4.46-4.37 (m, 1H), 4.15-4.08 (m, 1H), 3.99 (s, 2H), 3.84-3.54 (m, 8H), 3.39-3.34 (m, 1H), 3.29-3.19 (m, 4H), 3.15-2.89 (m, 3H), 2.73-2.63 (m, 1H), 2.30 (t, J=7.1 Hz, 2H), 2.17-2.07 (m, 2H), 1.92-1.78 (m, 2H), 1.39 (d, J=6.8 Hz, 3H), 0.87 (t, J=7.3 Hz, 3H); m/z 1267.4 [M+H]⁺

### Synthesis of Linker Payload 7 (LP7)

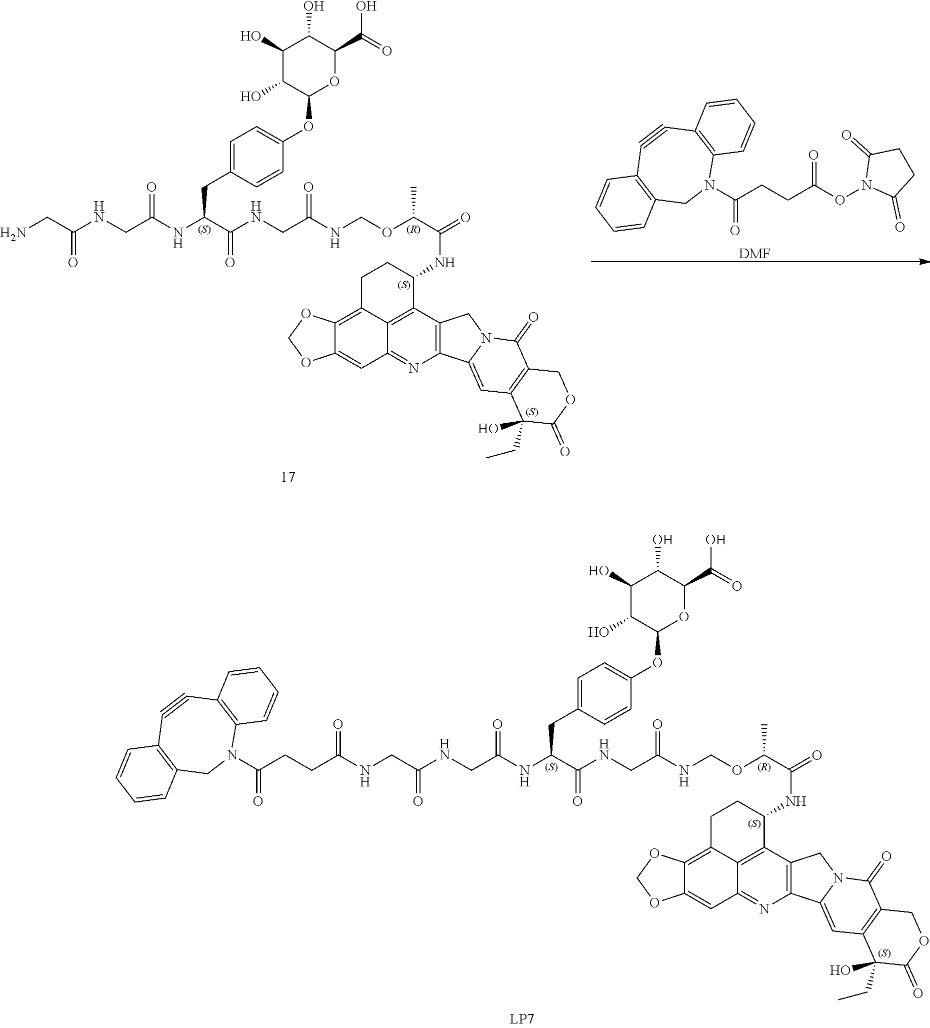

(2S,3S,4S,5R,6S)-6-(4-((S)-2-(2-(2-aminoacetamido)acetamido)-3-((2-(((((R)-1-(((1S,10S)-10-ethyl-10-hydroxy-11,14-dioxo-2,3,10, 11,14,16-hexahydro-1H,13H-benzo[de][1,3]dioxolo[4,5-g] pyrano[3′,4′:6,7]indolizino[1,2-b]quinolin-1-yl)amino)-1-oxopropan-2-yl)oxy)methyl)amino)-2-oxoethyl)amino)-3-oxopropyl)phenoxy)-3,4,5-trihydroxytetrahydro-2H-pyran-2-carboxylic acid (40 mg, 1 eq, 38 μmol) was dissolved in DMF (1.5 mL). DBCO-NHS (15 mg, 1 eq, 38 μmol) was added. The yellow reaction mixture was stirred at room temperature for 1 hour. DBCO-NHS (3.0 mg, 0.2 eq, 7.6 μmol) was added and the mixture stirred at room temperature for 45 minutes. The mixture was purified by acidic preparative MPLC (Luna20-60). Product fractions were combined and lyophilized which afforded 39 mg (77%) as a light-yellow solid. ¹H NMR (400 MHZ, DMSO-d₆) δ 8.69-8.57 (m, 1H), 8.52 (d, J=8.9 Hz, 1H), 8.33-8.21 (m, 1H), 8.19-8.07 (m, 1H), 8.07-7.92 (m, 2H), 7.70-7.53 (m, 2H), 7.49-7.21 (m, 8H), 7.10 (d, J=8.3 Hz, 2H), 6.91-6.84 (m, 2H), 6.48 (s, 1H), 6.29-6.22 (m, 2H), 5.63-5.49 (m, 1H), 5.39 (s, 3H), 5.23-4.89 (m, 5H), 4.72-4.60 (m, 1H), 4.57-4.47 (m, 1H), 4.44-4.34 (m, 1H), 4.17-4.04 (m, 1H), 3.85-3.46 (m, 8H), 3.27-3.18 (m, 2H), 3.14-2.86 (m, 4H), 2.74-2.57 (m, 2H), 2.31-2.21 (m, 1H), 2.18-1.99 (m, 3H), 1.92-1.72 (m, 3H), 1.42-1.35 (m, 3H), 0.86 (t, J=7.3 Hz, 3H); m/z 1346.2 [M+H]⁺

1. A compound of Formula (1): 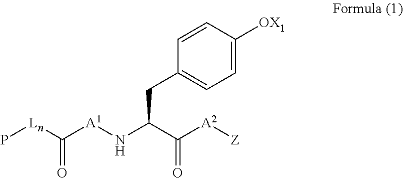
wherein
P is -(maleimide-N)—, -(dibromomaleimide-N)—, -(bromoacetamide-N)—, —(Y—CH₂)—, or -(dibenzocyclooctyne-N—C(═O))—, Y being Br, Cl or I, and the -(dibenzocyclooctyne-N—C(═O))— having the structure of 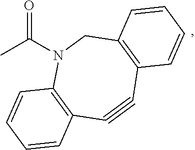 [SMILES: CC(=O)N1Cc2ccccc2C#Cc2ccccc21]
Lₙ is optionally included, and is cycloalkyl, alkyl, or —(OCH₂CH₂)—, —(CH₂—C(═O)—NH—CH₂—CH₂)— if included, and n is an integer of 1 to 6;
A¹ is a peptide residue containing 0-3 amino acids;
A² is a peptide residue containing 0-3 amino acids;
Z is optionally included, and is a self-immolative spacer, —(NHCH₂)—, or p-aminocarbamate if included; and
X₁ is H, monosaccharide, disaccharide, oligosaccharide, polyethylene glycol, sulfate, phosphate, or pyrophosphate.

2. A linker-drug conjugate comprising:
the compound according to claim 1; and
a drug which is conjugated to the compound via Z of Formula (1).

3. The linker-drug conjugate according to claim 2, wherein the drug is conjugated to the compound in the form of —(NH-drug)- or —(O-drug)-.

4. The linker-drug conjugate according to claim 2, wherein the drug is a topoisomerase 1 inhibitor.

5. An antibody-drug conjugate comprising an antibody or an antigen binding fragment thereof, a drug, and a linker,
wherein the linker is the compound according to claim 1,
wherein the antibody or the antigen binding fragment thereof is conjugated to the compound via P of Formula (1), and
wherein the drug is conjugated to the compound via Z of Formula (1).

6. A method of treating cancer, the method comprising administering an effective amount of the antibody-drug conjugate of claim 5 to a subject in need thereof.

7. The method of claim 6, wherein the cancer comprises one or more of breast cancer, liver cancer, skin cancer, ovarian cancer, cervical cancer, prostate cancer, testicular cancer, brain cancer, clear cell renal cell carcinoma, glioma, melanoma, lung cancer, non-small cell lung cancer (NSCLC), small cell lung cancer, pancreatic cancer, gastric cancer, acute myeloid leukemia (AML), Hodgkin's lymphoma, non-Hodgkin's lymphoma (NHL), colorectal cancer, colon cancer, renal cancer, esophageal cancer, leukaemia, hepatocellular carcinoma, bone cancer, bladder cancer, sarcomas, kidney cancer, head and neck cancer, hypopharyngeal squamous cell carcinoma, glioblastoma, neuroblastoma, endometrial cancer, and urothelial cell carcinoma.

8. A compound of Formula (2): 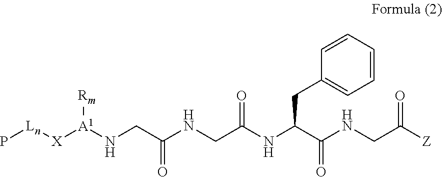
wherein
P is -(maleimide-N)—, -(dibromomaleimide-N)—, -(bromoacetamide-N)—, or -(dibenzocyclooctyne-N—C(═O))—;
Lₙ is optionally included, and is cycloalkyl, alkyl or a direct bond if included, and n is an integer of 1 to 6;
X is —(C═O)—, a direct bond, or a direct bond to a side chain of A¹;
A¹ is an amino acid;
Rₘ is a hydrophilic appendage attached to A¹ and m represents an integer of 1 to 10; and
Z is optionally included, and is a self-immolative spacer, —(NHCH₂)—, or p-aminocarbamate if included.

9. The compound according to claim 8, wherein the amino acid is aspartic acid, glycine, glutamic acid or lysine.

10. A linker-drug conjugate comprising:
the compound according to claim 8; and
a drug which is conjugated to the compound via Z of Formula (2).

11. The linker-drug conjugate according to claim 10, wherein the drug is conjugated to the compound in the form of —(NH-drug)- or —(O-drug)-.

12. The linker-drug conjugate according to claim 10, wherein the drug is a topoisomerase 1 inhibitor.

13. An antibody-drug conjugate comprising an antibody or an antigen binding fragment thereof, a drug, and a linker,
wherein the linker is the compound according to claim 8,
wherein the antibody or the antigen binding fragment thereof is conjugated to the compound via P of Formula (2), and
wherein the drug is conjugated to the compound via Z of Formula (2).

14. A method of treating cancer, the method comprising administering an effective amount of the antibody-drug conjugate of claim 13 to a subject in need thereof.

15. The method of claim 14, wherein the cancer comprises one or more of breast cancer, liver cancer, skin cancer, ovarian cancer, cervical cancer, prostate cancer, testicular cancer, brain cancer, clear cell renal cell carcinoma, glioma, melanoma, lung cancer, non-small cell lung cancer (NSCLC), small cell lung cancer, pancreatic cancer, gastric cancer, acute myeloid leukemia (AML), Hodgkin's lymphoma, non-Hodgkin's lymphoma (NHL), colorectal cancer, colon cancer, renal cancer, esophageal cancer, leukaemia, hepatocellular carcinoma, bone cancer, bladder cancer, sarcomas, kidney cancer, head and neck cancer, hypopharyngeal squamous cell carcinoma, glioblastoma, neuroblastoma, endometrial cancer, and urothelial cell carcinoma.

16. A compound of Formula (3): 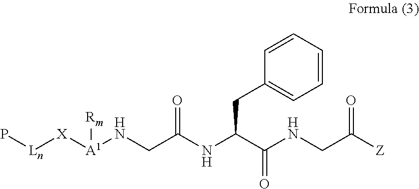
wherein
P is -(maleimide-N)—, -(dibromomaleimide-N)—, -(bromoacetamide-N)—, or -(dibenzocyclooctyne-N—C(═O))—;
Lₙ is optionally included, and is cycloalkyl, alkyl or a direct bond if included, and n is an integer of 1 to 6;
X is —(C═O)—, or a direct bond;
A¹ is an amino acid;
Rₘ is optionally included, and is a hydrophilic appendage attached to A¹ if included, and m represents an integer of 1 to 10; and
Z is optionally included, and is a self-immolative spacer, —(NHCH₂)—, or p-aminocarbamate if included.

17. The compound according to claim 16, wherein the amino acid is aspartic acid, glycine, glutamic acid or lysine.

18. A linker-drug conjugate comprising:
the compound according to claim 16; and
a drug which is conjugated to the compound via Z of Formula (2).

19. The linker-drug conjugate according to claim 18, wherein the drug is conjugated to the compound in the form of —(NH-drug)- or —(O-drug)-.

20. The linker-drug conjugate according to claim 18, wherein the drug is a topoisomerase 1 inhibitor.

21. An antibody-drug conjugate comprising an antibody or an antigen binding fragment thereof, a drug, and a linker,
wherein the linker is the compound according to claim 16,
wherein the antibody or the antigen binding fragment thereof is conjugated to the compound via P of Formula (3), and
wherein the drug is conjugated to the compound via Z of Formula (3).

22. A method of treating cancer, the method comprising administering an effective amount of the antibody-drug conjugate of claim 21 to a subject in need thereof.

23. The method of claim 22, wherein the cancer comprises one or more of breast cancer, liver cancer, skin cancer, ovarian cancer, cervical cancer, prostate cancer, testicular cancer, brain cancer, clear cell renal cell carcinoma, glioma, melanoma, lung cancer, non-small cell lung cancer (NSCLC), small cell lung cancer, pancreatic cancer, gastric cancer, acute myeloid leukemia (AML), Hodgkin's lymphoma, non-Hodgkin's lymphoma (NHL), colorectal cancer, colon cancer, renal cancer, esophageal cancer, leukaemia, hepatocellular carcinoma, bone cancer, bladder cancer, sarcomas, kidney cancer, head and neck cancer, hypopharyngeal squamous cell carcinoma, glioblastoma, neuroblastoma, endometrial cancer, and urothelial cell carcinoma.
